(19) United States
(12) Patent Application Publication
(10) Pub. No.: US 2021/0376241 A1
(43) Pub. Date: Dec. 2, 2021

(54) AMINE-SUBSTITUTED NAPHTHALENE DERIVATIVES AND ORGANIC LIGHT EMITTING DIODES INCLUDING THE SAME

(71) Applicant: SFC CO., LTD., Cheongju-si (KR)

(72) Inventors: Se-jin LEE, Daejeon (KR); Bong-Hyang LEE, Busan (KR); Taejung YU, Yongin-si (KR); Yeongtae CHOI, Yongin-si (KR); Dajung LEE, Bucheon-si (KR)

(73) Assignee: SFC CO., LTD., Cheongju-si (KR)

(21) Appl. No.: 17/399,259

(22) Filed: Aug. 11, 2021

Related U.S. Application Data
(63) Continuation-in-part of application No. 16/184,491, filed on Nov. 8, 2018.

(30) Foreign Application Priority Data
Nov. 8, 2017 (KR) 10-2017-0148234

(51) Int. Cl. H01L 51/00; C07C 211/58; C09K 11/06; C07C 211/54; C07D 333/76; C07C 211/61; C07D 213/72

(52) U.S. Cl. CPC H01L 51/006; C07C 211/58; C09K 11/06; C07C 211/54; H01L 51/5064; H01L 51/0061; C07C 211/61; C07D 213/72; C07D 333/76

(57) ABSTRACT

Disclosed are amine-substituted naphthalene derivatives and organic light emitting diodes including the same. In the organic light emitting diodes, at least one of the amine-substituted naphthalene derivatives is employed in a hole auxiliary layer interposed between a hole transport layer and a light emitting layer to enable efficient hole transport to the light emitting layer, achieving high luminous efficiency and long lifetime.

### CROSS REFERENCE TO RELATED APPLICATION(S)

This application is a continuation-in-part of U.S. patent application Ser. No. 16/184,491 filed on Nov. 8, 2018 and claims the benefit under 35 U.S.C. § 119(a) of Korean Patent Application No. 10-2017-0148234 filed on Nov. 8, 2017 in the Korean Intellectual Property Office, the entire disclosure of which is incorporated herein by reference for all purposes.

### BACKGROUND OF THE INVENTION

### 1. Field of the Invention

The present invention relates to amine-substituted naphthalene derivatives and organic light emitting diodes including the same. More specifically, the present invention relates to organic light emitting diodes in which an amine-substituted naphthalene derivative is employed in a hole auxiliary layer interposed between a hole transport layer and a light emitting layer to enable efficient hole transport to the light emitting layer, achieving high luminous efficiency and long lifetime.

### 2. Description of the Related Art

Organic light emitting diodes are self-luminous devices in which electrons injected from an electron injecting electrode (cathode) combine with holes injected from a hole injecting electrode (anode) in a light emitting layer to form excitons, which emit light while releasing energy.

Such organic light emitting diodes have low driving voltage, high luminance, large viewing angle, and fast response speed and can be applied to full-color light emitting flat panel displays. Due to these advantages, organic light emitting diodes have received attention as next-generation light sources.

The above characteristics of organic light emitting diodes are achieved by structural optimization of organic layers of the diodes and are supported by stable and efficient materials for the organic layers, such as hole injecting materials, hole transport materials, light emitting materials, electron transport materials, electron injecting materials, and electron blocking materials. However, more research still needs to be done to develop structurally optimized structures of organic layers for organic light emitting diodes and stable and efficient materials for organic layers of organic light emitting diodes.

A large difference in highest occupied molecular orbital (HOMO) energy level between a hole transport layer and a light emitting layer makes it difficult to transport holes from the hole transport layer to the light emitting layer, leading to the accumulation of holes at the interface between the hole transport layer and the light emitting layer. In an attempt to solve these problems, the introduction of a hole auxiliary layer has been proposed to facilitate hole transport. Under such circumstances, there is a need to develop materials for hole auxiliary layers.

### SUMMARY OF THE INVENTION

Therefore, the present invention intends to provide a material that is employed in a hole auxiliary layer interposed between a hole transport layer and a light emitting layer to fabricate an organic light emitting diode with high luminous efficiency and long lifetime, and an organic light emitting diode including the same.

One aspect of the present invention provides an amine-substituted naphthalene derivative as an organic light emitting compound, represented by Formula A:

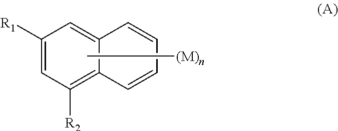

wherein R₁ and R₂ may be identical to or different from each other and are each independently Structure A or B:

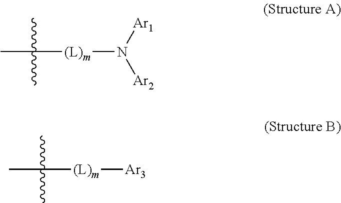

with the proviso that at least one of R₁ and R₂ is Structure A.

The structures and specific substituents of Formula A and Structures A and B are described below.

According to one embodiment of the present invention, either of R₁ and R₂ may be Structure A and the other may be Structure B.

The present invention also provides an organic light emitting diode including a first electrode, a second electrode opposite to the first electrode, and one or more organic layers interposed between the first and second electrodes wherein one of the organic layers includes the naphthalene derivative represented by Formula A and optionally another naphthalene derivative represented by Formula A.

According to one embodiment of the present invention, the naphthalene derivative represented by Formula A may be present in a hole auxiliary layer interposed between the first and second electrodes.

The compound of the present invention is employed in the hole auxiliary layer of the organic light emitting diode to further facilitate hole transport from a hole transport layer to a light emitting layer of the diode, achieving high luminous efficiency and long lifetime of the diode.

### DETAILED DESCRIPTION OF THE INVENTION

The present invention will now be described in more detail.

The present invention is directed to an amine-substituted naphthalene derivative for an organic light emitting diode, represented by Formula A:

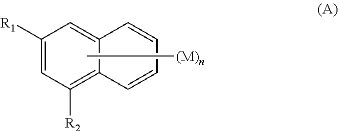

wherein R₁ and R₂ may be identical to or different from each other and are each independently Structure A:

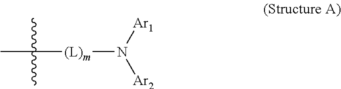

(wherein L represents a linker and is a single bond or is selected from substituted or unsubstituted C₁-C₁₀ alkylene groups, substituted or unsubstituted C₂-C₁₀ alkenylene groups, substituted or unsubstituted C₂-C₁₀ alkynylene groups, substituted or unsubstituted C₃-C₂₀ cycloalkylene groups, substituted or unsubstituted C₂-C₃₀ heterocycloalkylene groups, substituted or unsubstituted C₆-C₃₀ arylene groups, and substituted or unsubstituted C₂-C₃₀ heteroarylene groups, m is an integer from 0 to 4, and Ar₁ and Ar₂ may be identical to or different from each other and are each independently a substituted or unsubstituted C₆-C₅₀ aromatic hydrocarbon ring or a substituted or unsubstituted C₂-C₄₀ aromatic heterocyclic group) or Structure B:

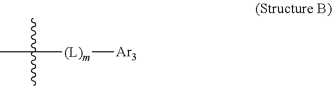

(wherein L and m are as defined in Structure A and Ar₃ is a substituted or unsubstituted C₆-C₅₀ aromatic hydrocarbon ring), with the proviso that at least one of R₁ and R₂ is Structure A,

M is hydrogen, deuterium, a substituted or unsubstituted C₁-C₁₀ alkyl group, a substituted or unsubstituted C₂-C₁₀ alkenyl group, a substituted or unsubstituted C₂-C₁₀ alkynyl group, a substituted or unsubstituted C₃-C₂₀ cycloalkyl group, a substituted or unsubstituted C₅-C₂₀ cycloalkenyl group, a substituted or unsubstituted C₁-C₁₀ alkoxy group, a substituted or unsubstituted C₆-C₃₀ aryloxy group, a substituted or unsubstituted C₁-C₃₀ alkylthioxy group, a substituted or unsubstituted C₅-C₃₀ arylthioxy group, a substituted or unsubstituted C₁-C₃₀ alkylamine group, a substituted or unsubstituted C₅-C₃₀ arylamine group, a substituted or unsubstituted C₆-C₃₀ aryl group, a substituted or unsubstituted C₂-C₃₀ heteroaryl group interrupted by one or more heteroatoms selected from O, N, and S, a cyano group, a nitro group, a halogen group, a substituted or unsubstituted silyl group, a substituted or unsubstituted germanium group, a substituted or unsubstituted boron group, a substituted or unsubstituted aluminum group, a carbonyl group, a phosphoryl group, an amino group, a thiol group, a hydroxyl group, a selenium group, a tellurium group, an amide group or an ester group, and

n is an integer from 0 to 4, provided that when n is equal to or greater than 2, the plurality of M groups may be identical to or different from each other and are each independently optionally combined with an adjacent substituent to form a mono- or polycyclic alicyclic or aromatic ring optionally interrupted by one or more heteroatoms selected from N, S, and O.

Particularly, the amine-substituted naphthalene derivative is employed in a hole auxiliary layer of an organic light emitting diode to further facilitate hole transport from a hole transport layer to a light emitting layer of the diode.

The naphthalene derivative of Formula A structurally has at least one amine substituent (Structure A) at the C-2 (R₁) or C-4 (R₂) position.

According to one embodiment of the present invention, either of R₁ and R₂ may be Structure A and the other may be Structure B.

The expression “substituted or unsubstituted” used in the definition of M and Ar₁ to Ar₃ in Formula A and Structures A and B means that M and Ar₁ to Ar₃ are substituted or unsubstituted with one or more substituents selected from the group consisting of hydrogen, deuterium, a cyano group, a halogen group, a hydroxyl group, a nitro group, alkyl groups, alkoxy groups, alkylamino groups, arylamino groups, heteroarylamino groups, alkylsilyl groups, arylsilyl groups, aryloxy groups, aryl groups, heteroaryl groups, germanium, phosphorus, and boron, or a combination thereof.

In the “substituted or unsubstituted C₁-C₁₀ alkyl group”, “substituted or unsubstituted C₆-C₃₀ aryl group” etc., the number of carbon atoms in the alkyl or aryl group indicates the number of carbon atoms constituting the unsubstituted alkyl or aryl moiety without considering the number of carbon atoms in the substituent(s). For example, a phenyl group substituted with a butyl group at the para-position corresponds to a C₆ aryl group substituted with a C₄ butyl group.

As used herein, the expression “combined with an adjacent substituent to form a ring” means that the corresponding substituent combines with an adjacent substituent to form a substituted or unsubstituted aliphatic or aromatic ring and the term “adjacent substituent” may mean a substituent on an atom directly attached to an atom substituted with the corresponding substituent, a substituent disposed sterically closest to the corresponding substituent or another substituent on an atom substituted with the corresponding substituent. For example, two substituents substituted at the ortho position of a benzene ring or two substituents on the same carbon in an aliphatic ring may be considered “adjacent” to each other.

Specific examples of the aromatic hydrocarbon rings include, but are not limited to, phenyl, naphthyl, and anthracenyl groups. As used herein, the term “aromatic heterocyclic group” refers to an aromatic hydrocarbon ring in which one or more of the aromatic carbon atoms are replaced by heteroatoms such as N, O, P, Si, S, Ge, Se, and Te. The aromatic hydrocarbon rings, the aromatic heterocyclic group, etc. may be monocyclic or polycyclic.

The alkyl groups may be straight or branched. The number of carbon atoms in the alkyl groups is not particularly limited but is preferably from 1 to 20. Specific examples of the alkyl groups include, but are not limited to, methyl, ethyl, propyl, n-propyl, isopropyl, butyl, n-butyl, isobutyl, tert-butyl, sec-butyl, 1-methylbutyl, 1-ethylbutyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethylpropyl, 1,1-dimethylpropyl, isohexyl, 4-methylhexyl, and 5-methylhexyl groups.

The alkenyl group is intended to include straight and branched ones and may be optionally substituted with one or more other substituents. The alkenyl group may be specifically a vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, stilbenyl or styrenyl group but is not limited thereto.

The alkynyl group is intended to include straight and branched ones and may be optionally substituted with one or more other substituents. The alkynyl group may be, for example, ethynyl or 2-propynyl but is not limited thereto.

The cycloalkyl group is intended to include monocyclic and polycyclic ones and may be optionally substituted with one or more other substituents. As used herein, the term “polycyclic” means that the cycloalkyl group may be directly attached or fused to one or more other cyclic groups. The other cyclic groups may be cycloalkyl groups and other examples thereof include heterocycloalkyl, aryl, and heteroaryl groups. The cycloalkyl group may be specifically a cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl or cyclooctyl group but is not limited thereto.

The heterocycloalkyl group is intended to include monocyclic and polycyclic ones interrupted by a heteroatom such as O, S, Se, N or Si and may be optionally substituted with one or more other substituents. As used herein, the term “polycyclic” means that the heterocycloalkyl group may be directly attached or fused to one or more other cyclic groups. The other cyclic groups may be heterocycloalkyl groups and other examples thereof include cycloalkyl, aryl, and heteroaryl groups.

The aryl groups may be monocyclic or polycyclic ones. Examples of the monocyclic aryl groups include, but are not limited to, phenyl, biphenyl, terphenyl, and stilbene groups. Examples of the polycyclic aryl groups include, but are not limited to, naphthyl, anthracenyl, phenanthryl, pyrenyl, perylenyl, tetracenyl, chrysenyl, fluorenyl, naphthacenyl, triphenylenyl, and fluoranthrenyl groups.

The heteroaryl groups refer to heterocyclic groups interrupted by one or more heteroatoms. Examples of the heteroaryl groups include, but are not limited to, thiophene, furan, pyrrole, imidazole, triazole, oxazole, oxadiazole, triazole, pyridyl, bipyridyl, pyrimidyl, triazine, triazole, acridyl, pyridazine, pyrazinyl, quinolinyl, quinazoline, quinoxalinyl, phthalazinyl, pyridopyrimidinyl, pyridopyrazinyl, pyrazinopyrazinyl, isoquinoline, indole, carbazole, benzoxazole, benzimidazole, benzothiazole, benzocarbazole, benzothiophene, dibenzothiophene, benzofuranyl, dibenzofuranyl, phenanthroline, thiazolyl, isoxazolyl, oxadiazolyl, thiadiazolyl, benzothiazolyl, phenothiazinyl groups.

The alkoxy group may be specifically a methoxy, ethoxy, propoxy, isobutyloxy, sec-butyloxy, pentyloxy, iso-amyloxy or hexyloxy group, but is not limited thereto.

The silyl group is intended to include alkyl-substituted silyl groups and aryl-substituted silyl groups. Specific examples of such silyl groups include, but are not limited to, trimethylsilyl, triethylsilyl, triphenylsilyl, trimethoxysilyl, dimethoxyphenylsilyl, diphenylmethylsilyl, diphenylvinylsilyl, methylcyclobutylsilyl, and dimethylfurylsilyl.

The amine groups may be, for example, —NH₂, alkylamine groups, and arylamine groups. The arylamine groups are aryl-substituted amine groups and the alkylamine groups are alkyl-substituted amine groups. Examples of the arylamine groups include substituted or unsubstituted monoarylamine groups, substituted or unsubstituted diarylamine groups, and substituted or unsubstituted triarylamine groups. The aryl groups in the arylamine groups may be monocyclic or polycyclic ones. The arylamine groups may include two or more aryl groups. In this case, the aryl groups may be monocyclic aryl groups or polycyclic aryl groups. Alternatively, the aryl groups may consist of a monocyclic aryl group and a polycyclic aryl group. The aryl groups in the arylamine groups may be selected from those exemplified above.

The aryl groups in the aryloxy group and the arylthioxy group are the same as those described above. Specific examples of the aryloxy groups include, but are not limited to, phenoxy, p-tolyloxy, m-tolyloxy, 3,5-dimethylphenoxy, 2,4,6-trimethylphenoxy, p-tert-butylphenoxy, 3-biphenyloxy, 4-biphenyloxy, 1-naphthyloxy, 2-naphthyloxy, 4-methyl-1-naphthyloxy, 5-methyl-2-naphthyloxy, 1-anthryloxy, 2-anthryloxy, 9-anthryloxy, 1-phenanthryloxy, 3-phenanthryloxy, and 9-phenanthryloxy groups. The arylthioxy group may be, for example, a phenylthioxy, 2-methylphenylthioxy or 4-tert-butylphenylthioxy group but is not limited thereto.

The halogen group may be, for example, fluorine, chlorine, bromine or iodine.

More specifically, the compound represented by Formula A may be selected from Compounds 1 to 120:

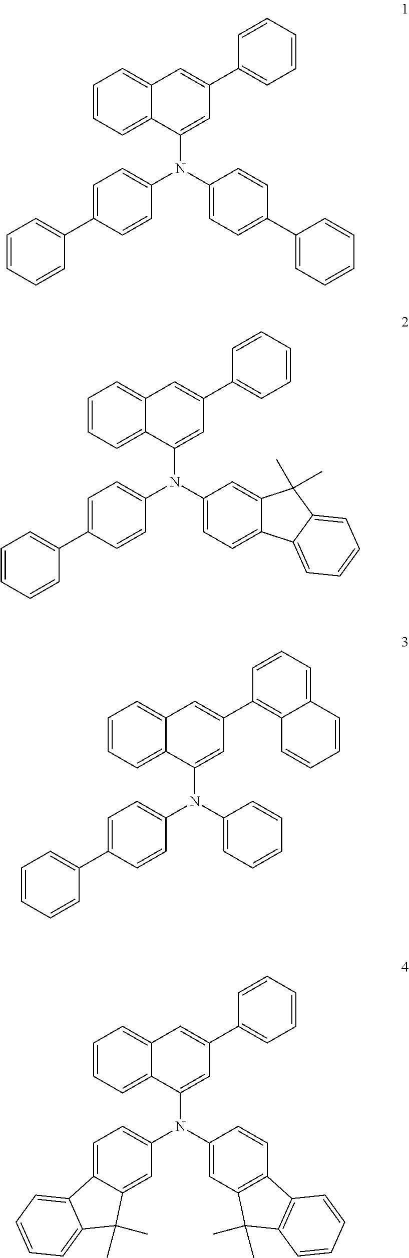 [SMILES: c1ccc(-c2ccc(N(c3ccc(-c4ccccc4)cc3)c3cc(-c4ccccc4)cc4ccccc34)cc2)cc1 ; CC1(C)c2ccccc2-c2ccc(N(c3ccc(-c4ccccc4)cc3)c3cc(-c4ccccc4)cc4ccccc34)cc21 ; c1ccc(-c2ccc(N(c3ccccc3)c3cc(-c4cccc5ccccc45)cc4ccccc34)cc2)cc1 ; CC1(C)c2ccccc2-c2ccc(N(c3ccc4c(c3)C(C)(C)c3ccccc3-4)c3cc(-c4ccccc4)cc4ccccc34)cc21]

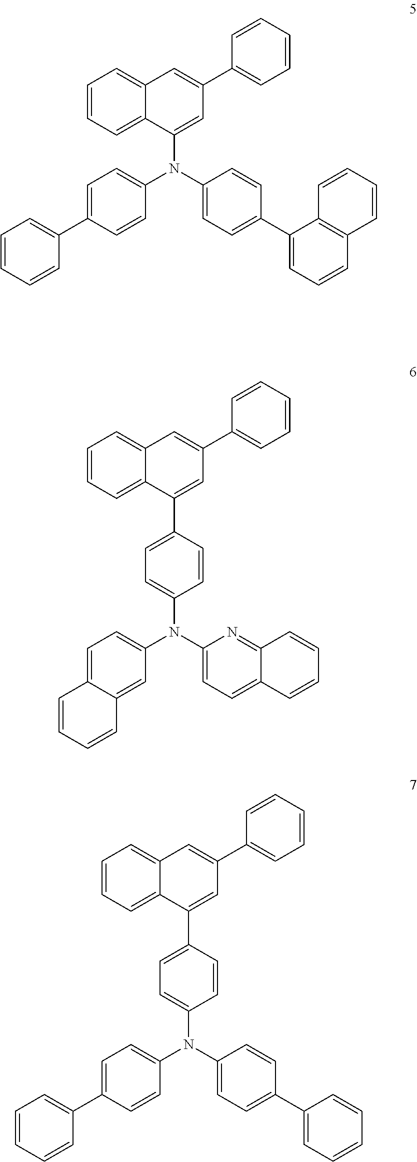 [SMILES: c1ccc(-c2ccc(N(c3ccc(-c4cccc5ccccc45)cc3)c3cc(-c4ccccc4)cc4ccccc34)cc2)cc1 ; c1ccc(-c2cc(-c3ccc(N(c4ccc5ccccc5c4)c4ccc5ccccc5n4)cc3)c3ccccc3c2)cc1 ; c1ccc(-c2ccc(N(c3ccc(-c4ccccc4)cc3)c3ccc(-c4cc(-c5ccccc5)cc5ccccc45)cc3)cc2)cc1]

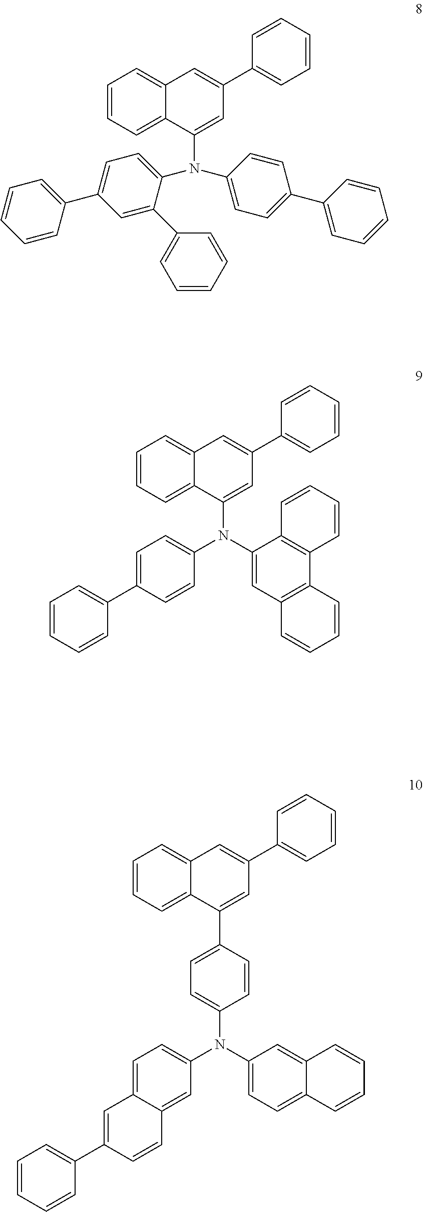 [SMILES: c1ccc(-c2ccc(N(c3ccc(-c4ccccc4)cc3-c3ccccc3)c3cc(-c4ccccc4)cc4ccccc34)cc2)cc1 ; c1ccc(-c2ccc(N(c3cc(-c4ccccc4)cc4ccccc34)c3cc4ccccc4c4ccccc34)cc2)cc1 ; c1ccc(-c2ccc3cc(N(c4ccc(-c5cc(-c6ccccc6)cc6ccccc56)cc4)c4ccc5ccccc5c4)ccc3c2)cc1]

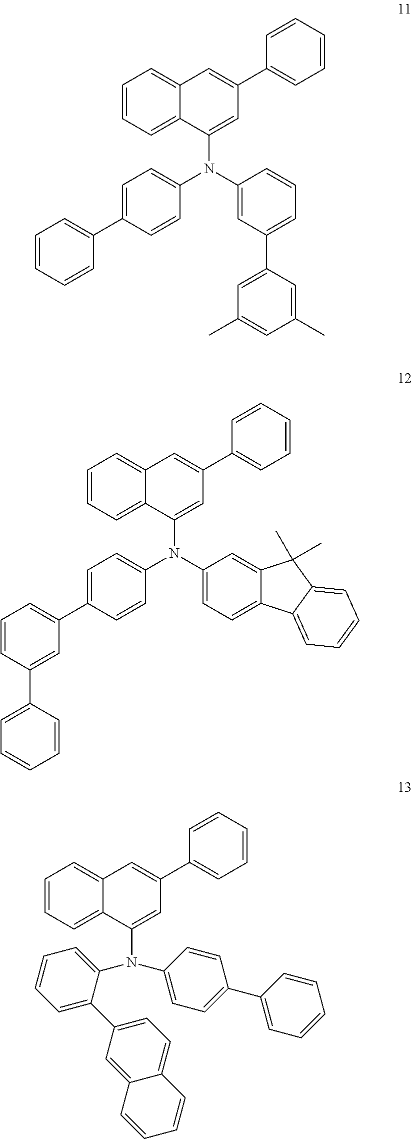 [SMILES: Cc1cc(C)cc(-c2cccc(N(c3ccc(-c4ccccc4)cc3)c3cc(-c4ccccc4)cc4ccccc34)c2)c1 ; CC1(C)c2ccccc2-c2ccc(N(c3ccc(-c4cccc(-c5ccccc5)c4)cc3)c3cc(-c4ccccc4)cc4ccccc34)cc21 ; c1ccc(-c2ccc(N(c3ccccc3-c3ccc4ccccc4c3)c3cc(-c4ccccc4)cc4ccccc34)cc2)cc1]

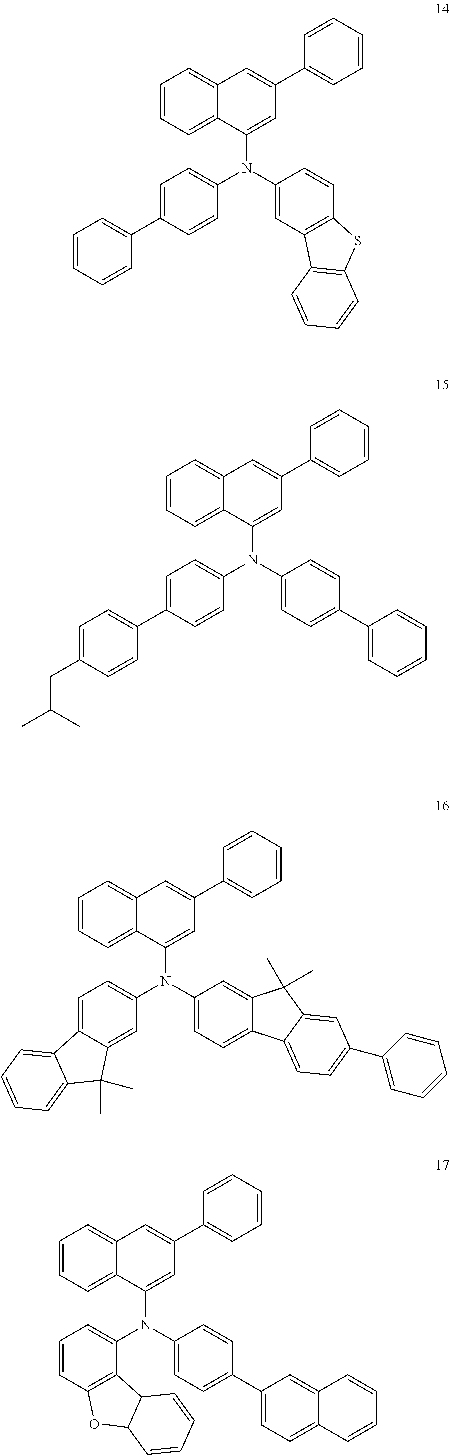 [SMILES: c1ccc(-c2ccc(N(c3ccc4sc5ccccc5c4c3)c3cc(-c4ccccc4)cc4ccccc34)cc2)cc1 ; CC(C)Cc1ccc(-c2ccc(N(c3ccc(-c4ccccc4)cc3)c3cc(-c4ccccc4)cc4ccccc34)cc2)cc1 ; CC1(C)c2ccccc2-c2ccc(N(c3ccc4c(c3)C(C)(C)c3cc(-c5ccccc5)ccc3-4)c3cc(-c4ccccc4)cc4ccccc34)cc21 ; C1=CC2Oc3cccc(N(c4ccc(-c5ccc6ccccc6c5)cc4)c4cc(-c5ccccc5)cc5ccccc45)c3C2C=C1]

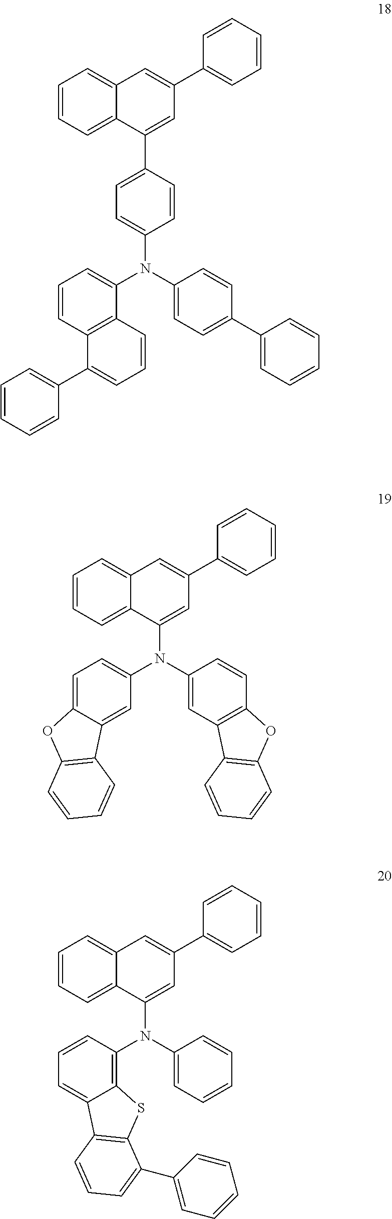 [SMILES: c1ccc(-c2ccc(N(c3ccc(-c4cc(-c5ccccc5)cc5ccccc45)cc3)c3cccc4c(-c5ccccc5)cccc34)cc2)cc1 ; c1ccc(-c2cc(N(c3ccc4oc5ccccc5c4c3)c3ccc4oc5ccccc5c4c3)c3ccccc3c2)cc1 ; c1ccc(-c2cc(N(c3ccccc3)c3cccc4c3sc3c(-c5ccccc5)cccc34)c3ccccc3c2)cc1]

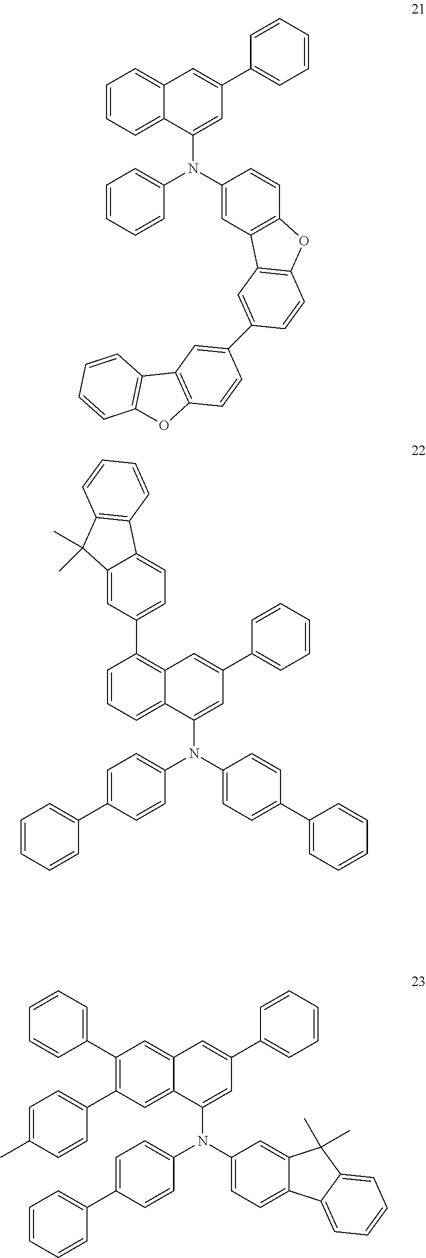

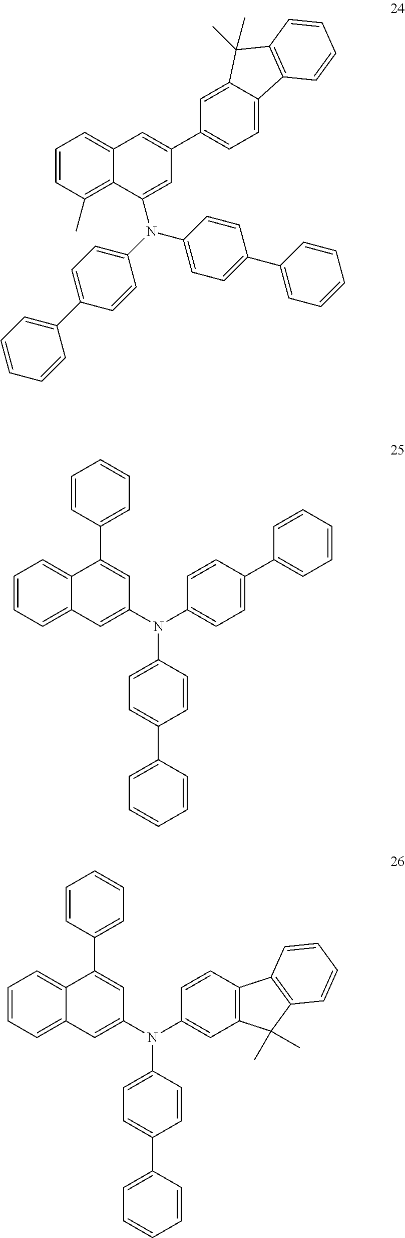 [SMILES: Cc1cccc2cc(-c3ccc4c(c3)C(C)(C)c3ccccc3-4)cc(N(c3ccc(-c4ccccc4)cc3)c3ccc(-c4ccccc4)cc3)c12 ; c1ccc(-c2ccc(N(c3ccc(-c4ccccc4)cc3)c3cc(-c4ccccc4)c4ccccc4c3)cc2)cc1 ; CC1(C)c2ccccc2-c2ccc(N(c3ccc(-c4ccccc4)cc3)c3cc(-c4ccccc4)c4ccccc4c3)cc21]

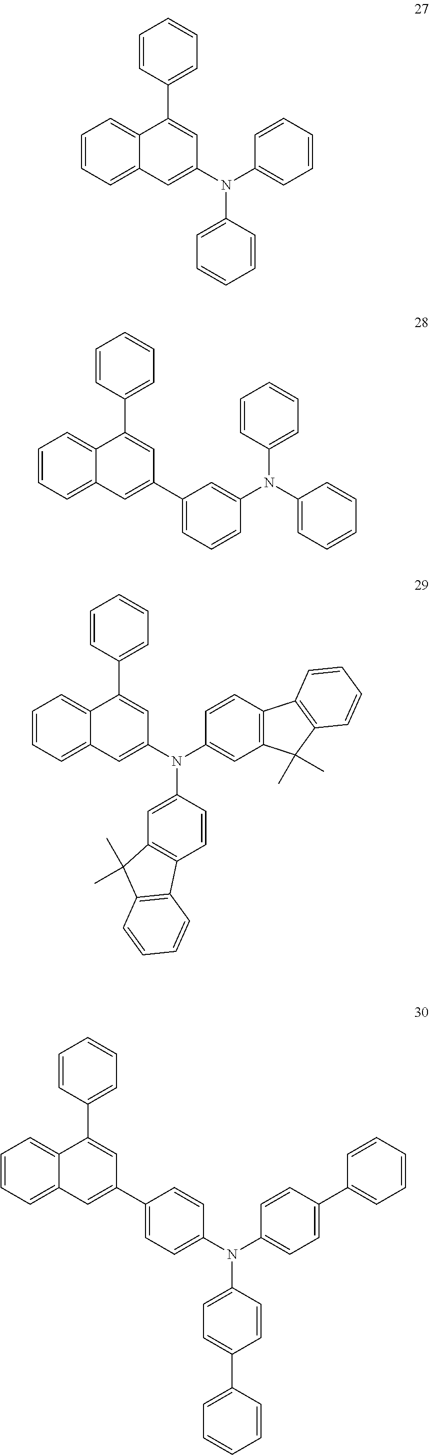 [SMILES: c1ccc(-c2cc(N(c3ccccc3)c3ccccc3)cc3ccccc23)cc1 ; c1ccc(-c2cc(-c3cccc(N(c4ccccc4)c4ccccc4)c3)cc3ccccc23)cc1 ; CC1(C)c2ccccc2-c2ccc(N(c3ccc4c(c3)C(C)(C)c3ccccc3-4)c3cc(-c4ccccc4)c4ccccc4c3)cc21 ; c1ccc(-c2ccc(N(c3ccc(-c4ccccc4)cc3)c3ccc(-c4cc(-c5ccccc5)c5ccccc5c4)cc3)cc2)cc1]

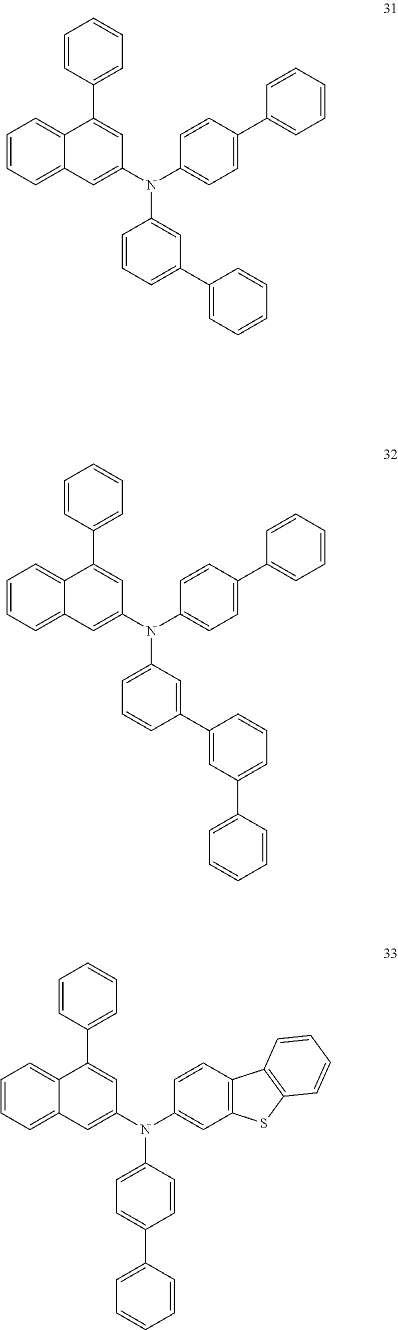 [SMILES: c1ccc(-c2ccc(N(c3cccc(-c4ccccc4)c3)c3cc(-c4ccccc4)c4ccccc4c3)cc2)cc1 ; c1ccc(-c2ccc(N(c3cccc(-c4cccc(-c5ccccc5)c4)c3)c3cc(-c4ccccc4)c4ccccc4c3)cc2)cc1 ; c1ccc(-c2ccc(N(c3cc(-c4ccccc4)c4ccccc4c3)c3ccc4c(c3)sc3ccccc34)cc2)cc1]

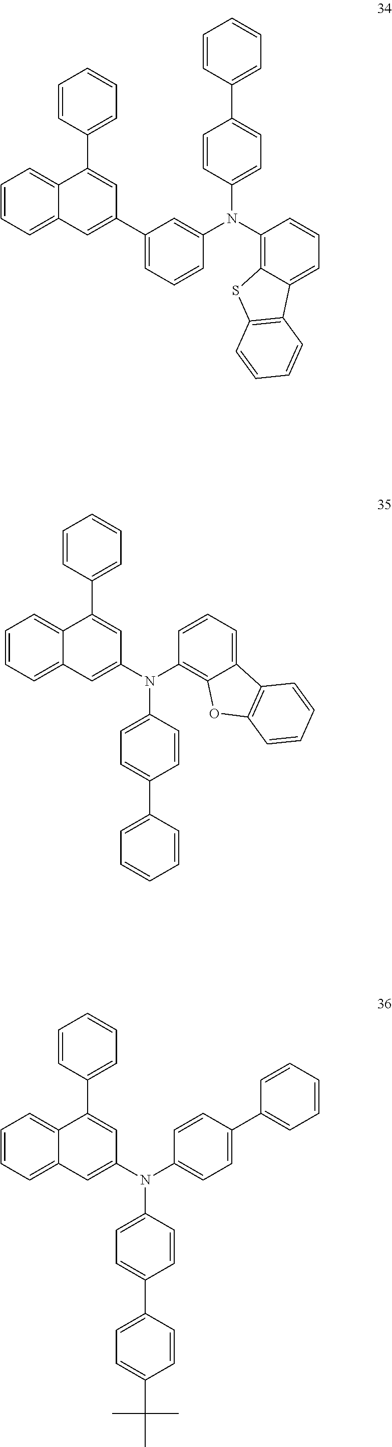 [SMILES: c1ccc(-c2ccc(N(c3cccc(-c4cc(-c5ccccc5)c5ccccc5c4)c3)c3cccc4c3sc3ccccc34)cc2)cc1 ; c1ccc(-c2ccc(N(c3cc(-c4ccccc4)c4ccccc4c3)c3cccc4c3oc3ccccc34)cc2)cc1 ; CC(C)(C)c1ccc(-c2ccc(N(c3ccc(-c4ccccc4)cc3)c3cc(-c4ccccc4)c4ccccc4c3)cc2)cc1]

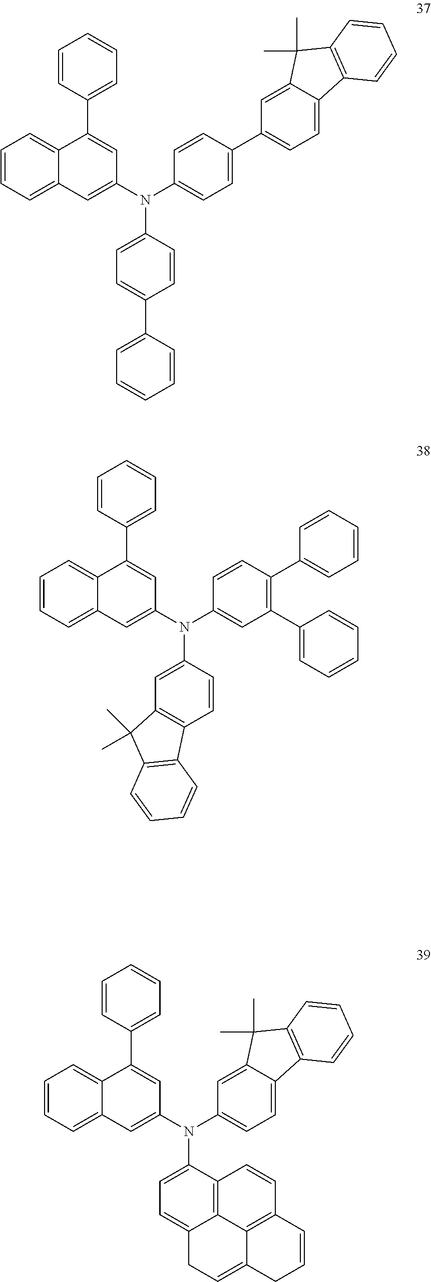 [SMILES: CC1(C)c2ccccc2-c2ccc(-c3ccc(N(c4ccc(-c5ccccc5)cc4)c4cc(-c5ccccc5)c5ccccc5c4)cc3)cc21 ; CC1(C)c2ccccc2-c2ccc(N(c3ccc(-c4ccccc4)c(-c4ccccc4)c3)c3cc(-c4ccccc4)c4ccccc4c3)cc21 ; CC1(C)c2ccccc2-c2ccc(N(c3cc(-c4ccccc4)c4ccccc4c3)c3ccc4c5c6c(ccc35)C=CCC6=CC4)cc21]

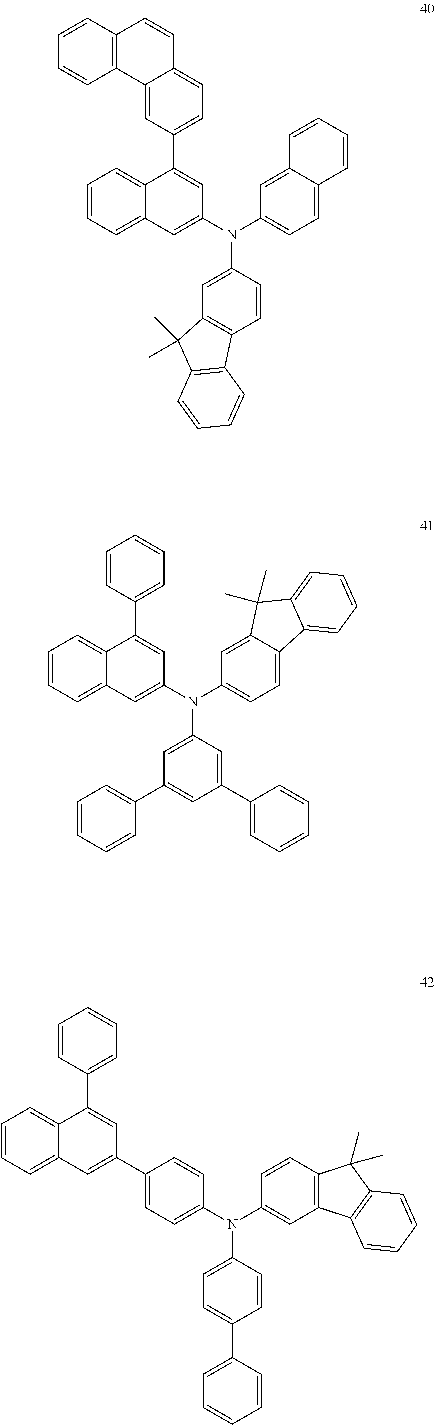 [SMILES: CC1(C)c2ccccc2-c2ccc(N(c3ccc4ccccc4c3)c3cc(-c4ccc5ccc6ccccc6c5c4)c4ccccc4c3)cc21 ; CC1(C)c2ccccc2-c2ccc(N(c3cc(-c4ccccc4)cc(-c4ccccc4)c3)c3cc(-c4ccccc4)c4ccccc4c3)cc21 ; CC1(C)c2ccccc2-c2cc(N(c3ccc(-c4ccccc4)cc3)c3ccc(-c4cc(-c5ccccc5)c5ccccc5c4)cc3)ccc21]

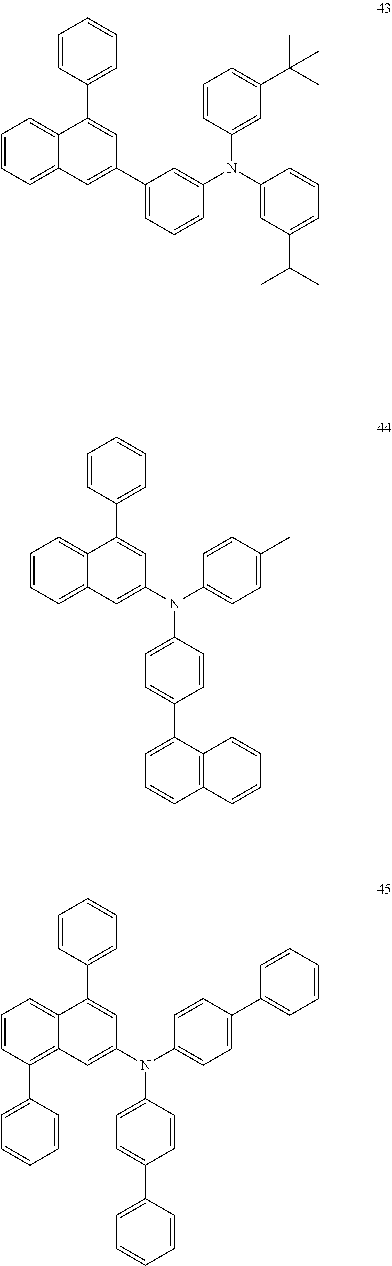 [SMILES: CC(C)c1cccc(N(c2cccc(-c3cc(-c4ccccc4)c4ccccc4c3)c2)c2cccc(C(C)(C)C)c2)c1 ; Cc1ccc(N(c2ccc(-c3cccc4ccccc34)cc2)c2cc(-c3ccccc3)c3ccccc3c2)cc1 ; c1ccc(-c2ccc(N(c3ccc(-c4ccccc4)cc3)c3cc(-c4ccccc4)c4cccc(-c5ccccc5)c4c3)cc2)cc1]

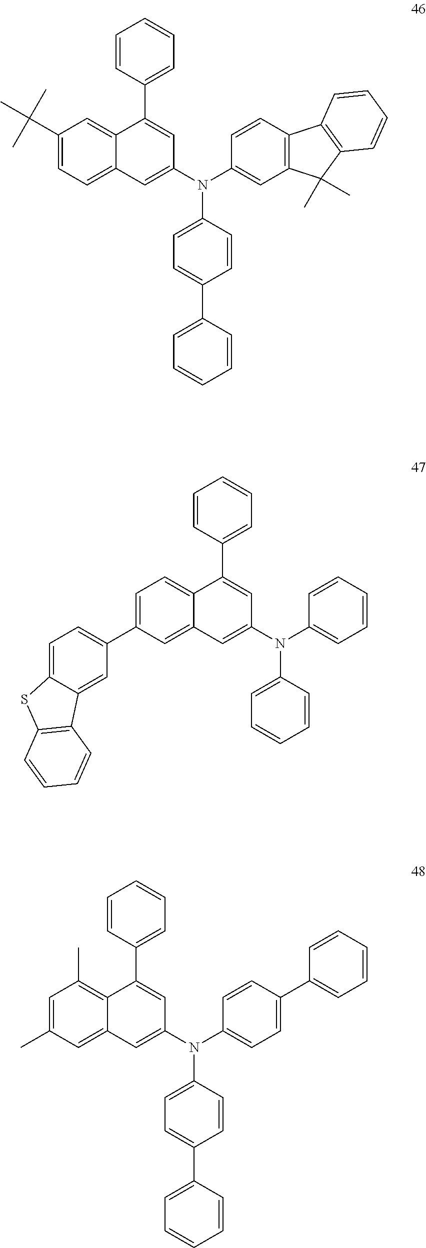 [SMILES: CC(C)(C)c1ccc2cc(N(c3ccc(-c4ccccc4)cc3)c3ccc4c(c3)C(C)(C)c3ccccc3-4)cc(-c3ccccc3)c2c1 ; c1ccc(-c2cc(N(c3ccccc3)c3ccccc3)cc3cc(-c4ccc5sc6ccccc6c5c4)ccc23)cc1 ; Cc1cc(C)c2c(-c3ccccc3)cc(N(c3ccc(-c4ccccc4)cc3)c3ccc(-c4ccccc4)cc3)cc2c1]

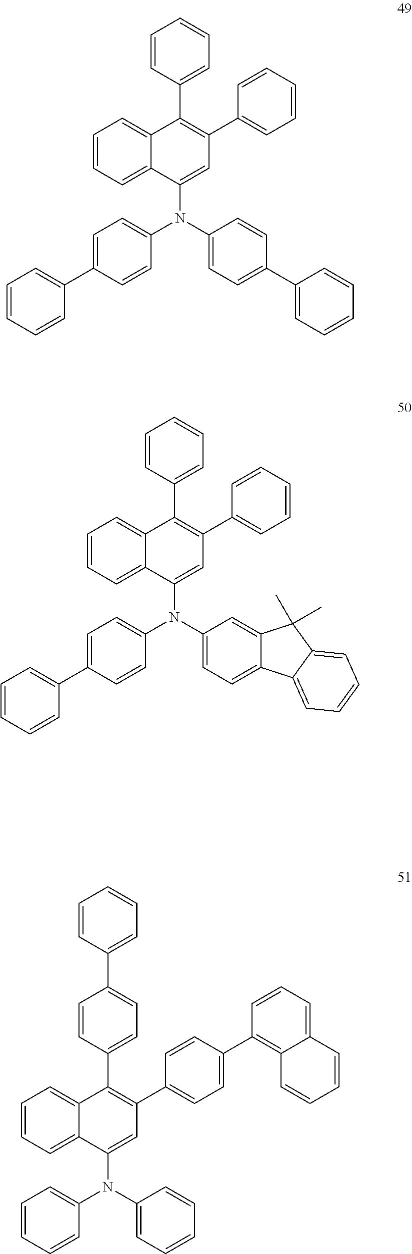 [SMILES: c1ccc(-c2ccc(N(c3ccc(-c4ccccc4)cc3)c3cc(-c4ccccc4)c(-c4ccccc4)c4ccccc34)cc2)cc1 ; CC1(C)c2ccccc2-c2ccc(N(c3ccc(-c4ccccc4)cc3)c3cc(-c4ccccc4)c(-c4ccccc4)c4ccccc34)cc21 ; c1ccc(-c2ccc(-c3c(-c4ccc(-c5cccc6ccccc56)cc4)cc(N(c4ccccc4)c4ccccc4)c4ccccc34)cc2)cc1]

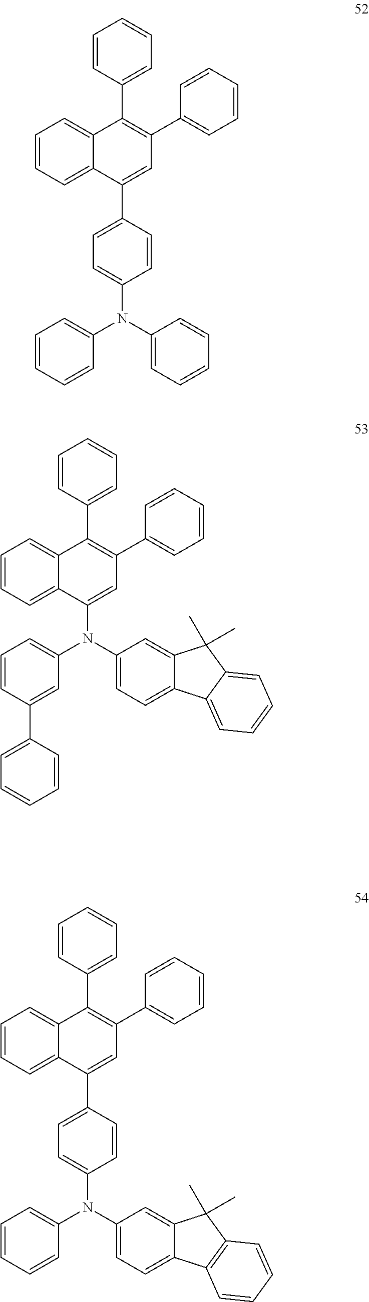 [SMILES: c1ccc(-c2cc(-c3ccc(N(c4ccccc4)c4ccccc4)cc3)c3ccccc3c2-c2ccccc2)cc1 ; CC1(C)c2ccccc2-c2ccc(N(c3cccc(-c4ccccc4)c3)c3cc(-c4ccccc4)c(-c4ccccc4)c4ccccc34)cc21 ; CC1(C)c2ccccc2-c2ccc(N(c3ccccc3)c3ccc(-c4cc(-c5ccccc5)c(-c5ccccc5)c5ccccc45)cc3)cc21]

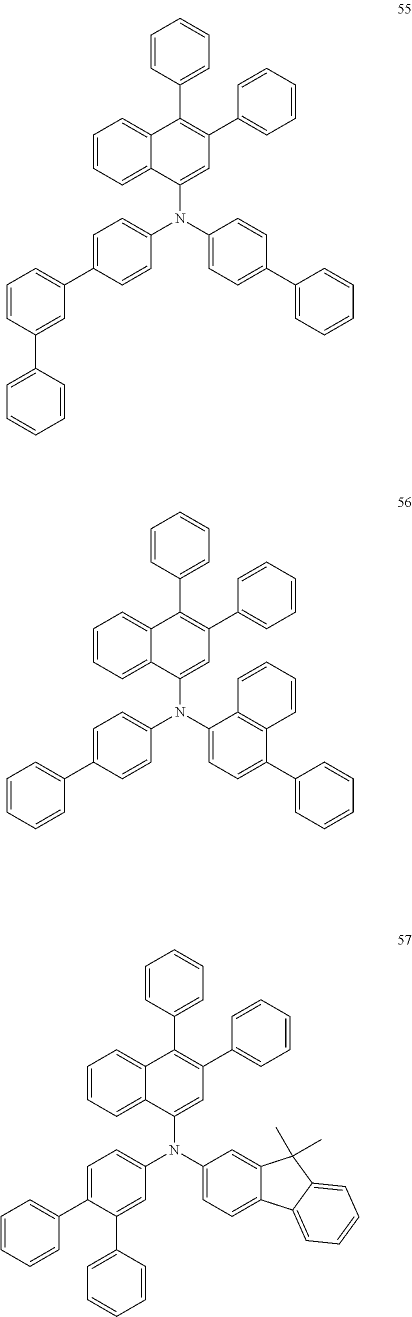 [SMILES: c1ccc(-c2ccc(N(c3ccc(-c4cccc(-c5ccccc5)c4)cc3)c3cc(-c4ccccc4)c(-c4ccccc4)c4ccccc34)cc2)cc1 ; c1ccc(-c2ccc(N(c3ccc(-c4ccccc4)c4ccccc34)c3cc(-c4ccccc4)c(-c4ccccc4)c4ccccc34)cc2)cc1 ; CC1(C)c2ccccc2-c2ccc(N(c3ccc(-c4ccccc4)c(-c4ccccc4)c3)c3cc(-c4ccccc4)c(-c4ccccc4)c4ccccc34)cc21]

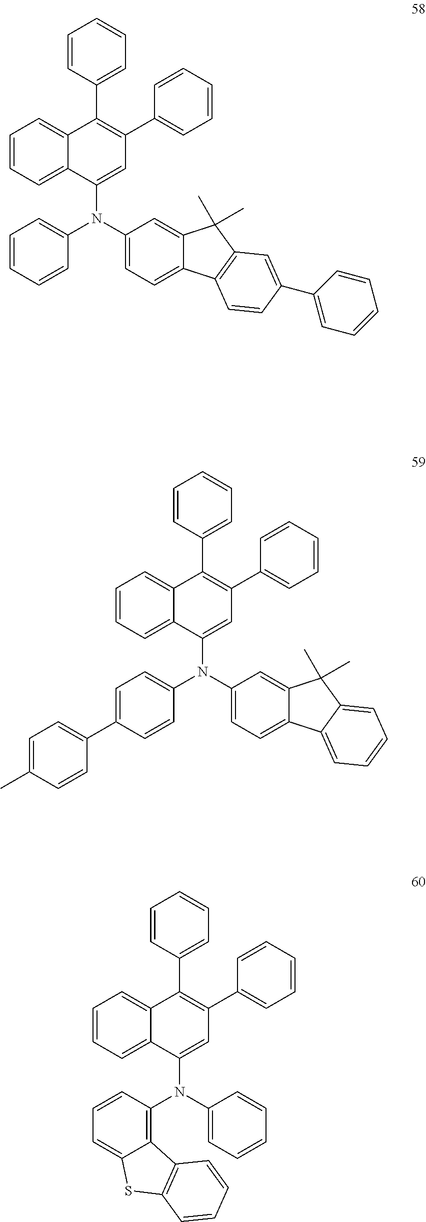 [SMILES: CC1(C)c2cc(-c3ccccc3)ccc2-c2ccc(N(c3ccccc3)c3cc(-c4ccccc4)c(-c4ccccc4)c4ccccc34)cc21 ; Cc1ccc(-c2ccc(N(c3ccc4c(c3)C(C)(C)c3ccccc3-4)c3cc(-c4ccccc4)c(-c4ccccc4)c4ccccc34)cc2)cc1 ; c1ccc(-c2cc(N(c3ccccc3)c3cccc4sc5ccccc5c34)c3ccccc3c2-c2ccccc2)cc1]

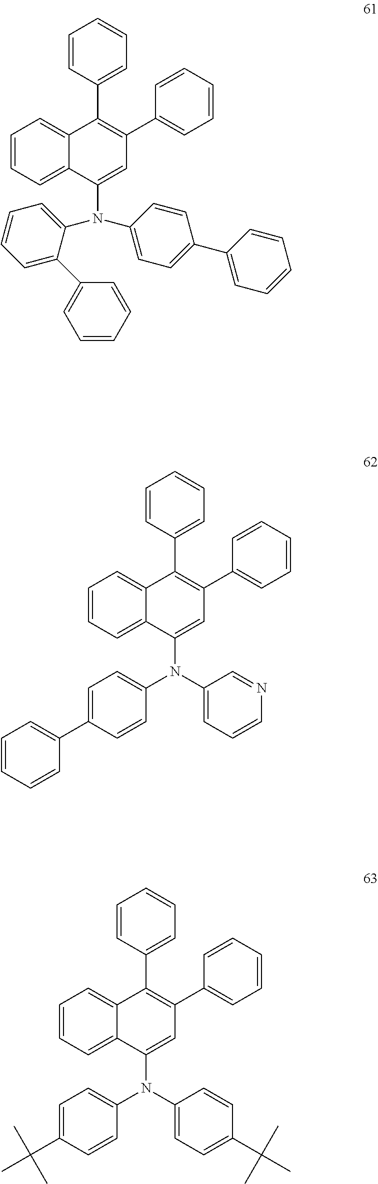 [SMILES: c1ccc(-c2ccc(N(c3ccccc3-c3ccccc3)c3cc(-c4ccccc4)c(-c4ccccc4)c4ccccc34)cc2)cc1 ; c1ccc(-c2ccc(N(c3cccnc3)c3cc(-c4ccccc4)c(-c4ccccc4)c4ccccc34)cc2)cc1 ; CC(C)(C)c1ccc(N(c2ccc(C(C)(C)C)cc2)c2cc(-c3ccccc3)c(-c3ccccc3)c3ccccc23)cc1]

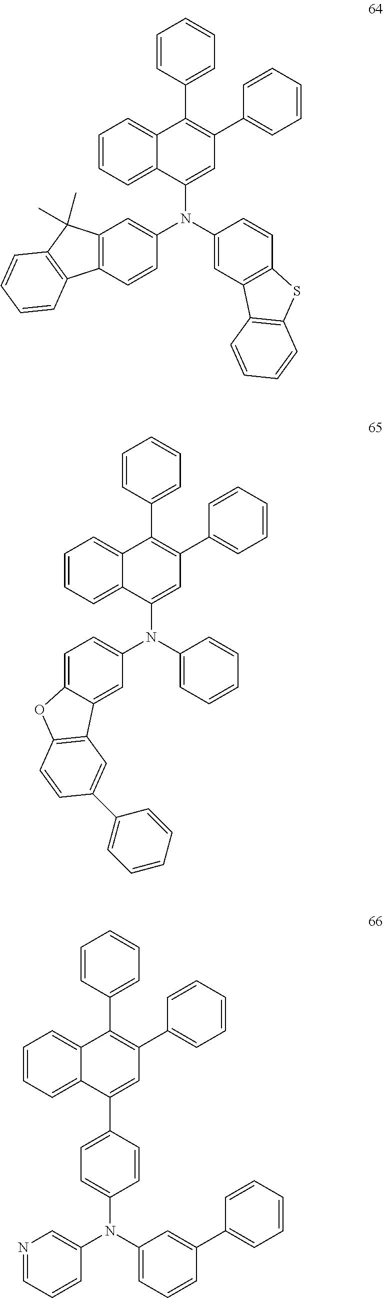 [SMILES: CC1(C)c2ccccc2-c2ccc(N(c3ccc4sc5ccccc5c4c3)c3cc(-c4ccccc4)c(-c4ccccc4)c4ccccc34)cc21 ; c1ccc(-c2ccc3oc4ccc(N(c5ccccc5)c5cc(-c6ccccc6)c(-c6ccccc6)c6ccccc56)cc4c3c2)cc1 ; c1ccc(-c2cccc(N(c3ccc(-c4cc(-c5ccccc5)c(-c5ccccc5)c5ccccc45)cc3)c3cccnc3)c2)cc1]

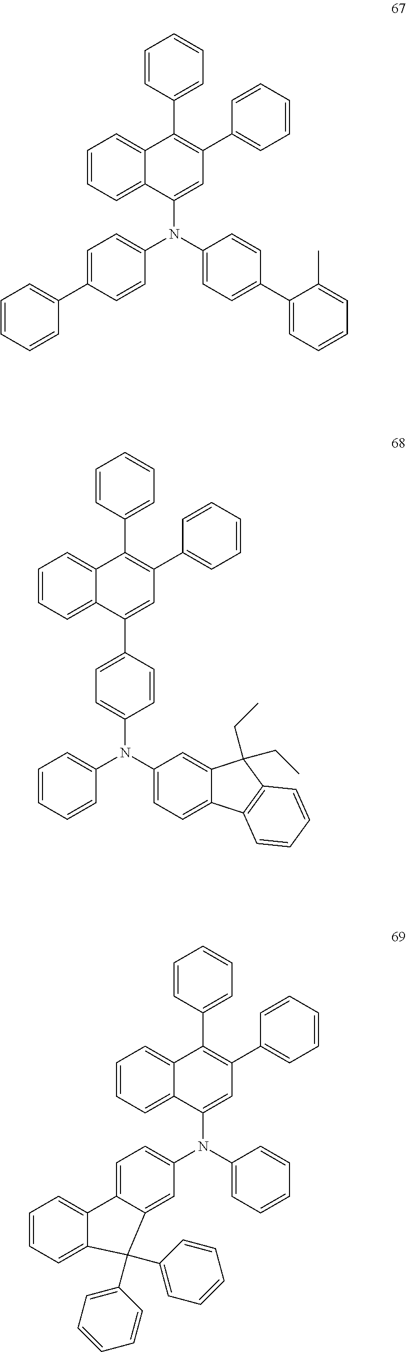 [SMILES: Cc1ccccc1-c1ccc(N(c2ccc(-c3ccccc3)cc2)c2cc(-c3ccccc3)c(-c3ccccc3)c3ccccc23)cc1 ; CCC1(CC)c2ccccc2-c2ccc(N(c3ccccc3)c3ccc(-c4cc(-c5ccccc5)c(-c5ccccc5)c5ccccc45)cc3)cc21 ; c1ccc(-c2cc(N(c3ccccc3)c3ccc4c(c3)C(c3ccccc3)(c3ccccc3)c3ccccc3-4)c3ccccc3c2-c2ccccc2)cc1]

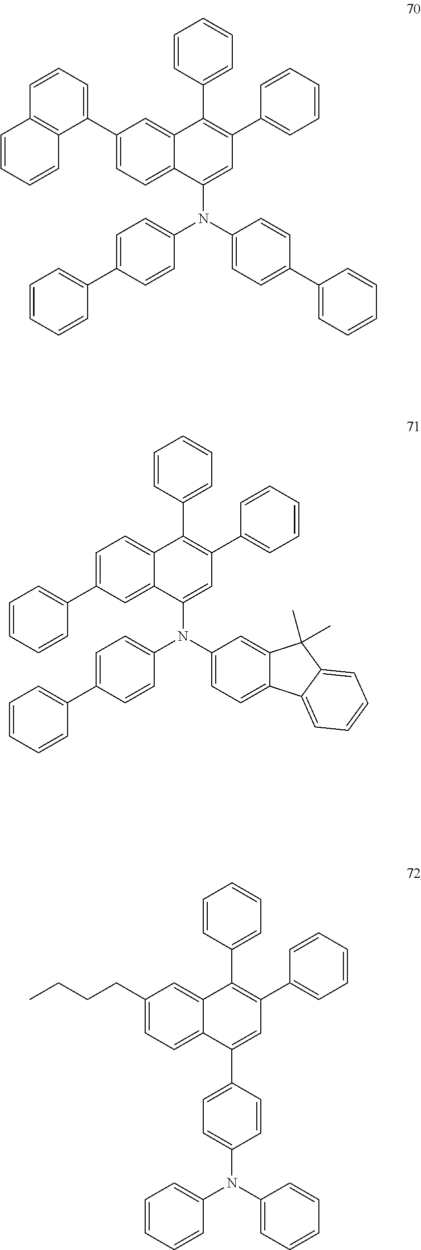 [SMILES: c1ccc(-c2ccc(N(c3ccc(-c4ccccc4)cc3)c3cc(-c4ccccc4)c(-c4ccccc4)c4cc(-c5cccc6ccccc56)ccc34)cc2)cc1 ; CC1(C)c2ccccc2-c2ccc(N(c3ccc(-c4ccccc4)cc3)c3cc(-c4ccccc4)c(-c4ccccc4)c4ccc(-c5ccccc5)cc34)cc21 ; CCCCc1ccc2c(-c3ccc(N(c4ccccc4)c4ccccc4)cc3)cc(-c3ccccc3)c(-c3ccccc3)c2c1]

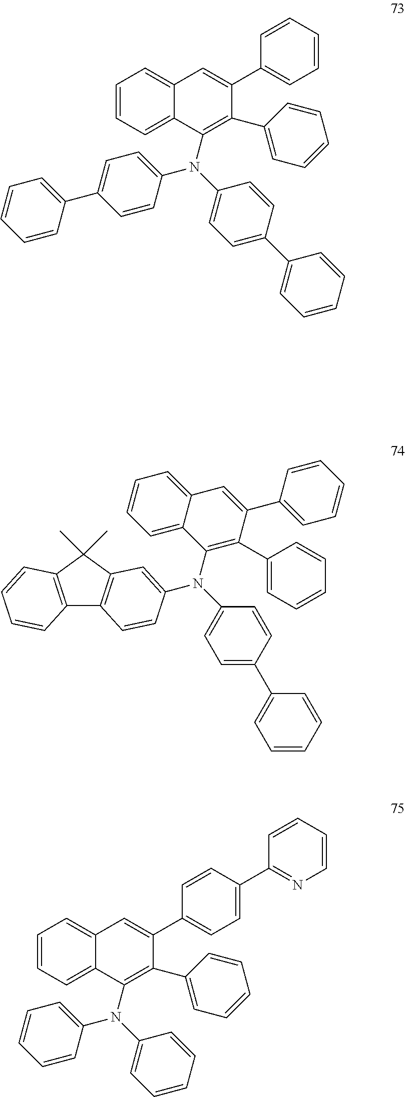 [SMILES: c1ccc(-c2ccc(N(c3ccc(-c4ccccc4)cc3)c3c(-c4ccccc4)c(-c4ccccc4)cc4ccccc34)cc2)cc1 ; CC1(C)c2ccccc2-c2ccc(N(c3ccc(-c4ccccc4)cc3)c3c(-c4ccccc4)c(-c4ccccc4)cc4ccccc34)cc21 ; c1ccc(-c2c(-c3ccc(-c4ccccn4)cc3)cc3ccccc3c2N(c2ccccc2)c2ccccc2)cc1]

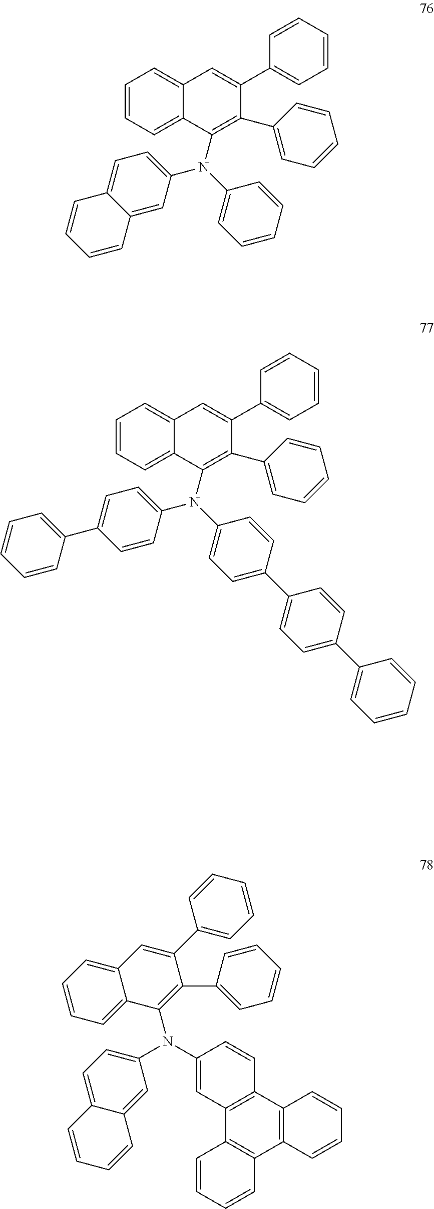 [SMILES: c1ccc(-c2cc3ccccc3c(N(c3ccccc3)c3ccc4ccccc4c3)c2-c2ccccc2)cc1 ; c1ccc(-c2ccc(-c3ccc(N(c4ccc(-c5ccccc5)cc4)c4c(-c5ccccc5)c(-c5ccccc5)cc5ccccc45)cc3)cc2)cc1 ; c1ccc(-c2cc3ccccc3c(N(c3ccc4ccccc4c3)c3ccc4c5ccccc5c5ccccc5c4c3)c2-c2ccccc2)cc1]

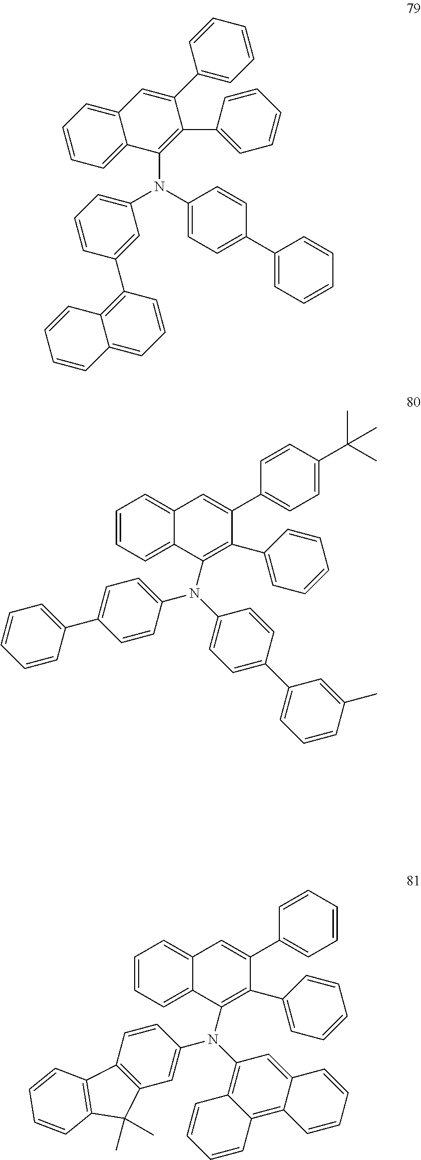 [SMILES: c1ccc(-c2ccc(N(c3cccc(-c4cccc5ccccc45)c3)c3c(-c4ccccc4)c(-c4ccccc4)cc4ccccc34)cc2)cc1 ; Cc1cccc(-c2ccc(N(c3ccc(-c4ccccc4)cc3)c3c(-c4ccccc4)c(-c4ccc(C(C)(C)C)cc4)cc4ccccc34)cc2)c1 ; CC1(C)c2ccccc2-c2ccc(N(c3c(-c4ccccc4)c(-c4ccccc4)cc4ccccc34)c3cc4ccccc4c4ccccc34)cc21]

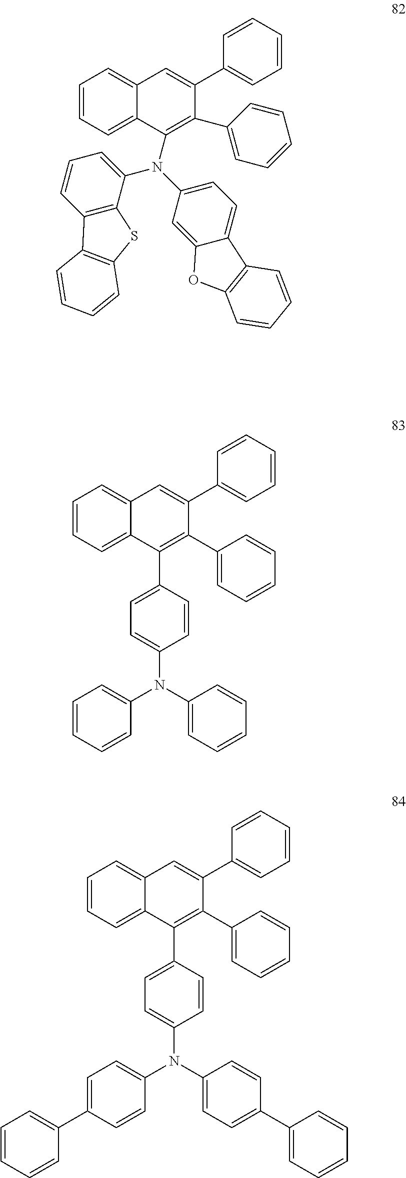 [SMILES: c1ccc(-c2cc3ccccc3c(N(c3ccc4c(c3)oc3ccccc34)c3cccc4c3sc3ccccc34)c2-c2ccccc2)cc1 ; c1ccc(-c2cc3ccccc3c(-c3ccc(N(c4ccccc4)c4ccccc4)cc3)c2-c2ccccc2)cc1 ; c1ccc(-c2ccc(N(c3ccc(-c4ccccc4)cc3)c3ccc(-c4c(-c5ccccc5)c(-c5ccccc5)cc5ccccc45)cc3)cc2)cc1]

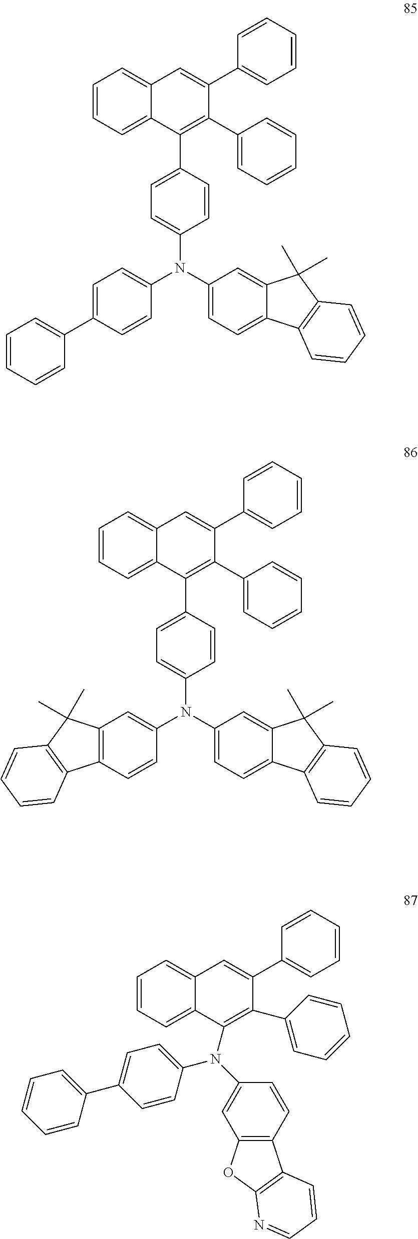 [SMILES: CC1(C)c2ccccc2-c2ccc(N(c3ccc(-c4ccccc4)cc3)c3ccc(-c4c(-c5ccccc5)c(-c5ccccc5)cc5ccccc45)cc3)cc21 ; CC1(C)c2ccccc2-c2ccc(N(c3ccc(-c4c(-c5ccccc5)c(-c5ccccc5)cc5ccccc45)cc3)c3ccc4c(c3)C(C)(C)c3ccccc3-4)cc21 ; c1ccc(-c2ccc(N(c3ccc4c(c3)oc3ncccc34)c3c(-c4ccccc4)c(-c4ccccc4)cc4ccccc34)cc2)cc1]

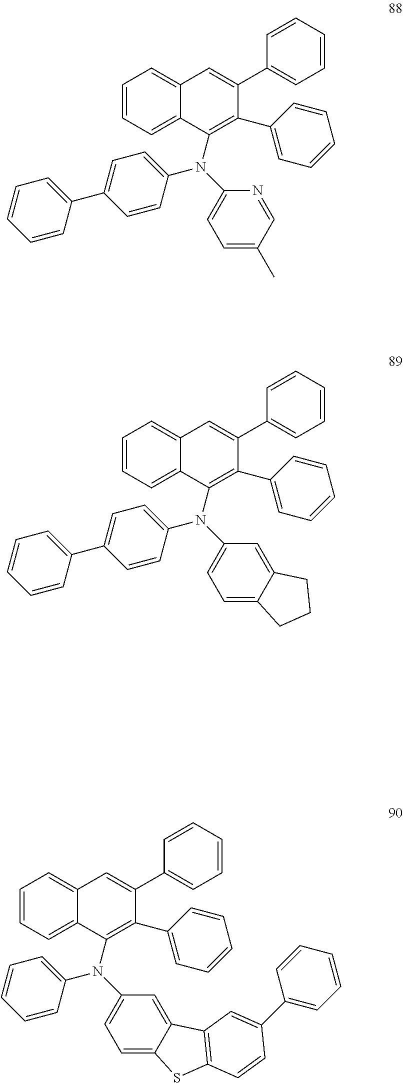 [SMILES: Cc1ccc(N(c2ccc(-c3ccccc3)cc2)c2c(-c3ccccc3)c(-c3ccccc3)cc3ccccc23)nc1 ; c1ccc(-c2ccc(N(c3ccc4c(c3)CCC4)c3c(-c4ccccc4)c(-c4ccccc4)cc4ccccc34)cc2)cc1 ; c1ccc(-c2ccc3sc4ccc(N(c5ccccc5)c5c(-c6ccccc6)c(-c6ccccc6)cc6ccccc56)cc4c3c2)cc1]

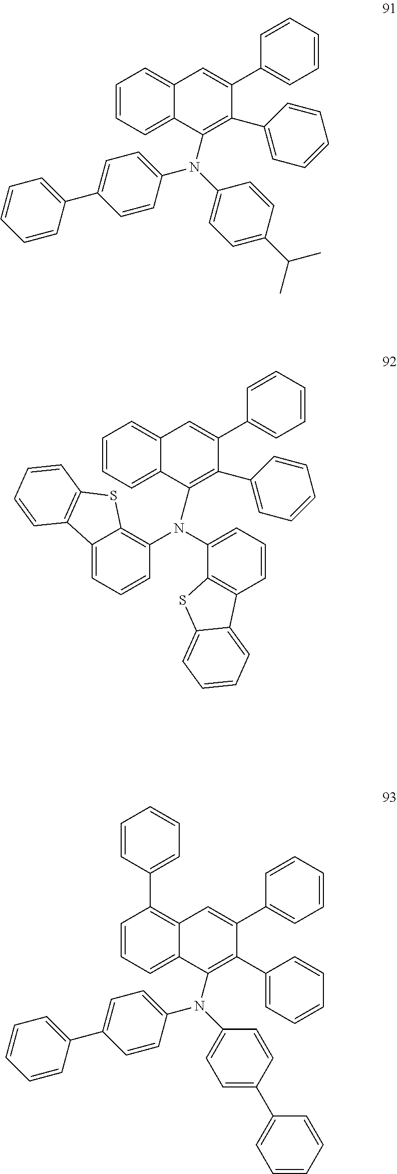 [SMILES: CC(C)c1ccc(N(c2ccc(-c3ccccc3)cc2)c2c(-c3ccccc3)c(-c3ccccc3)cc3ccccc23)cc1 ; c1ccc(-c2cc3ccccc3c(N(c3cccc4c3sc3ccccc34)c3cccc4c3sc3ccccc34)c2-c2ccccc2)cc1 ; c1ccc(-c2ccc(N(c3ccc(-c4ccccc4)cc3)c3c(-c4ccccc4)c(-c4ccccc4)cc4c(-c5ccccc5)cccc34)cc2)cc1]

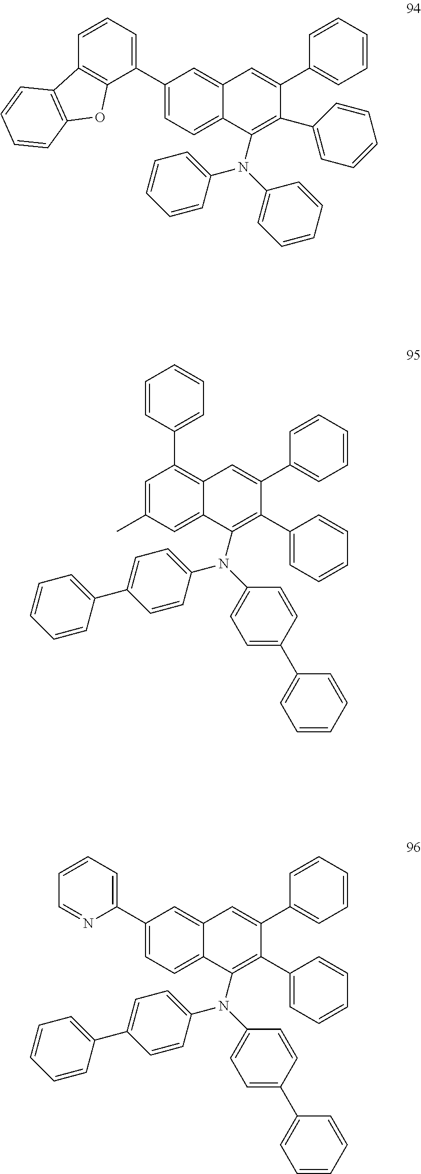 [SMILES: c1ccc(-c2cc3cc(-c4cccc5c4oc4ccccc45)ccc3c(N(c3ccccc3)c3ccccc3)c2-c2ccccc2)cc1 ; Cc1cc(-c2ccccc2)c2cc(-c3ccccc3)c(-c3ccccc3)c(N(c3ccc(-c4ccccc4)cc3)c3ccc(-c4ccccc4)cc3)c2c1 ; c1ccc(-c2ccc(N(c3ccc(-c4ccccc4)cc3)c3c(-c4ccccc4)c(-c4ccccc4)cc4cc(-c5ccccn5)ccc34)cc2)cc1]

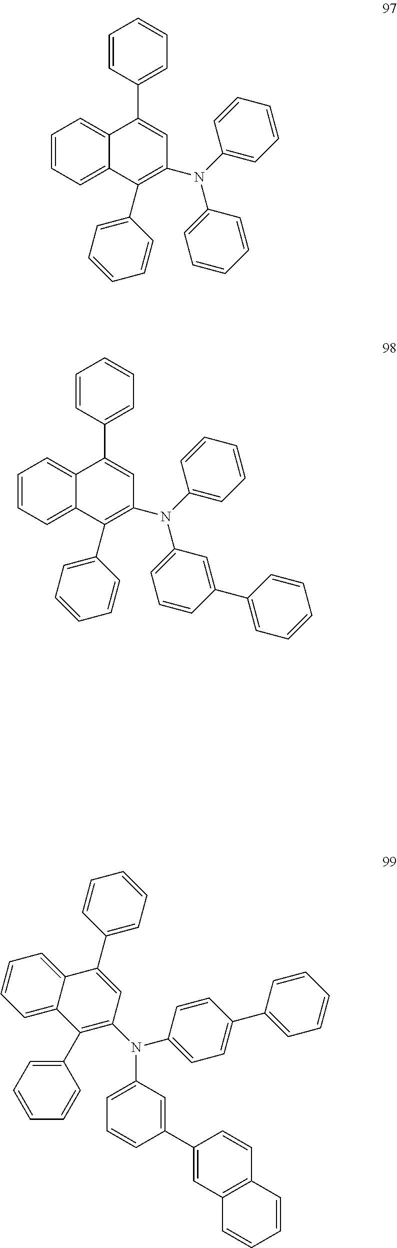 [SMILES: c1ccc(-c2cc(N(c3ccccc3)c3ccccc3)c(-c3ccccc3)c3ccccc23)cc1 ; c1ccc(-c2cccc(N(c3ccccc3)c3cc(-c4ccccc4)c4ccccc4c3-c3ccccc3)c2)cc1 ; c1ccc(-c2ccc(N(c3cccc(-c4ccc5ccccc5c4)c3)c3cc(-c4ccccc4)c4ccccc4c3-c3ccccc3)cc2)cc1]

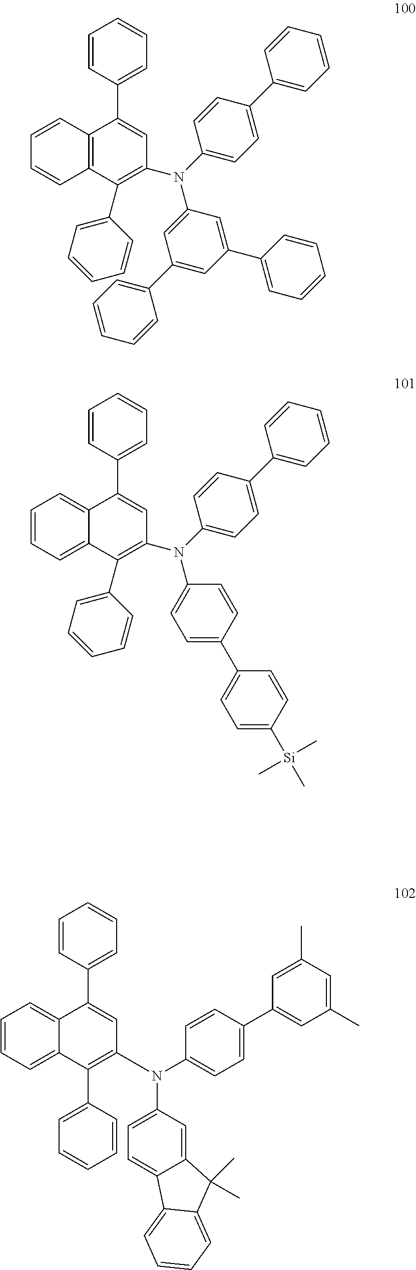 [SMILES: c1ccc(-c2ccc(N(c3cc(-c4ccccc4)cc(-c4ccccc4)c3)c3cc(-c4ccccc4)c4ccccc4c3-c3ccccc3)cc2)cc1 ; C[Si](C)(C)c1ccc(-c2ccc(N(c3ccc(-c4ccccc4)cc3)c3cc(-c4ccccc4)c4ccccc4c3-c3ccccc3)cc2)cc1 ; Cc1cc(C)cc(-c2ccc(N(c3ccc4c(c3)C(C)(C)c3ccccc3-4)c3cc(-c4ccccc4)c4ccccc4c3-c3ccccc3)cc2)c1]

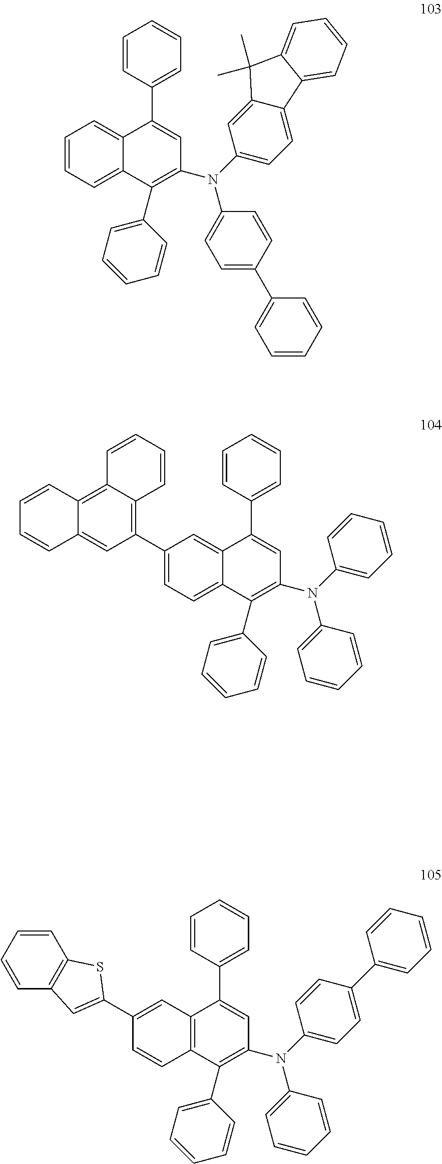 [SMILES: CC1(C)c2ccccc2-c2ccc(N(c3ccc(-c4ccccc4)cc3)c3cc(-c4ccccc4)c4ccccc4c3-c3ccccc3)cc21 ; c1ccc(-c2cc(N(c3ccccc3)c3ccccc3)c(-c3ccccc3)c3ccc(-c4cc5ccccc5c5ccccc45)cc23)cc1 ; c1ccc(-c2ccc(N(c3ccccc3)c3cc(-c4ccccc4)c4cc(-c5cc6ccccc6s5)ccc4c3-c3ccccc3)cc2)cc1]

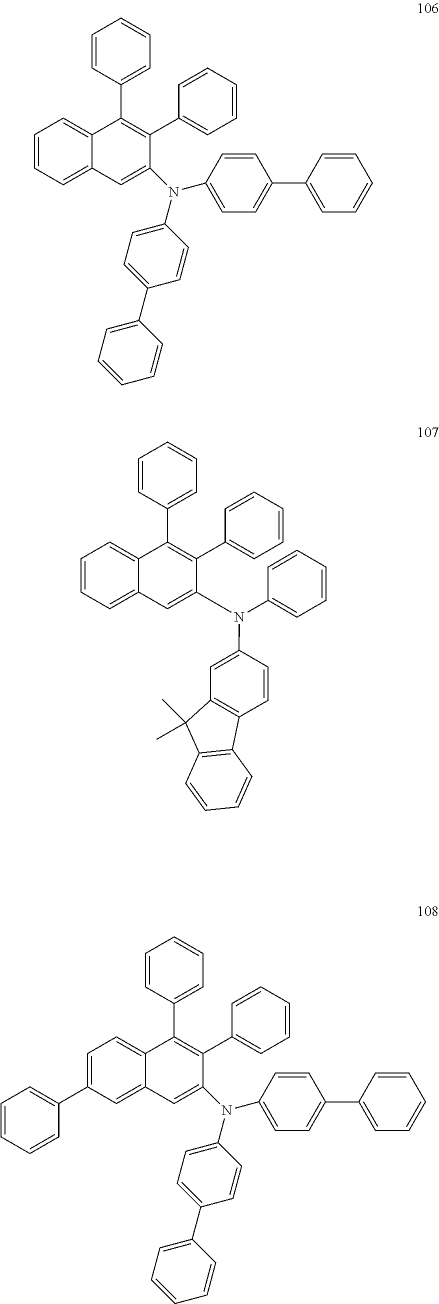 [SMILES: c1ccc(-c2ccc(N(c3ccc(-c4ccccc4)cc3)c3cc4ccccc4c(-c4ccccc4)c3-c3ccccc3)cc2)cc1 ; CC1(C)c2ccccc2-c2ccc(N(c3ccccc3)c3cc4ccccc4c(-c4ccccc4)c3-c3ccccc3)cc21 ; c1ccc(-c2ccc(N(c3ccc(-c4ccccc4)cc3)c3cc4cc(-c5ccccc5)ccc4c(-c4ccccc4)c3-c3ccccc3)cc2)cc1]

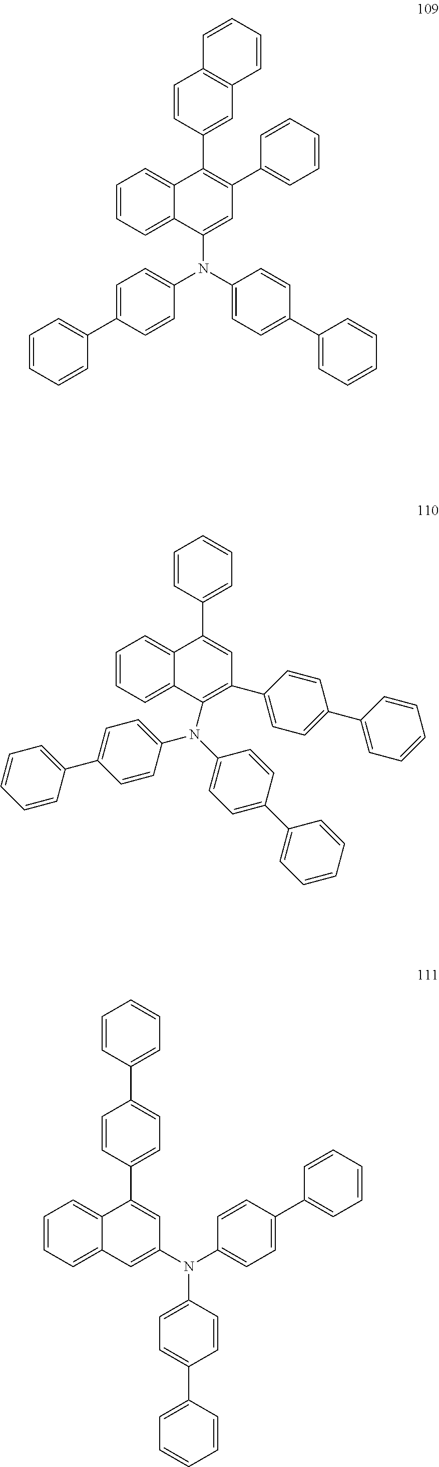 [SMILES: c1ccc(-c2ccc(N(c3ccc(-c4ccccc4)cc3)c3cc(-c4ccccc4)c(-c4ccc5ccccc5c4)c4ccccc34)cc2)cc1 ; c1ccc(-c2ccc(-c3cc(-c4ccccc4)c4ccccc4c3N(c3ccc(-c4ccccc4)cc3)c3ccc(-c4ccccc4)cc3)cc2)cc1 ; c1ccc(-c2ccc(-c3cc(N(c4ccc(-c5ccccc5)cc4)c4ccc(-c5ccccc5)cc4)cc4ccccc34)cc2)cc1]

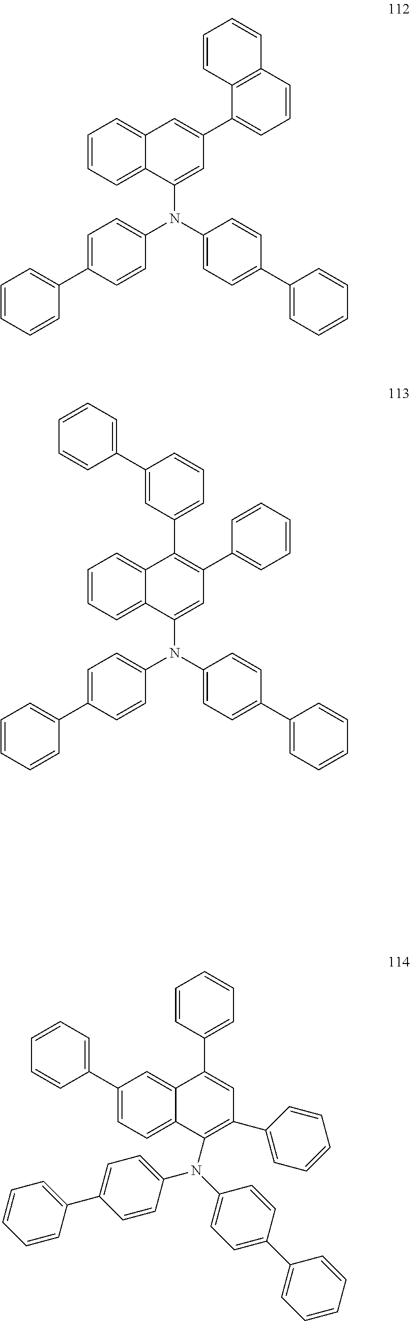 [SMILES: c1ccc(-c2ccc(N(c3ccc(-c4ccccc4)cc3)c3cc(-c4cccc5ccccc45)cc4ccccc34)cc2)cc1 ; c1ccc(-c2ccc(N(c3ccc(-c4ccccc4)cc3)c3cc(-c4ccccc4)c(-c4cccc(-c5ccccc5)c4)c4ccccc34)cc2)cc1 ; c1ccc(-c2ccc(N(c3ccc(-c4ccccc4)cc3)c3c(-c4ccccc4)cc(-c4ccccc4)c4cc(-c5ccccc5)ccc34)cc2)cc1]

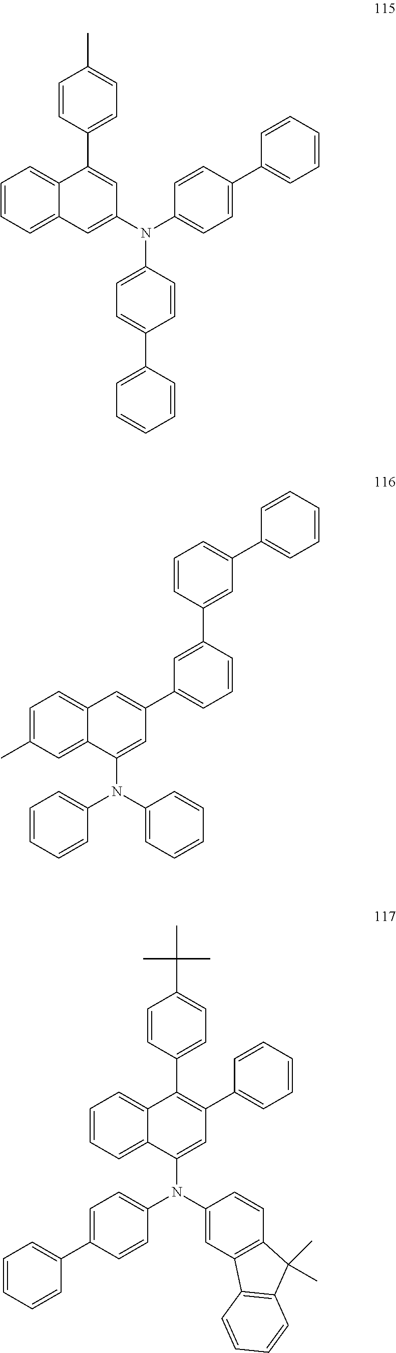 [SMILES: Cc1ccc(-c2cc(N(c3ccc(-c4ccccc4)cc3)c3ccc(-c4ccccc4)cc3)cc3ccccc23)cc1 ; Cc1ccc2cc(-c3cccc(-c4cccc(-c5ccccc5)c4)c3)cc(N(c3ccccc3)c3ccccc3)c2c1 ; CC(C)(C)c1ccc(-c2c(-c3ccccc3)cc(N(c3ccc(-c4ccccc4)cc3)c3ccc4c(c3)-c3ccccc3C4(C)C)c3ccccc23)cc1]

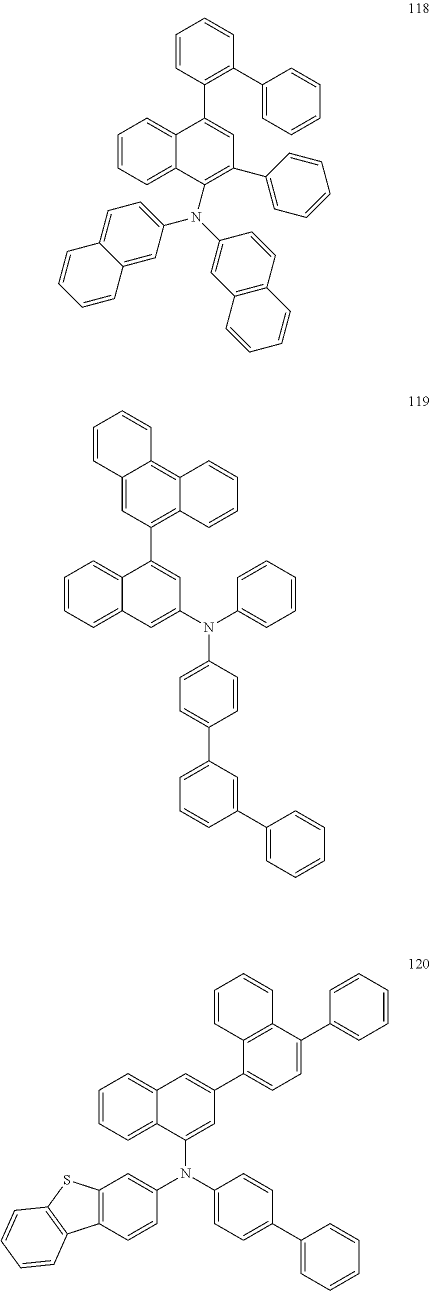 [SMILES: c1ccc(-c2ccccc2-c2cc(-c3ccccc3)c(N(c3ccc4ccccc4c3)c3ccc4ccccc4c3)c3ccccc23)cc1 ; c1ccc(-c2cccc(-c3ccc(N(c4ccccc4)c4cc(-c5cc6ccccc6c6ccccc56)c5ccccc5c4)cc3)c2)cc1 ; c1ccc(-c2ccc(N(c3ccc4c(c3)sc3ccccc34)c3cc(-c4ccc(-c5ccccc5)c5ccccc45)cc4ccccc34)cc2)cc1]

The specific examples of the substituents defined above can be found in Compounds 1 to 120 but are not intended to limit the scope of the compound represented by Formula A.

The introduction of the substituents on the naphthalene moiety allows the organic light emitting materials to have inherent characteristics of the substituents. For example, the introduced substituents may be those that are typically used in materials for hole injecting layers, hole transport layers, hole auxiliary layers, light emitting layers, electron transport layers, and electron injecting layers of organic light emitting diodes. This introduction meets the requirements of the organic layers. Particularly, a diode employing the compound of Formula A according to the present invention as a material for a hole auxiliary layer achieves further improved luminescent properties such as high luminous efficiency and long lifetime.

A further aspect of the present invention is directed to an organic light emitting diode including a first electrode, a second electrode, and one or more organic layers interposed between the first and second electrodes wherein one of the organic layers includes the organic light emitting compound represented by Formula A and optionally another organic light emitting compound represented by Formula A.

The organic light emitting diode can be fabricated by a suitable method known in the art using suitable materials known in the art, except that the organic light emitting compound of Formula A is used to form the corresponding organic layer.

The organic layers of the organic light emitting diode according to the present invention have a monolayer or multilayer structure. For example, the organic layers may be a hole injecting layer, a hole transport layer, a hole auxiliary layer, a light emitting layer, an electron transport layer, and an electron injecting layer. However, the number of the organic layers is not limited and may increase or decrease.

According to one embodiment of the present invention, the diode may include a substrate, a first electrode, a first hole injecting layer, a hole transport layer, a light emitting layer, an electron transport layer, an electron injecting layer, and a second electrode wherein a hole auxiliary layer including the compound represented by Formula A is interposed between the hole transport layer and the light emitting layer. The presence of the compound represented by Formula A facilitates hole transport to the light emitting layer, achieving further improved luminous efficiency and life characteristics of the diode.

A more detailed description will be given concerning embodiments of the organic light emitting diode according to the present invention.

The organic light emitting diode of the present invention includes an anode, a hole transport layer, a light emitting layer, an electron transport layer, and a cathode. The organic light emitting diode of the present invention may optionally further include a hole injecting layer between the anode and the hole transport layer and an electron injecting layer between the electron transport layer and the cathode. If necessary, the organic light emitting diode of the present invention may further include one or two intermediate layers. The intermediate layers may be a hole blocking layer or an electron blocking layer. The organic light emitting diode of the present invention may further include one or more organic layers that have various functions depending on the desired characteristics of the diode.

The organic light emitting diode of the present invention may further include a hole auxiliary layer between the hole transport layer and the light emitting layer wherein the hole auxiliary layer may include the compound represented by Formula A.

A specific structure of the organic light emitting diode according to the present invention and a method for fabricating the diode will be described below.

First, an electrode material for the anode is coated on the substrate to form the anode. The substrate may be any of those used in general organic light emitting diodes. The substrate is preferably an organic substrate or a transparent plastic substrate that is excellent in transparency, surface smoothness, ease of handling, and waterproofness. A highly transparent and conductive metal oxide, such as indium tin oxide (ITO), indium zinc oxide (IZO), tin oxide (SnO₂) or zinc oxide (ZnO), is used as the anode material.

A material for the hole injecting layer is coated on the anode by vacuum thermal evaporation or spin coating to form the hole injecting layer. Then, a material for the hole transport layer is coated on the hole injecting layer by vacuum thermal evaporation or spin coating to form the hole transport layer.

The material for the hole injecting layer is not specially limited so long as it is usually used in the art. Specific examples of such materials include 4,4′,4″-tris(2-naphthyl(phenyl)amino)triphenylamine (2-TNATA), N,N′-di(1-naphthyl)-N,N′-diphenylbenzidine (NPD), N,N′-diphenyl-N,N′-bis(3-methylphenyl)-1,1′-biphenyl-4,4′-diamine (TPD), and N,N′-diphenyl-N,N′-bis[4-(phenyl-m-tolylamino)phenyl]biphenyl-4,4′-diamine (DNTPD).

The material for the hole transport layer is not specially limited so long as it is commonly used in the art. Examples of such materials include N,N′-bis(3-methylphenyl)-N,N′-diphenyl-[1,1-biphenyl]-4,4′-diamine (TPD) and N,N′-di(naphthalen-1-yl)-N,N′-diphenylbenzidine (α-NPD).

Subsequently, the hole auxiliary layer and the light emitting layer are sequentially laminated on the hole transport layer. A hole blocking layer may be optionally formed on the organic light emitting layer by vacuum thermal evaporation or spin coating. The hole blocking layer blocks holes from entering the cathode through the organic light emitting layer. This role of the hole blocking layer prevents the lifetime and efficiency of the diode from deteriorating. A material having a very low highest occupied molecular orbital (HOMO) energy level is used for the hole blocking layer. The hole blocking material is not particularly limited so long as it has the ability to transport electrons and a higher ionization potential than the light emitting compound. Representative examples of suitable hole blocking materials include BAlq, BCP, and TPBI.

The electron transport layer is deposited on the hole blocking layer by vacuum thermal evaporation or spin coating, and the electron injecting layer is formed thereon. A metal for the cathode is deposited on the electron injecting layer by vacuum thermal evaporation to form the cathode, completing the fabrication of the organic light emitting diode.

As the metal for the cathode, there may be used, for example, lithium (Li), magnesium (Mg), aluminum (Al), aluminum-lithium (Al—Li), calcium (Ca), magnesium-indium (Mg—In) or magnesium-silver (Mg—Ag). The organic light emitting diode may be of top emission type. In this case, a transmissive material, such as ITO or IZO, may be used for the cathode.

The material for the electron transport layer functions to stably transport electrons injected from the cathode. The electron transport material may be any of those known in the art and examples thereof include, but are not limited to, quinoline derivatives, particularly, tris(8-quinolinolate)aluminum (Alq3), TAZ, Balq, beryllium bis(benzoquinolin-10-olate (Bebg2), ADN, and oxadiazole derivatives, such as PBD, BMD, and BND.

The light emitting layer may further include one or more light emitting dopant compounds. There is no particular restriction on the type of the dopant compounds applied to the organic light emitting diode of the present invention.

The light emitting layer may further include various host materials and various dopant materials in addition to the dopant compounds.

Each of the organic layers can be formed by a monomolecular deposition or solution process. According to the monomolecular deposition process, the material for each layer is evaporated under heat and vacuum or reduced pressure to form the layer in the form of a thin film. According to the solution process, the material for each layer is mixed with a suitable solvent, and then the mixture is formed into a thin film by a suitable method, such as ink-jet printing, roll-to-roll coating, screen printing, spray coating, dip coating or spin coating.

The organic light emitting diode of the present invention can be used in a display or lighting system selected from flat panel displays, flexible displays, monochromatic flat panel lighting systems, white flat panel lighting systems, flexible monochromatic lighting systems, and flexible white lighting systems.

The present invention will be explained in more detail with reference to the following examples. However, it will be obvious to those skilled in the art that these examples are in no way intended to limit the scope of the invention.

### Synthesis Example 1: Synthesis of Compound 1

### (1) Synthesis Example 1-(1): Synthesis of Intermediate 1-a

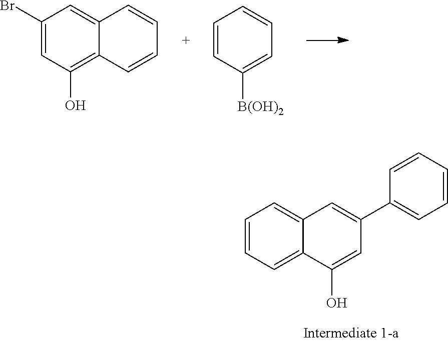 [SMILES: Oc1cc(Br)cc2ccccc12 ; OB(O)c1ccccc1 ; Oc1cc(-c2ccccc2)cc2ccccc12]

335 g (1.5 mol) of 3-bromo-1-naphthol, 246 g (2.0 mol) of phenylboronic acid, 33.5 g (31.0 mmol) of tetrakis(triphenylphosphine)palladium, 430 g (3.1 mol) of potassium carbonate, 2 L of toluene, 2 L of 1,4-dioxane, and 1 L of water were refluxed in a round-bottom flask for 16 h. After completion of the reaction, the reaction mixture was left standing for layer separation. The organic layer was concentrated under reduced pressure and recrystallized from toluene and methanol to afford 330 g of Intermediate 1-a (yield 75%).

### (2) Synthesis Example 1-(2): Synthesis of Intermediate 1-b

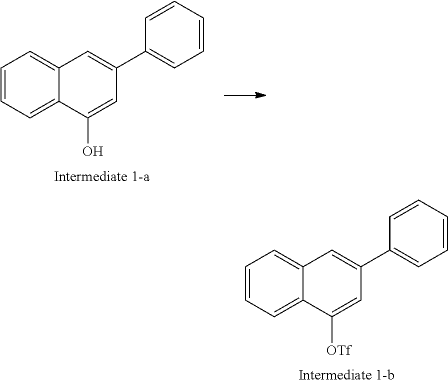 [SMILES: Oc1cc(-c2ccccc2)cc2ccccc12 ; O=S(=O)(Oc1cc(-c2ccccc2)cc2ccccc12)C(F)(F)F]

20 g (0.091 mol) of Intermediate 1-a was placed in a round-bottom flask and 200 mL of dichloromethane was added thereto. The mixture was stirred. The mixture was added with 10.8 g (0.136 mol) of pyridine and was cooled to 0° C. After slow dropwise addition of 38.4 g (0.136 mol) of trifluoromethanesulfonic anhydride, the resulting mixture was heated to room temperature, followed by stirring for 3 h. The reaction mixture was poured into water (600 mL). The organic layer was extracted, concentrated under reduced pressure, and purified by column chromatography to afford 31 g of Intermediate 1-b (yield 97%).

### (3) Synthesis Example 1-(3): Synthesis of Compound 1

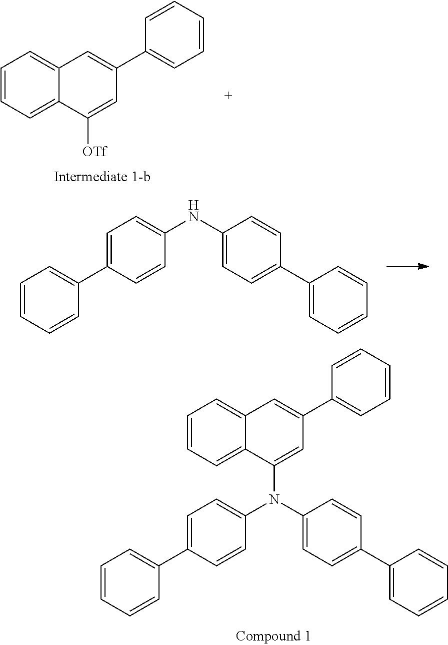

9.6 g (0.027 mol) of Intermediate 1-b, 5.5 g (0.017 mol) of bis(biphenyl-4-yl)amine, 0.6 g (0.0007 mol) of tris(dibenzylideneacetone)dipalladium, 1.3 g (0.0014 mol) of 2-dicyclohexylphosphino-2′-(N,N-dimethylamino)biphenyl, 3.3 g (0.034 mol) of sodium butoxide, and 55 mL of toluene were added to a round-bottom flask under a nitrogen atmosphere. The mixture was refluxed for 3 h. After completion of the reaction, the reaction mixture was extracted. The organic layer was concentrated under reduced pressure, purified by column chromatography, and dried to give 7.0 g of Compound 1 (yield 78%)

MS (MALDI-TOF): m/z 523.23[M⁺]

### Synthesis Example 2: Synthesis of Compound 7

### (1) Synthesis Example 2-(1): Synthesis of Intermediate 2-a

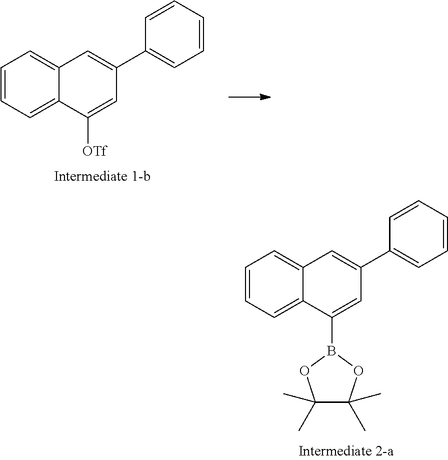 [SMILES: O=S(=O)(Oc1cc(-c2ccccc2)cc2ccccc12)C(F)(F)F ; CC1(C)OB(c2cc(-c3ccccc3)cc3ccccc23)OC1(C)C]

20 g (0.057 mol) of Intermediate 1-b, 21.6 g (0.085 mol) of bis(pinacolato)diborane, 1.2 g (0.002 mol) of [1,1′-bis(diphenylphosphino)ferrocene]palladium(II) dichloride, 16.7 g (0.17 mol) of potassium acetate, and 200 mL of toluene were added to a round-bottom flask under a nitrogen atmosphere. The mixture was refluxed. After completion of the reaction, the reaction mixture was left standing for layer separation. The organic layer was concentrated under reduced pressure, purified by column chromatography, and dried to afford 15 g of Intermediate 2-a (yield 80%)

### (2) Synthesis Example 2-(2): Synthesis of Intermediate 2-b

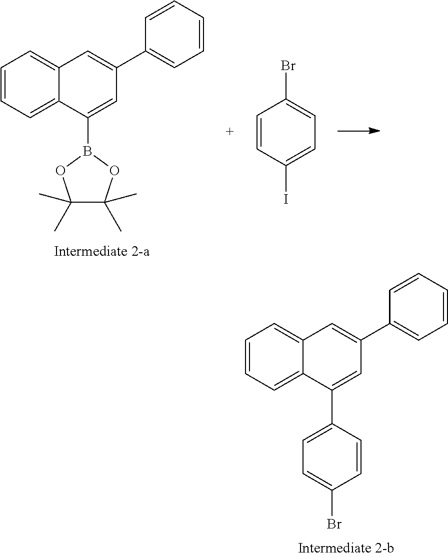 [SMILES: CC1(C)OB(c2cc(-c3ccccc3)cc3ccccc23)OC1(C)C ; Brc1ccc(I)cc1 ; Brc1ccc(-c2cc(-c3ccccc3)cc3ccccc23)cc1]

Intermediate 2-b was synthesized in the same manner as in Synthesis Example 1-(1), except that 1,4-bromoiodobenzene and Intermediate 2-a were used instead of 3-bromo-1-naphthol and phenylboronic acid, respectively.

### (3) Synthesis Example 2-(3): Synthesis of Compound 7

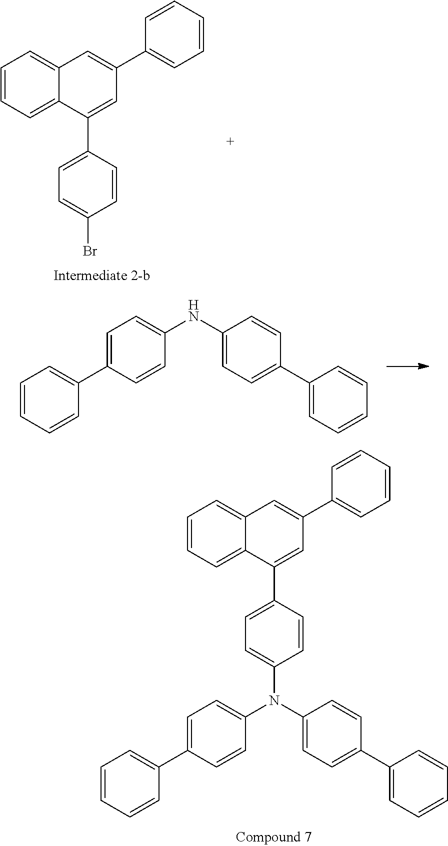

6.0 g (0.017 mol) of Intermediate 2-b, 5.9 g (0.018 mol) of bis(biphenyl-4-yl)amine, 0.3 g (0.0003 mol) of tris(dibenzylideneacetone)dipalladium, 0.2 g (0.0003 mol) of tri-tert-butylphosphine, 3.2 g (0.033 mol) of sodium butoxide, and 60 mL of toluene were added to a round-bottom flask under a nitrogen atmosphere. The mixture was refluxed for 12 h. After completion of the reaction, the reaction mixture was extracted. The organic layer was concentrated under reduced pressure, purified by column chromatography, and dried to give 7.2 g of Compound 7 (yield 72%).

MS (MALDI-TOF): m/z 599.26[M*]

### Synthesis Example 3: Synthesis of Compound 14

### (1) Synthesis Example 3-(1): Synthesis of Intermediate 3-a

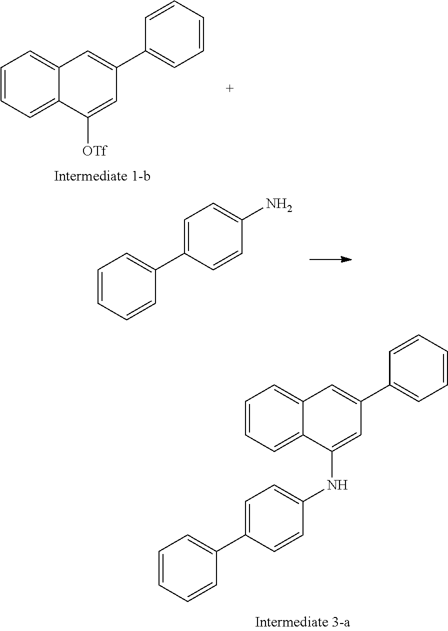 [SMILES: O=S(=O)(Oc1cc(-c2ccccc2)cc2ccccc12)C(F)(F)F ; Nc1ccc(-c2ccccc2)cc1 ; c1ccc(-c2ccc(Nc3cc(-c4ccccc4)cc4ccccc34)cc2)cc1]

Intermediate 3-a was synthesized in the same manner as in Synthesis Example 1-(3), except that 4-aminobiphenyl was used instead of bis(biphenyl-4-yl)amine.

### (2) Synthesis Example 3-(2): Synthesis of Compound 14

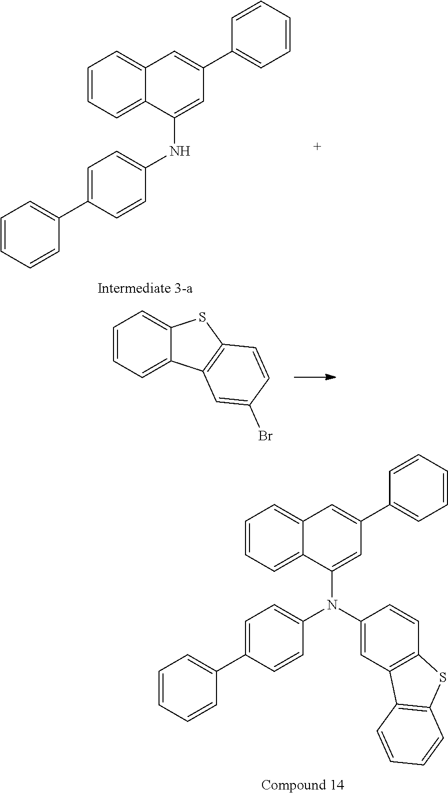 [SMILES: c1ccc(-c2ccc(Nc3cc(-c4ccccc4)cc4ccccc34)cc2)cc1 ; Brc1ccc2sc3ccccc3c2c1 ; c1ccc(-c2ccc(N(c3ccc4sc5ccccc5c4c3)c3cc(-c4ccccc4)cc4ccccc34)cc2)cc1]

Compound 14 was synthesized in the same manner as in Synthesis Example 2-(3), except that Intermediate 3-1 and 2-bromodibenzothiophene were used instead of Intermediate 2-b and bis(biphenyl-4-yl)amine, respectively.

MS (MALDI-TOF): m/z 533.19[M⁺]

### Synthesis Example 4: Synthesis of Compound 28

### (1) Synthesis Example 4-(1): Synthesis of Intermediate 4-a

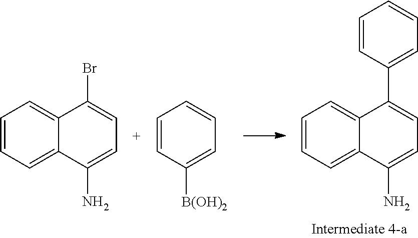

Intermediate 4-1 was synthesized in the same manner as in Synthesis Example 1-(1), except that 4-bromo-1-naphthylamine was used instead of 3-bromo-1-naphthol.

### (2) Synthesis Example 4-(2): Synthesis of Intermediate 4-b

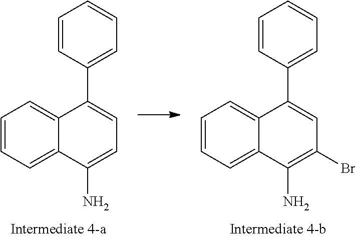 [SMILES: Nc1ccc(-c2ccccc2)c2ccccc12 ; Nc1c(Br)cc(-c2ccccc2)c2ccccc12]

42 g (0.192 mol) of Intermediate 4-a was added to a round-bottom flask and 300 mL of dimethylformamide was added thereto. The mixture was stirred. To the mixture was added dropwise a solution of 34.1 g (0.192 mol) of N-bromosuccinimide in 120 mL of dimethylformamide at 0° C. The resulting mixture was stirred at room temperature for 12 h. After completion of the reaction, the reaction mixture was poured into water (1000 mL), followed by filtration. The collected solid was purified by column chromatography and dried to afford 36 g of Intermediate 4-b (yield 63%).

### (3) Synthesis Example 4-(3): Synthesis of Intermediate 4-c

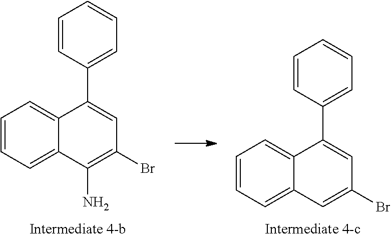 [SMILES: Nc1c(Br)cc(-c2ccccc2)c2ccccc12 ; Brc1cc(-c2ccccc2)c2ccccc2c1]

36 g (0.121 mol) of Intermediate 4-b was placed in a round-bottom flask and 540 mL of tetrahydrofuran was added thereto. The mixture was stirred. To the mixture was added dropwise 159.4 g (2.415 mol) of hypophosphorous acid at 0° C. After addition of 25 g (0.362 mol) of sodium nitrate, stirring was continued for 4 h. The temperature was allowed to rise to room temperature. The resulting mixture was stirred for 12 h. After completion of the reaction, dichloromethane and water were added. The mixture was adjusted to a pH of 10 with 2 N NaOH. The organic layer was extracted, purified by column chromatography, and dried to afford 18 g of Intermediate 4-c (yield 53%).

### (4) Synthesis Example 4-(4): Synthesis of Intermediate 4-d

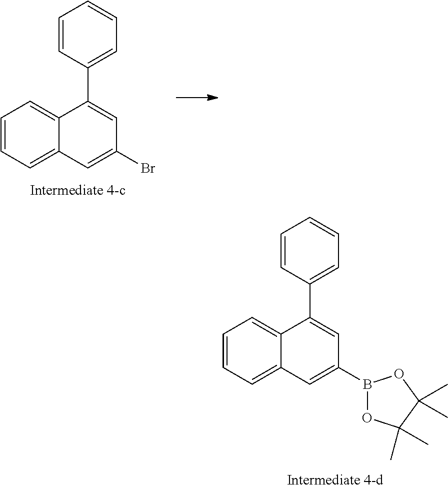 [SMILES: Brc1cc(-c2ccccc2)c2ccccc2c1 ; CC1(C)OB(c2cc(-c3ccccc3)c3ccccc3c2)OC1(C)C]

Intermediate 4-d was synthesized in the same manner as in Synthesis Example 2-(1), except that Intermediate 4-c was used instead of Intermediate 1-b.

### (5) Synthesis Example 4-(5): Synthesis of Compound 28

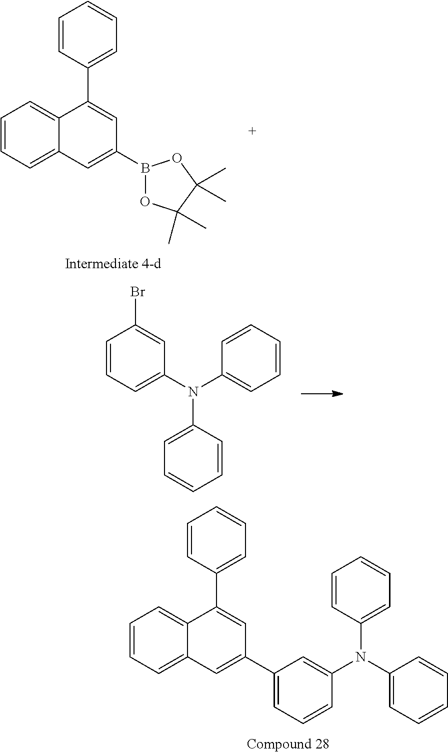

Compound 28 was synthesized in the same manner as in Synthesis Example 1-(1), except that N-3-bromophenyl-N,N-diphenylamine and Intermediate 4-d were used instead of 3-bromo-1-naphthol and phenylboronic acid, respectively.

MS (MALDI-TOF): m/z 447.20[M*]

### Synthesis Example 5: Synthesis of Compound 29

### Synthesis Example 5-(1): Synthesis of Compound 29

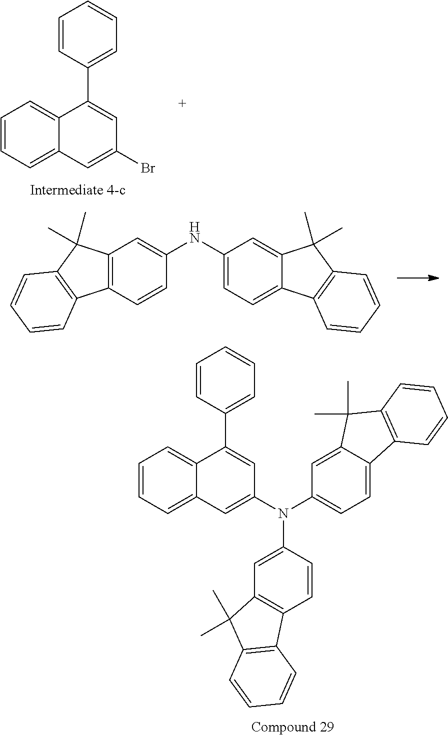

Compound 29 was synthesized in the same manner as in Synthesis Example 2-(3), except that Intermediate 4-c and bis(9,9′-dimethyl-9H-fluoren-2-yl)amine were used instead of Intermediate 2-b and bis(biphenyl-4-yl)amine, respectively.

MS (MALDI-TOF): m/z 603.29[M*]

### Synthesis Example 6: Synthesis of Compound 37

### (1) Synthesis Example 6-(1): Synthesis of Intermediate 6-a

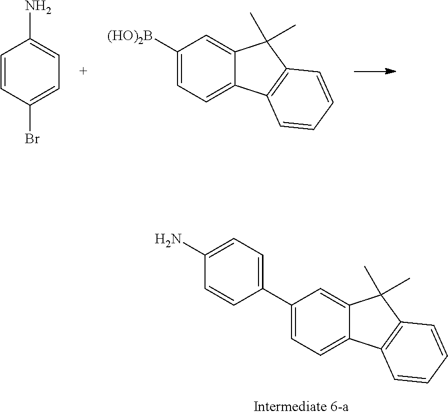 [SMILES: Nc1ccc(Br)cc1 ; CC1(C)c2ccccc2-c2ccc(B(O)O)cc21 ; CC1(C)c2ccccc2-c2ccc(-c3ccc(N)cc3)cc21]

Intermediate 6-a was synthesized in the same manner as in Synthesis Example 1-(1), except that 4-bromoaniline and 9,9′-dimethyl-9H-fluoren-2-yl-2-boronic acid were used instead of 3-bromo-1-naphthol and phenylboronic acid, respectively.

### (2) Synthesis Example 6-(2): Synthesis of Intermediate 6-b

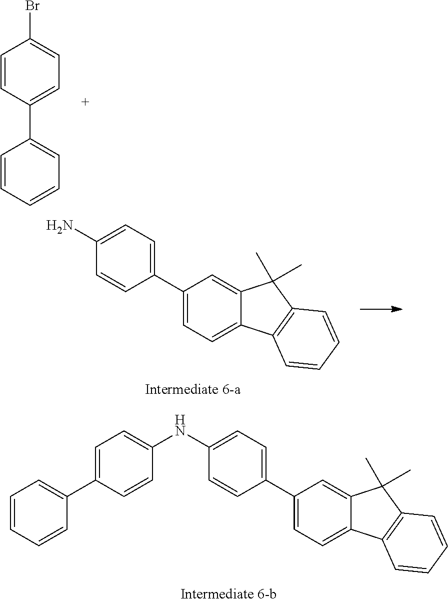

Intermediate 6-b was synthesized in the same manner as in Synthesis Example 2-(3), except that 4-bromo-1,1′-biphenyl and Intermediate 6-a were used instead of Intermediate 2-b and bis(biphenyl-4-yl)amine, respectively.

### (3) Synthesis Example 6-(3): Synthesis of Compound 37

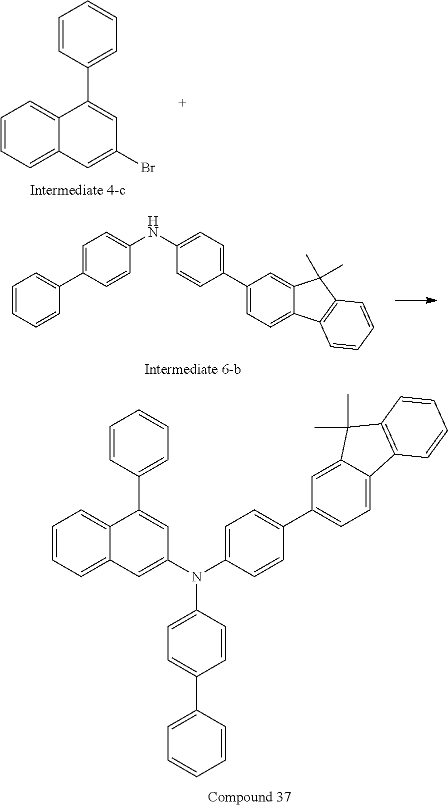

Compound 37 was synthesized in the same manner as in Synthesis Example 2-(3), except that Intermediate 4-c and Intermediate 6-b were used instead of Intermediate 2-b and bis(biphenyl-4-yl)amine, respectively.

MS (MALDI-TOF): m/z 639.29[M⁺]

### Synthesis Example 7: Synthesis of Compound 53

### (1) Synthesis Example 7-(1): Synthesis of Intermediate 7-a

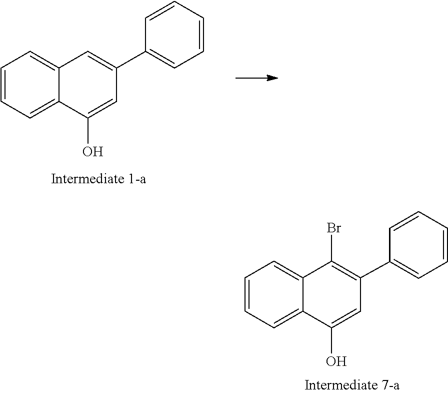 [SMILES: Oc1cc(-c2ccccc2)cc2ccccc12 ; Oc1cc(-c2ccccc2)c(Br)c2ccccc12]

Intermediate 7-a was synthesized in the same manner as in Synthesis Example 4-(2), except that Intermediate 1-a was used instead of Intermediate 4-a.

### (2) Synthesis Example 7-(2): Synthesis of Intermediate 7-b

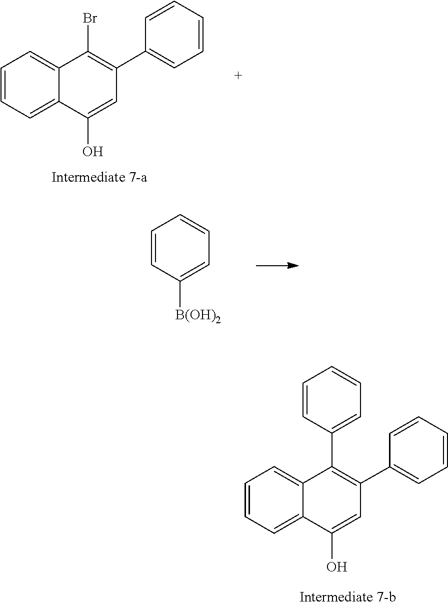 [SMILES: Oc1cc(-c2ccccc2)c(Br)c2ccccc12 ; OB(O)c1ccccc1 ; Oc1cc(-c2ccccc2)c(-c2ccccc2)c2ccccc12]

Intermediate 7-b was synthesized in the same manner as in Synthesis Example 1-(1), except that Intermediate 7-a was used instead of 3-bromo-1-naphthol.

### (3) Synthesis Example 7-(3): Synthesis of Intermediate 7-c

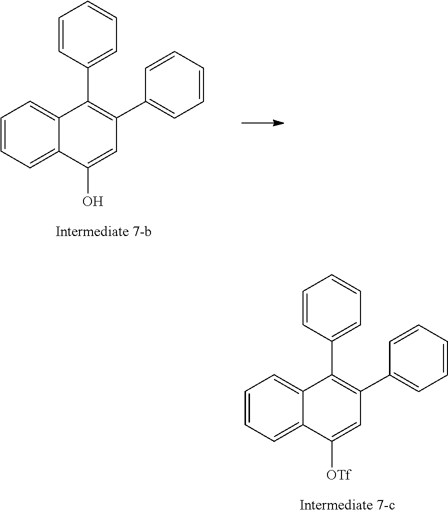 [SMILES: Oc1cc(-c2ccccc2)c(-c2ccccc2)c2ccccc12 ; O=S(=O)(Oc1cc(-c2ccccc2)c(-c2ccccc2)c2ccccc12)C(F)(F)F]

Intermediate 7-c was synthesized in the same manner as in Synthesis Example 1-(2), except that Intermediate 7-b was used instead of Intermediate 1-a.

### (4) Synthesis Example 7-(4): Synthesis of Compound 53

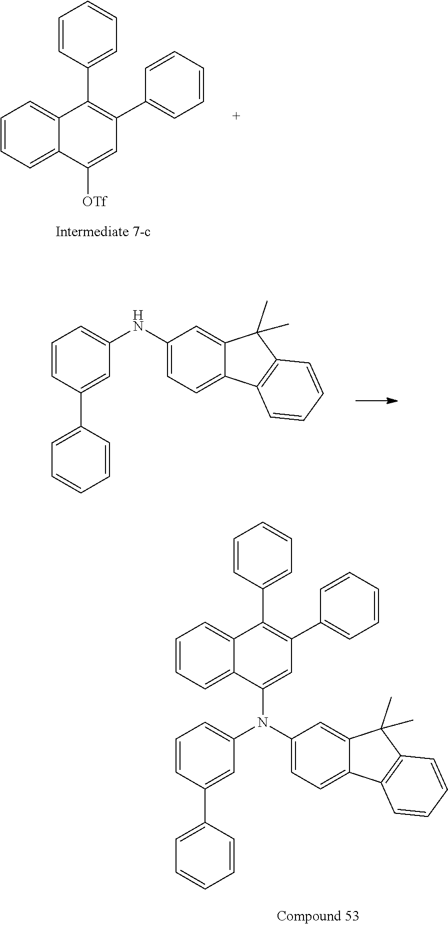 [SMILES: O=S(=O)(Oc1cc(-c2ccccc2)c(-c2ccccc2)c2ccccc12)C(F)(F)F ; CC1(C)c2ccccc2-c2ccc(Nc3cccc(-c4ccccc4)c3)cc21 ; CC1(C)c2ccccc2-c2ccc(N(c3cccc(-c4ccccc4)c3)c3cc(-c4ccccc4)c(-c4ccccc4)c4ccccc34)cc21]

Compound 53 was synthesized in the same manner as in Synthesis Example 1-(3), except that Intermediate 7-c and N-((1,1′-biphenyl)-3-yl)-9,9-dimethyl-9H-fluoren-2-amine were used instead of Intermediate 1-b and bis(biphenyl-4-yl)amine, respectively.

MS (MALDI-TOF): m/z 639.29[M⁺]

### Synthesis Example 8: Synthesis of Compound 56

### (1) Synthesis Example 8-(1): Synthesis of Intermediate 8-a

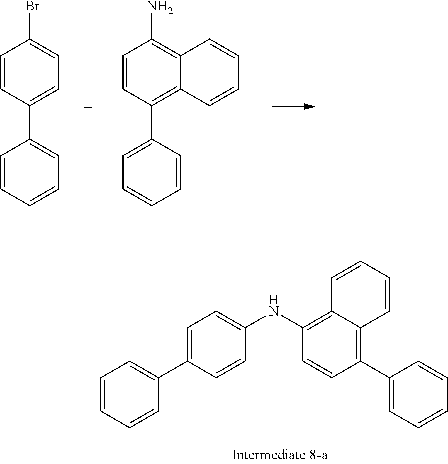 [SMILES: Brc1ccc(-c2ccccc2)cc1 ; Nc1ccc(-c2ccccc2)c2ccccc12 ; c1ccc(-c2ccc(Nc3ccc(-c4ccccc4)c4ccccc34)cc2)cc1]

Intermediate 8-a was synthesized in the same manner as in Synthesis Example 2-(3), except that 1-amino-4-phenylnaphthalene and 4-bromo-1,1′-biphenyl were used instead of bis(biphenyl-4-yl)amine and Intermediate 2-b, respectively.

### (2) Synthesis Example 8-(2): Synthesis of Compound 56

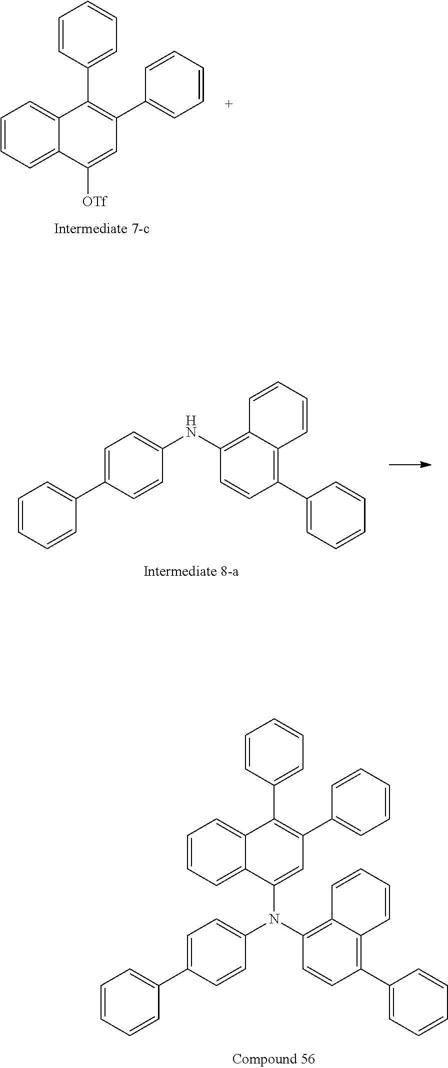 [SMILES: O=S(=O)(Oc1cc(-c2ccccc2)c(-c2ccccc2)c2ccccc12)C(F)(F)F ; c1ccc(-c2ccc(Nc3ccc(-c4ccccc4)c4ccccc34)cc2)cc1 ; c1ccc(-c2ccc(N(c3ccc(-c4ccccc4)c4ccccc34)c3cc(-c4ccccc4)c(-c4ccccc4)c4ccccc34)cc2)cc1]

Compound 56 was synthesized in the same manner as in Synthesis Example 1-(3), except that Intermediate 7-c and Intermediate 8-a were used instead of Intermediate 1-b and bis(biphenyl-4-yl)amine, respectively.

MS (MALDI-TOF): m/z 649.28[M*]

### Synthesis Example 9: Synthesis of Compound 63

### (1) Synthesis Example 9-(1): Synthesis of Compound 63

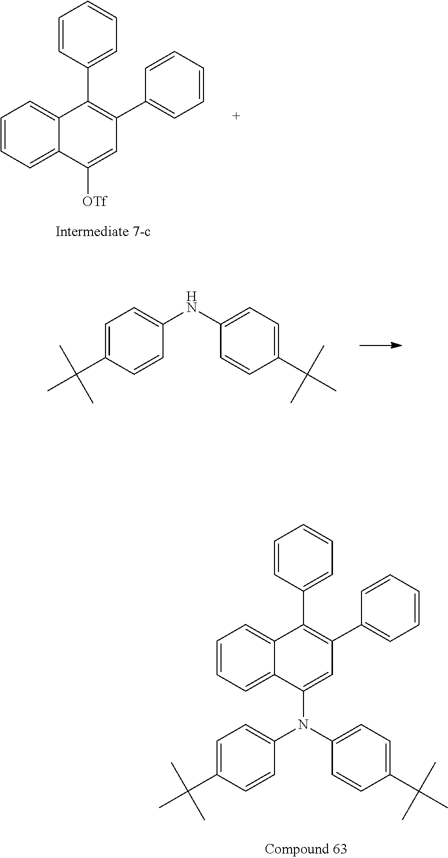

Compound 63 was synthesized in the same manner as in Synthesis Example 1-(3), except that Intermediate 7-c and bis(4-tert-butylphenyl)amine were used instead of Intermediate 1-b and bis(biphenyl-4-yl)amine, respectively.

MS (MALDI-TOF): m/z 559.32[M*]

### Synthesis Example 10: Synthesis of Compound 66

### Synthesis Example 10-(1): Synthesis of Intermediate 10-a

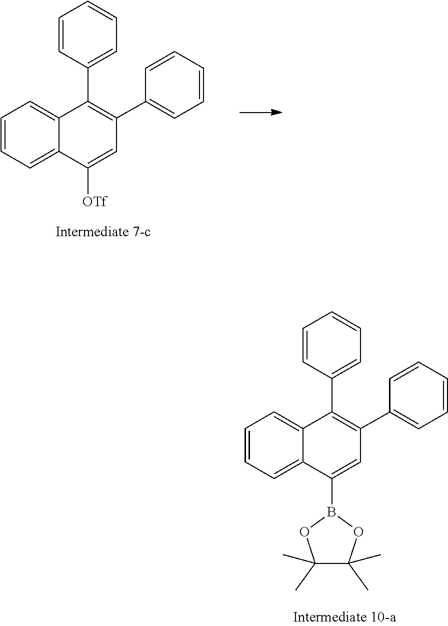 [SMILES: O=S(=O)(Oc1cc(-c2ccccc2)c(-c2ccccc2)c2ccccc12)C(F)(F)F ; CC1(C)OB(c2cc(-c3ccccc3)c(-c3ccccc3)c3ccccc23)OC1(C)C]

Intermediate 10-a was synthesized in the same manner as in Synthesis Example 2-(1), except that Intermediate 7-c was used instead of Intermediate 1-b.

### (2) Synthesis Example 10-(2): Synthesis of Intermediate 10-b

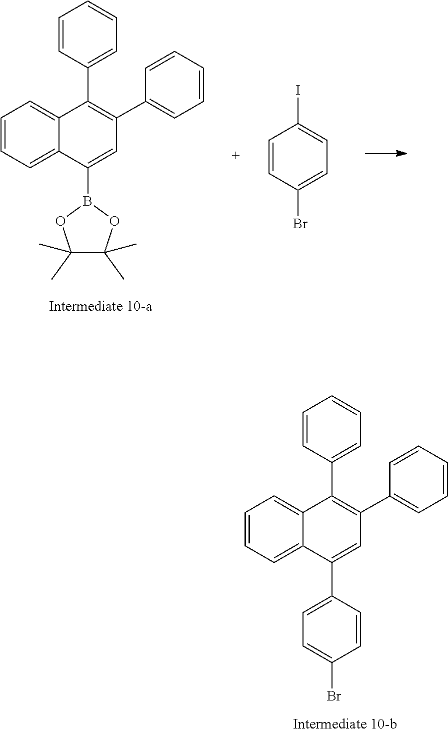 [SMILES: CC1(C)OB(c2cc(-c3ccccc3)c(-c3ccccc3)c3ccccc23)OC1(C)C ; Brc1ccc(I)cc1 ; Brc1ccc(-c2cc(-c3ccccc3)c(-c3ccccc3)c3ccccc23)cc1]

Intermediate 10-b was synthesized in the same manner as in Synthesis Example 1-(1), except that 1,4-bromoiodobenzene and Intermediate 10-a were used instead of 3-bromo-1-naphthol and phenylboronic acid, respectively.

### (3) Synthesis Example 10-(3): Synthesis of Intermediate 10-c

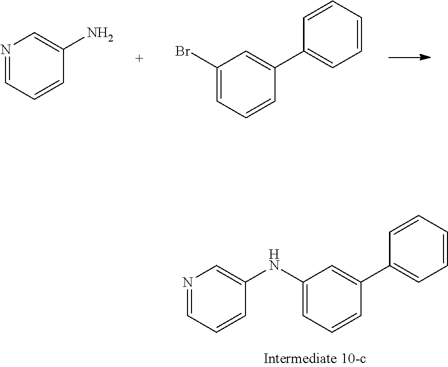 [SMILES: Nc1cccnc1 ; Brc1cccc(-c2ccccc2)c1 ; c1ccc(-c2cccc(Nc3cccnc3)c2)cc1]

Intermediate 10-c was synthesized in the same manner as in Synthesis Example 2-(3), except that pyridin-3-ylamine and 3-bromobiphenyl were used instead of bis(biphenyl-4-yl)amine and Intermediate 2-b, respectively.

### (4) Synthesis Example 10-(4): Synthesis of Compound 66

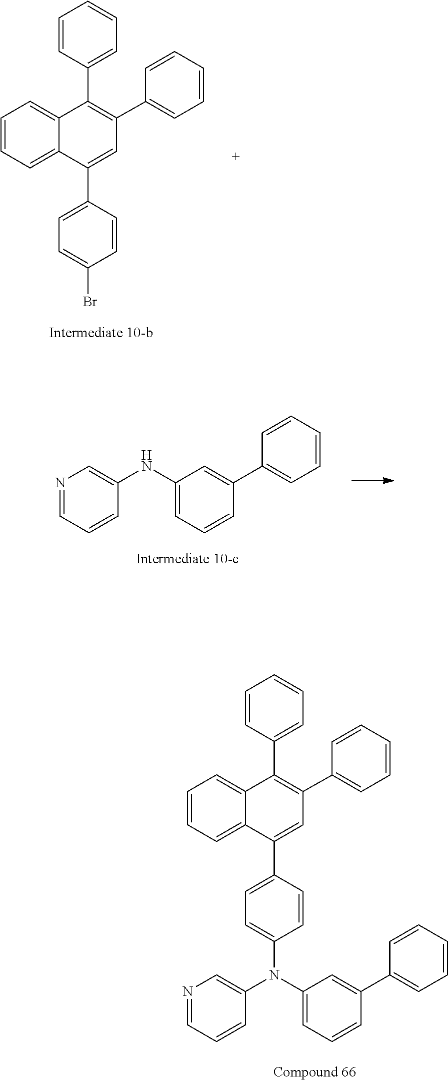 [SMILES: Brc1ccc(-c2cc(-c3ccccc3)c(-c3ccccc3)c3ccccc23)cc1 ; c1ccc(-c2cccc(Nc3cccnc3)c2)cc1 ; c1ccc(-c2cccc(N(c3ccc(-c4cc(-c5ccccc5)c(-c5ccccc5)c5ccccc45)cc3)c3cccnc3)c2)cc1]

Compound 66 was synthesized in the same manner as in Synthesis Example 2-(3), except that Intermediate 10-b and Intermediate 10-c were used instead of Intermediate 2-b and bis(biphenyl-4-yl)amine, respectively.

MS (MALDI-TOF): m/z 600.26[M*]

### Synthesis Example 11: Synthesis of Compound 106

### (1) Synthesis Example 11-(1): Synthesis of Intermediate 11-a

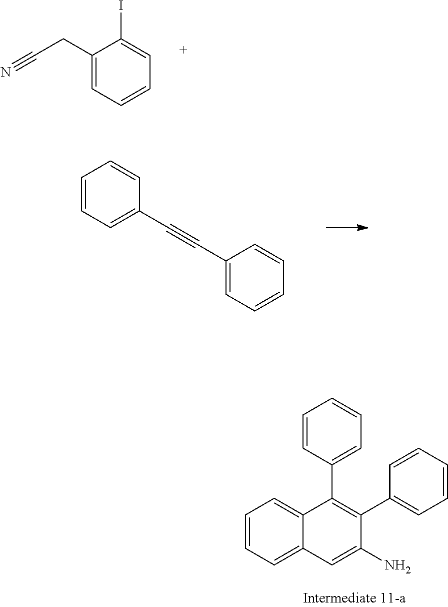

30.0 g (0.123 mol) of 2-iodophenylacetonitrile, 66.0 g (0.370 mol) of diphenylacetylene, 11.3 g (0.0123 mol) of tris(dibenzylideneacetone)dipalladium, 30.0 g (0.123 mol) of triethylamine, 210 mL of dimethylformamide, and 30 mL of water were stirred in a round-bottom flask at 130° C. for 48 h. After completion of the reaction, the organic layer was extracted, purified by column chromatography, and dried to afford 28 g of Intermediate 11-a (yield 77%).

### (2) Synthesis Example 11-(2): Synthesis of Compound 106

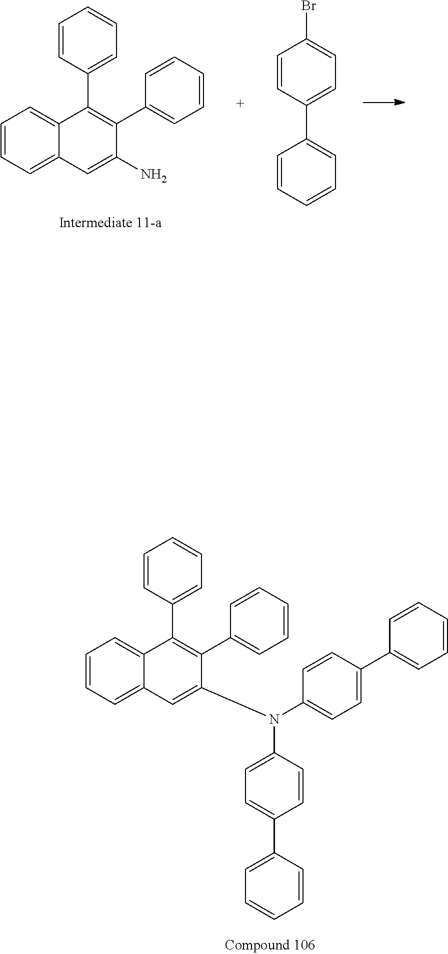 [SMILES: Nc1cc2ccccc2c(-c2ccccc2)c1-c1ccccc1 ; Brc1ccc(-c2ccccc2)cc1 ; c1ccc(-c2ccc(N(c3ccc(-c4ccccc4)cc3)c3cc4ccccc4c(-c4ccccc4)c3-c3ccccc3)cc2)cc1]

6.0 g (0.020 mol) of Intermediate 11-a, 11.8 g (0.051 mol) of 4-bromobiphenyl, 0.4 g (0.0004 mol) of tris(dibenzylideneacetone)dipalladium, 0.2 g (0.0004 mol) of tri-tert-butylphosphine, 3.9 g (0.041 mol) of STB, and 60 mL of toluene were added to a round-bottom flask under a nitrogen atmosphere. The mixture was refluxed for 12 h. After completion of the reaction, the reaction mixture was extracted. The organic layer was concentrated under reduced pressure, concentrated under reduced pressure, and dried to give 7.4 g of Compound 106 (yield 61%).

MS (MALDI-TOF): m/z 599.26[M⁺]

### Synthesis Example 12: Synthesis of Compound 109

### (1) Synthesis Example 12-(1): Synthesis of Intermediate 12-a

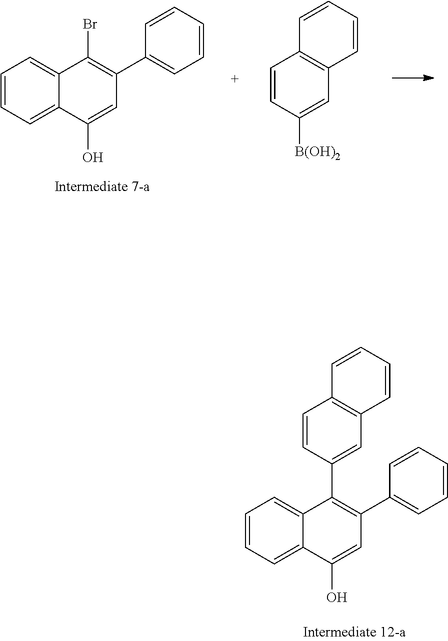 [SMILES: Oc1cc(-c2ccccc2)c(Br)c2ccccc12 ; OB(O)c1ccc2ccccc2c1 ; Oc1cc(-c2ccccc2)c(-c2ccc3ccccc3c2)c2ccccc12]

Intermediate 12-a was synthesized in the same manner as in Synthesis Example 1-(1), except that Intermediate 7-a and 2-naphthaleneboronic acid were used instead of 3-bromo-1-naphthol and phenylboronic acid, respectively.

### (2) Synthesis Example 12-(2): Synthesis of Intermediate 12-b

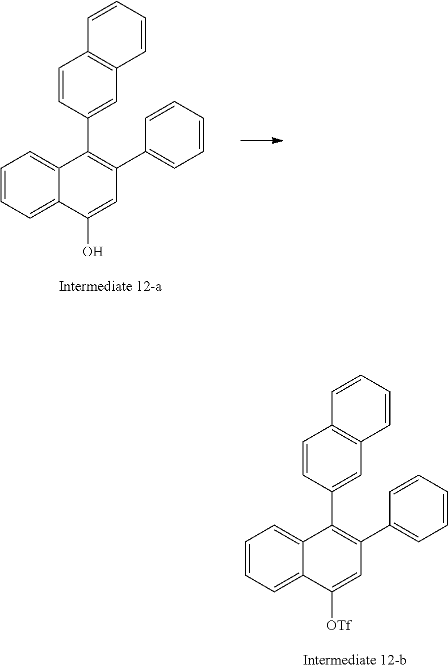 [SMILES: Oc1cc(-c2ccccc2)c(-c2ccc3ccccc3c2)c2ccccc12 ; O=S(=O)(Oc1cc(-c2ccccc2)c(-c2ccc3ccccc3c2)c2ccccc12)C(F)(F)F]

Intermediate 12-b was synthesized in the same manner as in Synthesis Example 1-(2), except that Intermediate 12-a was used instead of Intermediate 1-a.

### (3) Synthesis Example 12-(3): Synthesis of Compound 109

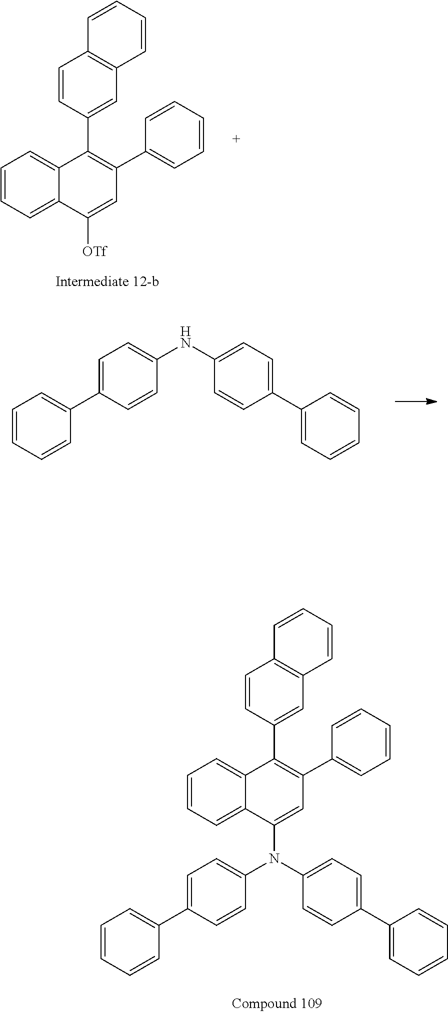 [SMILES: O=S(=O)(Oc1cc(-c2ccccc2)c(-c2ccc3ccccc3c2)c2ccccc12)C(F)(F)F ; c1ccc(-c2ccc(Nc3ccc(-c4ccccc4)cc3)cc2)cc1 ; c1ccc(-c2ccc(N(c3ccc(-c4ccccc4)cc3)c3cc(-c4ccccc4)c(-c4ccc5ccccc5c4)c4ccccc34)cc2)cc1]

Compound 109 was synthesized in the same manner as in Synthesis Example 1-(3), except that Intermediate 12-b was used instead of Intermediate 1-b.

MS (MALDI-TOF): m/z 649.28[M⁺]

### Examples 1 to 16: Fabrication of Blue Organic Light Emitting Diodes

ITO glass was patterned to have a light emitting area of 2 mm×2 mm, followed by cleaning. After the cleaned ITO glass was mounted in a vacuum chamber, the base pressure was adjusted to 1×10⁻⁶ torr. HATCN (50 Å) and NPD (650 Å) were deposited in this order on the ITO glass. The corresponding amine compound shown in Table 1 was formed on the NPD layer to form a hole auxiliary layer (50 Å). The hole auxiliary layer was doped with Blue host (BH)+ Blue dopant (BD) 5% to form a 200 Å thick light emitting layer. Thereafter, [ET]:Liq (1:1) (300 Å), Liq (10 Å), and Al (1,000 Å) were deposited in this order on the light emitting layer to fabricate an organic light emitting diode. The luminescent properties of the organic light emitting diode were measured at 0.4 mA. The structures of [HATCN], [NPD], [BH], [BD],_[Liq], and [ET] are as follows:

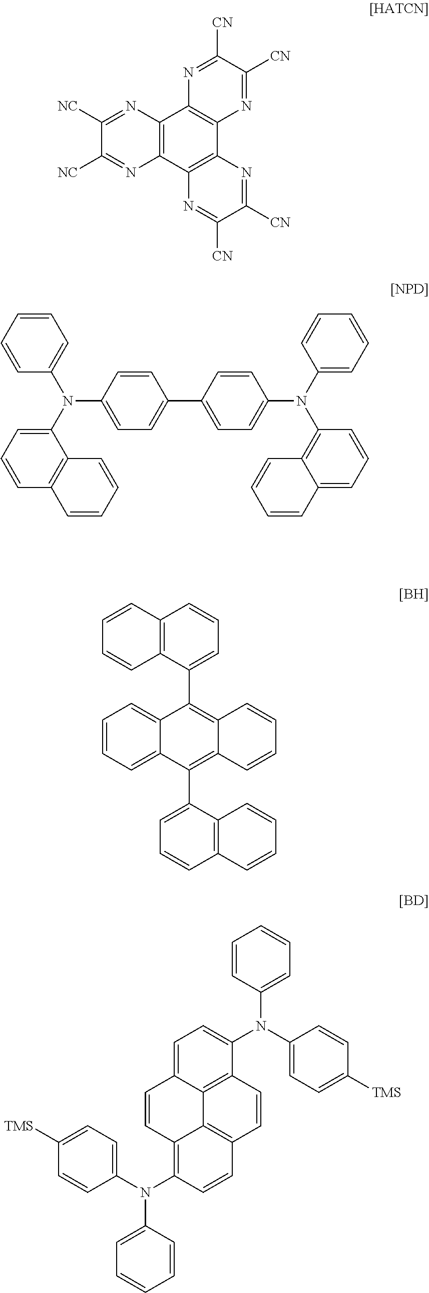

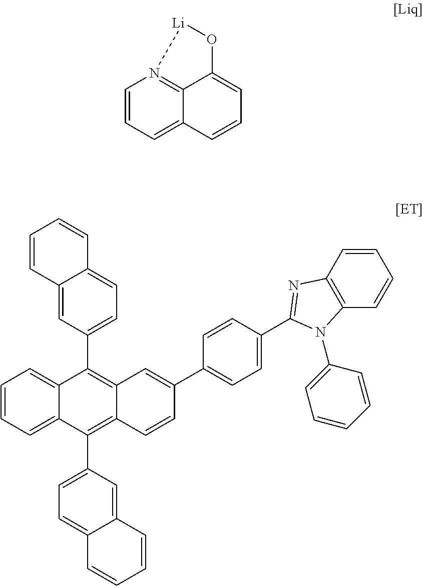

### Comparative Example 1

An organic light emitting diode was fabricated in the same manner as in Examples 1-16, except that the hole auxiliary layer was not formed and the thickness of the hole transport layer employing NPD was changed to 650 Å.

The organic light emitting diodes of Examples 1-16 and Comparative Example 1 were measured for voltage, current, luminance, color coordinates, and lifetime. The results are shown in Table 1. T₉₅ indicates the time at which the luminance of each diode was decreased to 95% of the initial luminance (2000 cd/m²).

TABLE 1

| | Hole auxiliary | | | | | T₉₅ |
|---|---|---|---|---|---|---|
| Example No. | layer | V | Cd/A | CIEx | CIEy | (Hrs) |
| Comparative Example 1 | — | 4.2 | 6.7 | 0.133 | 0.129 | 16 |
| Example 1 | Compound 1 | 4.1 | 8.4 | 0.133 | 0.129 | 32 |
| Example 2 | Compound 7 | 4.1 | 8.3 | 0.133 | 0.128 | 29 |
| Example 3 | Compound 14 | 4.2 | 8.0 | 0.133 | 0.129 | 27 |
| Example 4 | Compound 23 | 4.1 | 8.0 | 0.133 | 0.129 | 26 |
| Example 5 | Compound 28 | 4.1 | 8.1 | 0.135 | 0.128 | 31 |
| Example 6 | Compound 29 | 4.2 | 8.4 | 0.134 | 0.130 | 30 |
| Example 7 | Compound 37 | 4.2 | 8.3 | 0.134 | 0.129 | 29 |
| Example 8 | Compound 53 | 4.2 | 8.2 | 0.133 | 0.129 | 28 |
| Example 9 | Compound 56 | 4.2 | 8.2 | 0.133 | 0.128 | 26 |
| Example 10 | Compound 63 | 4.1 | 8.1 | 0.134 | 0.128 | 29 |
| Example 11 | Compound 66 | 4.1 | 8.0 | 0.133 | 0.129 | 25 |
| Example 12 | Compound 70 | 4.2 | 8.1 | 0.134 | 0.129 | 25 |
| Example 13 | Compound 92 | 4.2 | 8.1 | 0.134 | 0.128 | 26 |
| Example 14 | Compound 106 | 4.2 | 8.4 | 0.134 | 0128 | 27 |
| Example 15 | Compound 109 | 4.1 | 8.4 | 0.133 | 0.129 | 28 |
| Example 16 | Compound 116 | 4.3 | 8.0 | 0.135 | 0.128 | 26 |

As can be seen from the results in Table 1, the organic light emitting diodes of Examples 1-16 using the inventive organic compounds as materials for the hole auxiliary layers showed higher efficiencies and much longer lifetimes than the organic light emitting diode of Comparative Example 1. These results conclude that the inventive organic compounds can be used to fabricate organic light emitting diodes with markedly improved luminescent properties, including high luminous efficiency and long lifetime.

What is claimed is:

1. An organic light emitting compound represented by Formula A: 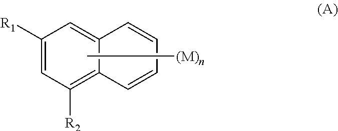
wherein R₁ and R₂ are identical to or different from each other and are each independently Structure A: 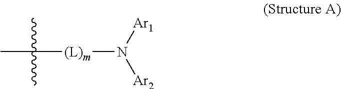
wherein L represents a linker and is a single bond or is selected from substituted or unsubstituted C₁-C₁₀ alkylene groups, substituted or unsubstituted C₂-C₁₀ alkenylene groups, substituted or unsubstituted C₂-C₁₀ alkynylene groups, substituted or unsubstituted C₃-C₂₀ cycloalkylene groups, substituted or unsubstituted C₂-C₃₀ heterocycloalkylene groups, and substituted or unsubstituted C₂-C₃₀ heteroarylene groups, m is an integer from 0 to 4, and Ar₁ and Ar₂ are identical to or different from each other and are each independently a substituted or unsubstituted C₆-C₅₀ aromatic hydrocarbon ring; or Structure B: 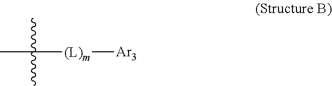
wherein L and m are as defined in Structure A and Ar₃ is a substituted or unsubstituted C₆-C₅₀ aromatic hydrocarbon ring, with the proviso that at least one of R₁ and R₂ is Structure A,
M is hydrogen, deuterium, a substituted or unsubstituted C₁-C₁₀ alkyl group, a substituted or unsubstituted C₂-C₁₀ alkenyl group, a substituted or unsubstituted C₂-C₁₀ alkynyl group, a substituted or unsubstituted C₃-C₂₀ cycloalkyl group, a substituted or unsubstituted C₅-C₂₀ cycloalkenyl group, a substituted or unsubstituted C₁-C₁₀ alkoxy group, a substituted or unsubstituted C₆-C₃₀ aryloxy group, a substituted or unsubstituted C₁-C₃₀ alkylthioxy group, a substituted or unsubstituted C₅-C₃₀ arylthioxy group, a substituted or unsubstituted C₁-C₃₀ alkylamine group, a substituted or unsubstituted C₅-C₃₀ arylamine group, a substituted or unsubstituted C₆-C₃₀ aryl group, a substituted or unsubstituted C₂-C₃₀ heteroaryl group interrupted by one or more heteroatoms selected from O, N, and S, a cyano group, a nitro group, a halogen group, a substituted or unsubstituted silyl group, a substituted or unsubstituted germanium group, a substituted or unsubstituted boron group, a substituted or unsubstituted aluminum group, a carbonyl group, a phosphoryl group, an amino group, a thiol group, a hydroxyl group, a selenium group, a tellurium group, an amide group, an ether group or an ester group, and
n is an integer from 0 to 4, provided that when n is equal to or greater than 2, the plurality of M groups are identical to or different from each other and are each independently optionally combined with an adjacent substituent to form a mono- or polycyclic alicyclic or aromatic ring optionally interrupted by one or more heteroatoms selected from N, S, and O.

2. The organic light emitting compound according to claim 1, wherein either of R₁ and R₂ is Structure A and the other is Structure B.

3. The organic light emitting compound according to claim 1, wherein the compound represented by Formula A is selected from the group consisting of the following compounds:

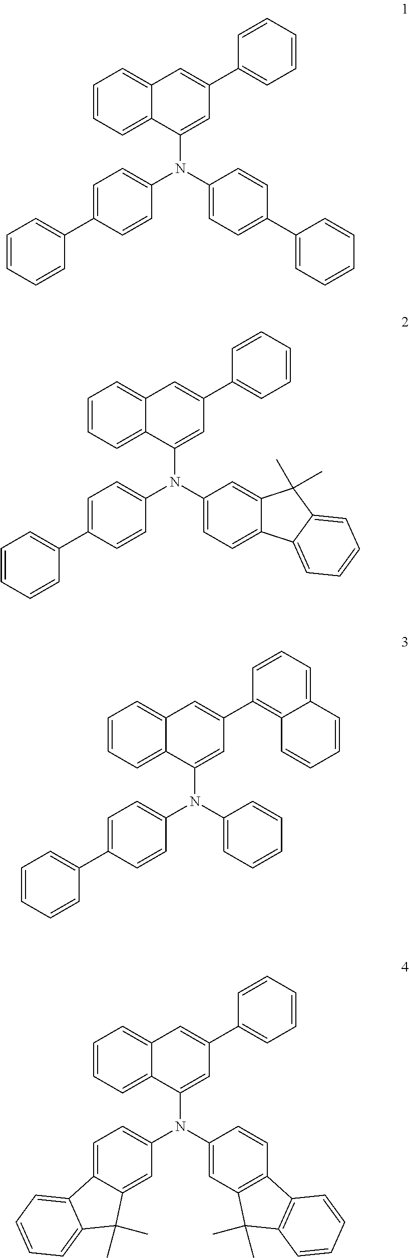 [SMILES: c1ccc(-c2ccc(N(c3ccc(-c4ccccc4)cc3)c3cc(-c4ccccc4)cc4ccccc34)cc2)cc1 ; CC1(C)c2ccccc2-c2ccc(N(c3ccc(-c4ccccc4)cc3)c3cc(-c4ccccc4)cc4ccccc34)cc21 ; c1ccc(-c2ccc(N(c3ccccc3)c3cc(-c4cccc5ccccc45)cc4ccccc34)cc2)cc1 ; CC1(C)c2ccccc2-c2ccc(N(c3ccc4c(c3)C(C)(C)c3ccccc3-4)c3cc(-c4ccccc4)cc4ccccc34)cc21]

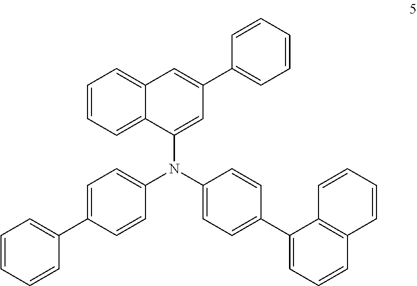 [SMILES: c1ccc(-c2ccc(N(c3ccc(-c4cccc5ccccc45)cc3)c3cc(-c4ccccc4)cc4ccccc34)cc2)cc1]

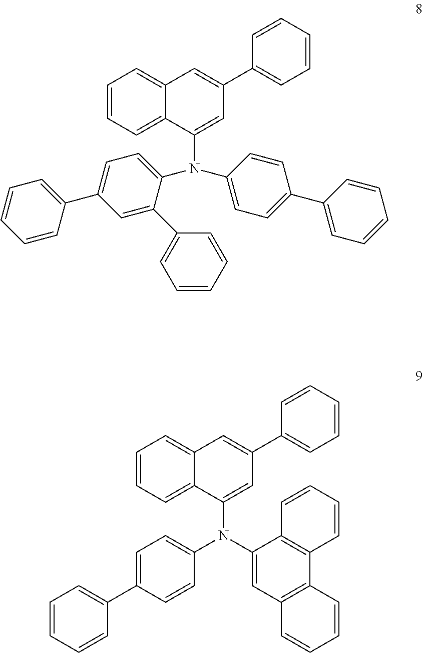 [SMILES: c1ccc(-c2ccc(N(c3ccc(-c4ccccc4)cc3-c3ccccc3)c3cc(-c4ccccc4)cc4ccccc34)cc2)cc1 ; c1ccc(-c2ccc(N(c3cc(-c4ccccc4)cc4ccccc34)c3cc4ccccc4c4ccccc34)cc2)cc1]

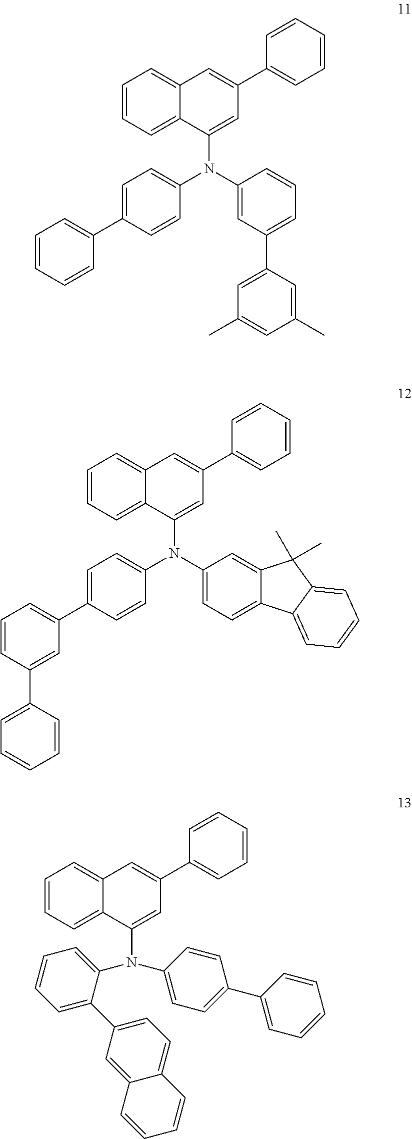 [SMILES: Cc1cc(C)cc(-c2cccc(N(c3ccc(-c4ccccc4)cc3)c3cc(-c4ccccc4)cc4ccccc34)c2)c1 ; CC1(C)c2ccccc2-c2ccc(N(c3ccc(-c4cccc(-c5ccccc5)c4)cc3)c3cc(-c4ccccc4)cc4ccccc34)cc21 ; c1ccc(-c2ccc(N(c3ccccc3-c3ccc4ccccc4c3)c3cc(-c4ccccc4)cc4ccccc34)cc2)cc1]

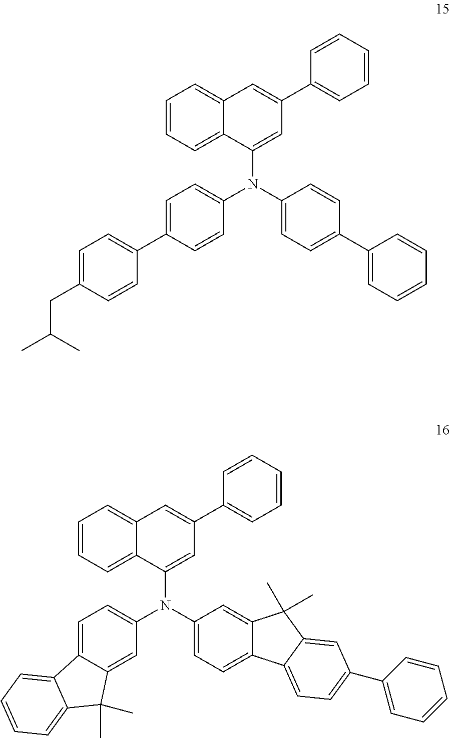 [SMILES: CC(C)Cc1ccc(-c2ccc(N(c3ccc(-c4ccccc4)cc3)c3cc(-c4ccccc4)cc4ccccc34)cc2)cc1 ; CC1(C)c2ccccc2-c2ccc(N(c3ccc4c(c3)C(C)(C)c3cc(-c5ccccc5)ccc3-4)c3cc(-c4ccccc4)cc4ccccc34)cc21]

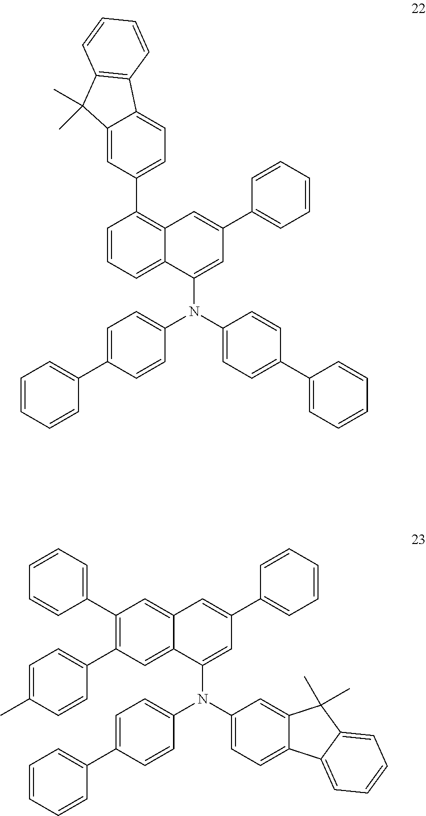 [SMILES: CC1(C)c2ccccc2-c2ccc(-c3cccc4c(N(c5ccc(-c6ccccc6)cc5)c5ccc(-c6ccccc6)cc5)cc(-c5ccccc5)cc34)cc21 ; Cc1ccc(-c2cc3c(N(c4ccc(-c5ccccc5)cc4)c4ccc5c(c4)C(C)(C)c4ccccc4-5)cc(-c4ccccc4)cc3cc2-c2ccccc2)cc1]

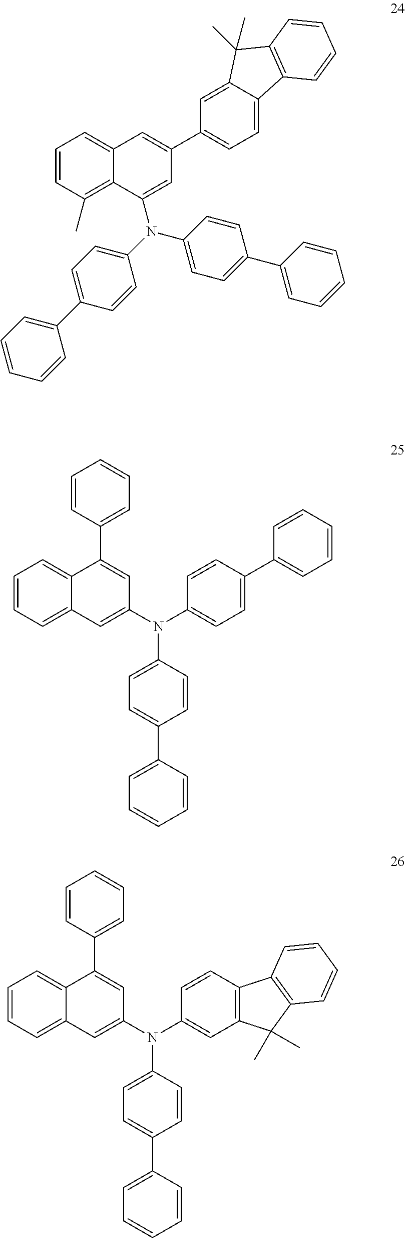 [SMILES: Cc1cccc2cc(-c3ccc4c(c3)C(C)(C)c3ccccc3-4)cc(N(c3ccc(-c4ccccc4)cc3)c3ccc(-c4ccccc4)cc3)c12 ; c1ccc(-c2ccc(N(c3ccc(-c4ccccc4)cc3)c3cc(-c4ccccc4)c4ccccc4c3)cc2)cc1 ; CC1(C)c2ccccc2-c2ccc(N(c3ccc(-c4ccccc4)cc3)c3cc(-c4ccccc4)c4ccccc4c3)cc21]

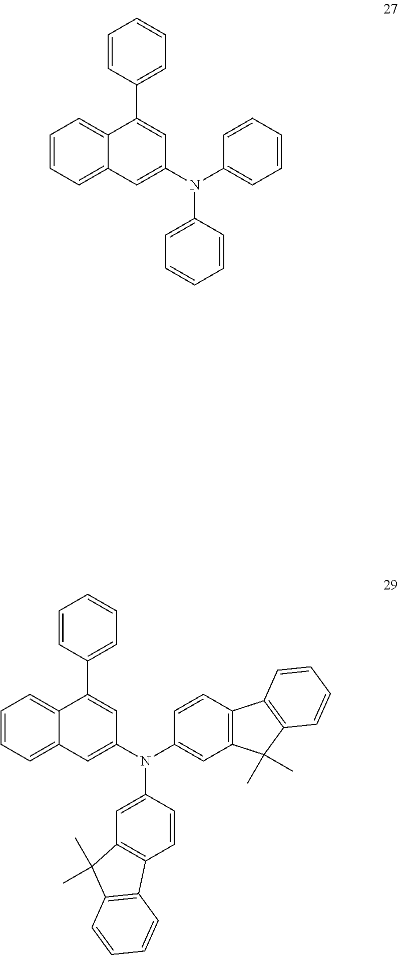 [SMILES: c1ccc(-c2cc(N(c3ccccc3)c3ccccc3)cc3ccccc23)cc1 ; CC1(C)c2ccccc2-c2ccc(N(c3ccc4c(c3)C(C)(C)c3ccccc3-4)c3cc(-c4ccccc4)c4ccccc4c3)cc21]

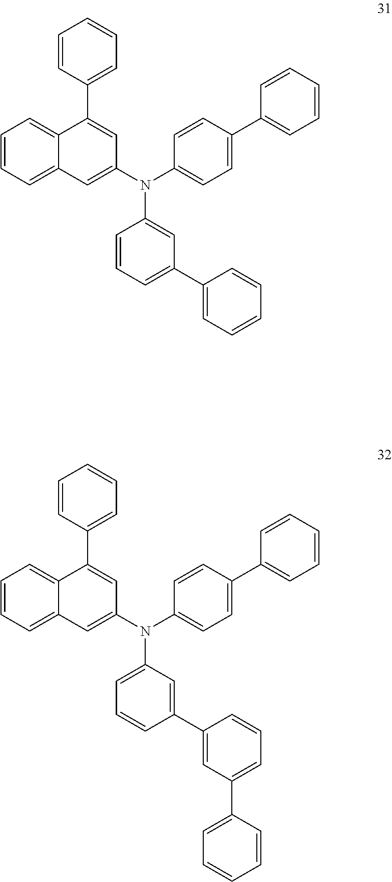 [SMILES: c1ccc(-c2ccc(N(c3cccc(-c4ccccc4)c3)c3cc(-c4ccccc4)c4ccccc4c3)cc2)cc1 ; c1ccc(-c2ccc(N(c3cccc(-c4cccc(-c5ccccc5)c4)c3)c3cc(-c4ccccc4)c4ccccc4c3)cc2)cc1]

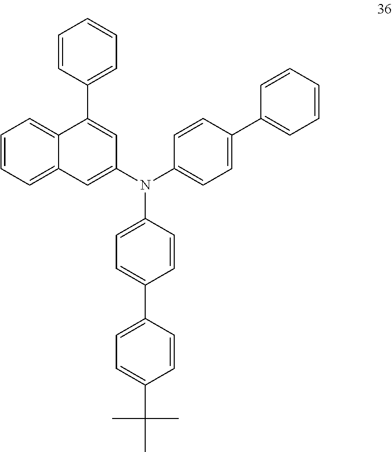 [SMILES: CC(C)(C)c1ccc(-c2ccc(N(c3ccc(-c4ccccc4)cc3)c3cc(-c4ccccc4)c4ccccc4c3)cc2)cc1]

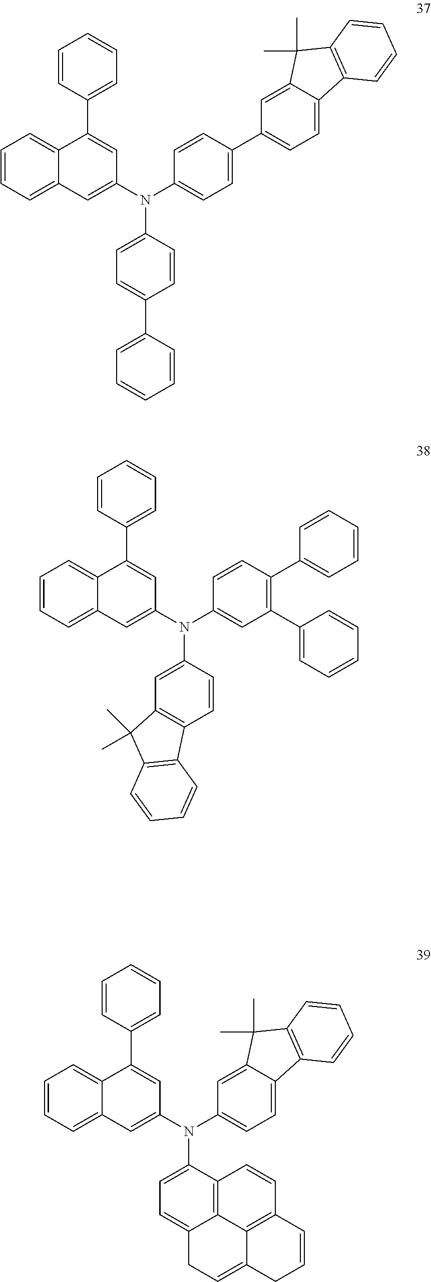 [SMILES: CC1(C)c2ccccc2-c2ccc(-c3ccc(N(c4ccc(-c5ccccc5)cc4)c4cc(-c5ccccc5)c5ccccc5c4)cc3)cc21 ; CC1(C)c2ccccc2-c2ccc(N(c3ccc(-c4ccccc4)c(-c4ccccc4)c3)c3cc(-c4ccccc4)c4ccccc4c3)cc21 ; CC1(C)c2ccccc2-c2ccc(N(c3cc(-c4ccccc4)c4ccccc4c3)c3ccc4c5c6c(ccc35)C=CCC6=CC4)cc21]

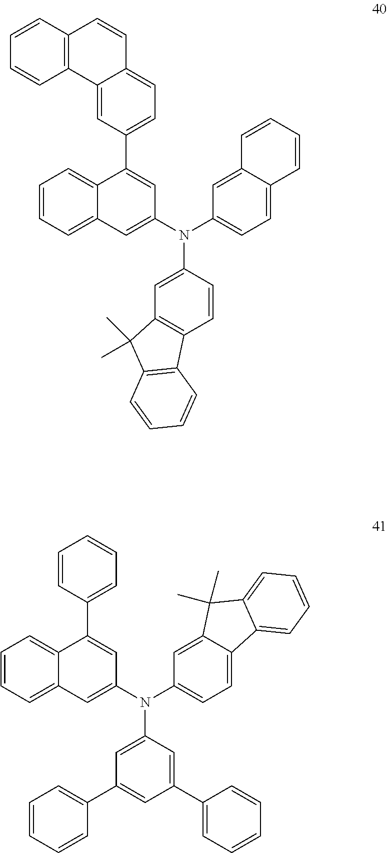 [SMILES: CC1(C)c2ccccc2-c2ccc(N(c3ccc4ccccc4c3)c3cc(-c4ccc5ccc6ccccc6c5c4)c4ccccc4c3)cc21 ; CC1(C)c2ccccc2-c2ccc(N(c3cc(-c4ccccc4)cc(-c4ccccc4)c3)c3cc(-c4ccccc4)c4ccccc4c3)cc21]

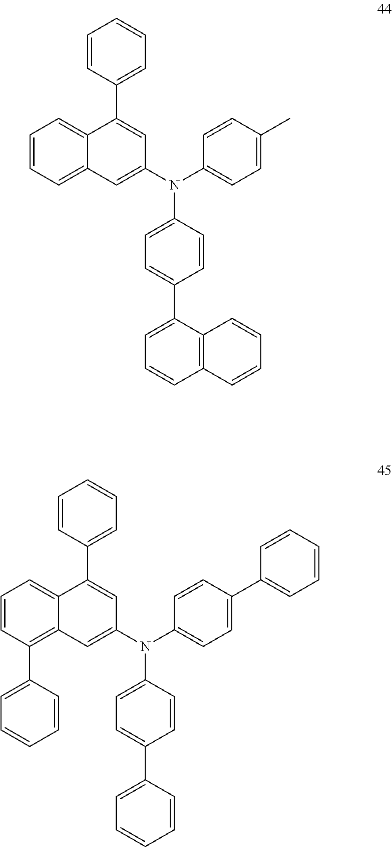 [SMILES: Cc1ccc(N(c2ccc(-c3cccc4ccccc34)cc2)c2cc(-c3ccccc3)c3ccccc3c2)cc1 ; c1ccc(-c2ccc(N(c3ccc(-c4ccccc4)cc3)c3cc(-c4ccccc4)c4cccc(-c5ccccc5)c4c3)cc2)cc1]

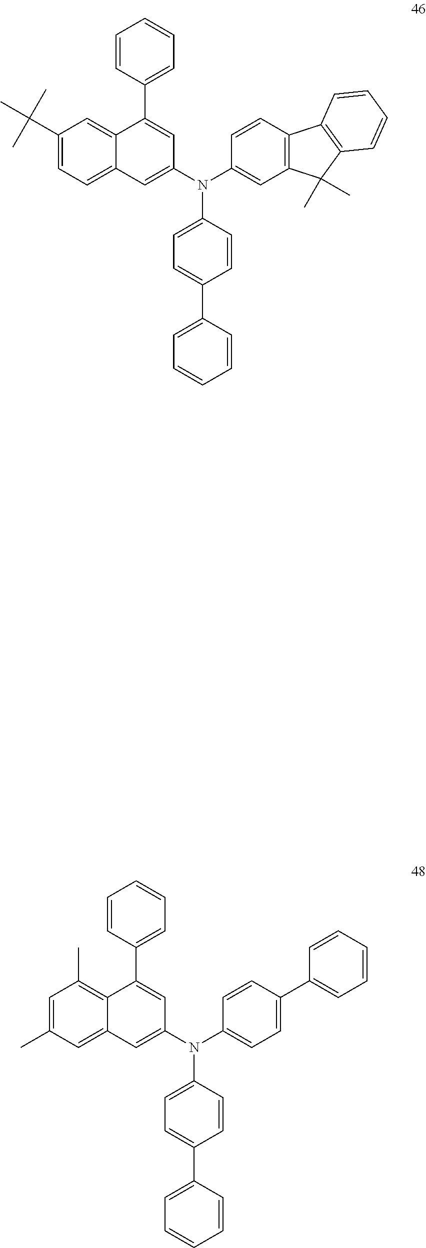 [SMILES: CC(C)(C)c1ccc2cc(N(c3ccc(-c4ccccc4)cc3)c3ccc4c(c3)C(C)(C)c3ccccc3-4)cc(-c3ccccc3)c2c1 ; Cc1cc(C)c2c(-c3ccccc3)cc(N(c3ccc(-c4ccccc4)cc3)c3ccc(-c4ccccc4)cc3)cc2c1]

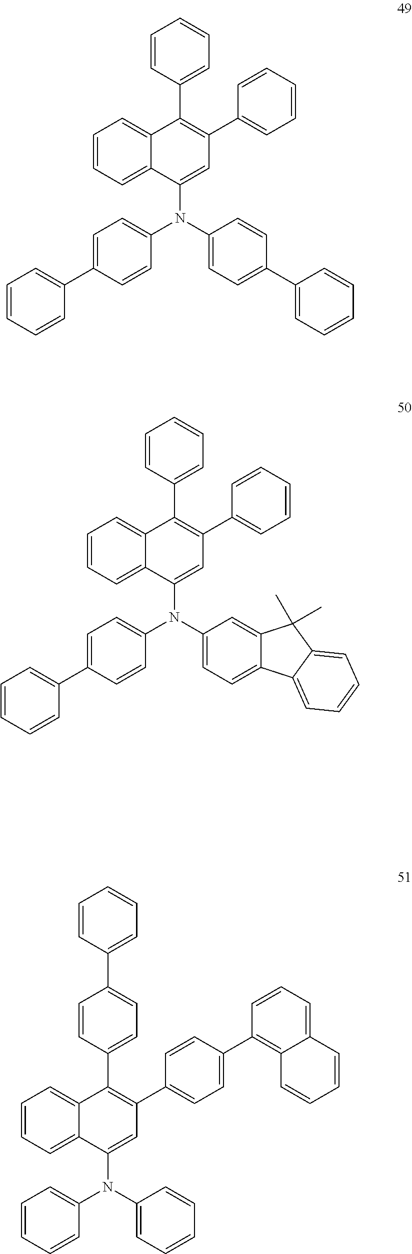 [SMILES: c1ccc(-c2ccc(N(c3ccc(-c4ccccc4)cc3)c3cc(-c4ccccc4)c(-c4ccccc4)c4ccccc34)cc2)cc1 ; CC1(C)c2ccccc2-c2ccc(N(c3ccc(-c4ccccc4)cc3)c3cc(-c4ccccc4)c(-c4ccccc4)c4ccccc34)cc21 ; c1ccc(-c2ccc(-c3c(-c4ccc(-c5cccc6ccccc56)cc4)cc(N(c4ccccc4)c4ccccc4)c4ccccc34)cc2)cc1]

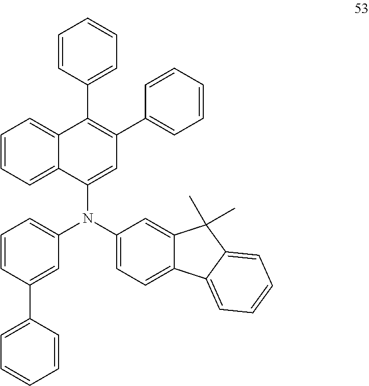 [SMILES: CC1(C)c2ccccc2-c2ccc(N(c3cccc(-c4ccccc4)c3)c3cc(-c4ccccc4)c(-c4ccccc4)c4ccccc34)cc21]

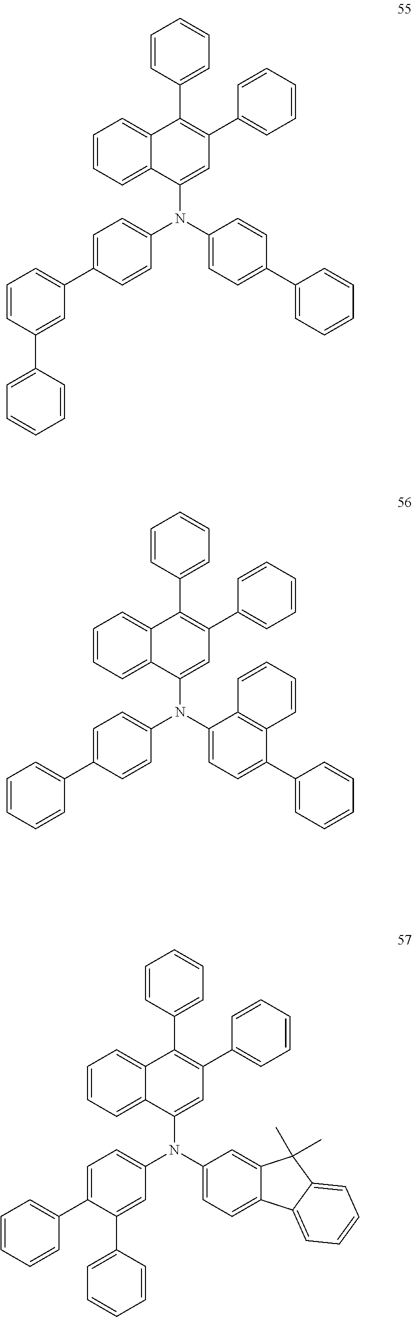 [SMILES: c1ccc(-c2ccc(N(c3ccc(-c4cccc(-c5ccccc5)c4)cc3)c3cc(-c4ccccc4)c(-c4ccccc4)c4ccccc34)cc2)cc1 ; c1ccc(-c2ccc(N(c3ccc(-c4ccccc4)c4ccccc34)c3cc(-c4ccccc4)c(-c4ccccc4)c4ccccc34)cc2)cc1 ; CC1(C)c2ccccc2-c2ccc(N(c3ccc(-c4ccccc4)c(-c4ccccc4)c3)c3cc(-c4ccccc4)c(-c4ccccc4)c4ccccc34)cc21]

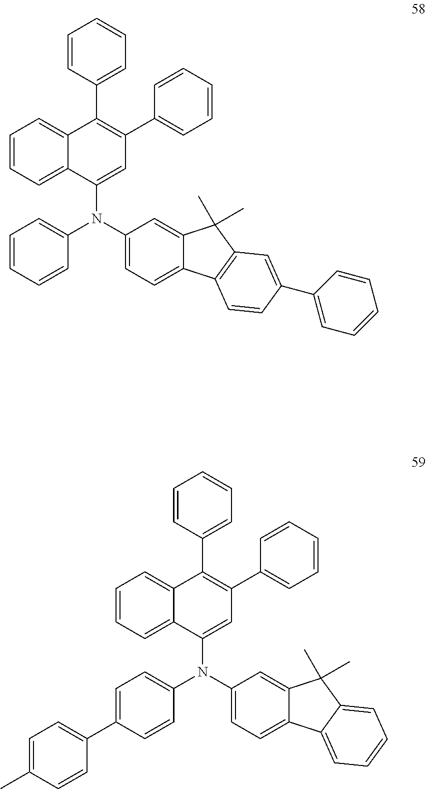 [SMILES: CC1(C)c2cc(-c3ccccc3)ccc2-c2ccc(N(c3ccccc3)c3cc(-c4ccccc4)c(-c4ccccc4)c4ccccc34)cc21 ; Cc1ccc(-c2ccc(N(c3ccc4c(c3)C(C)(C)c3ccccc3-4)c3cc(-c4ccccc4)c(-c4ccccc4)c4ccccc34)cc2)cc1]

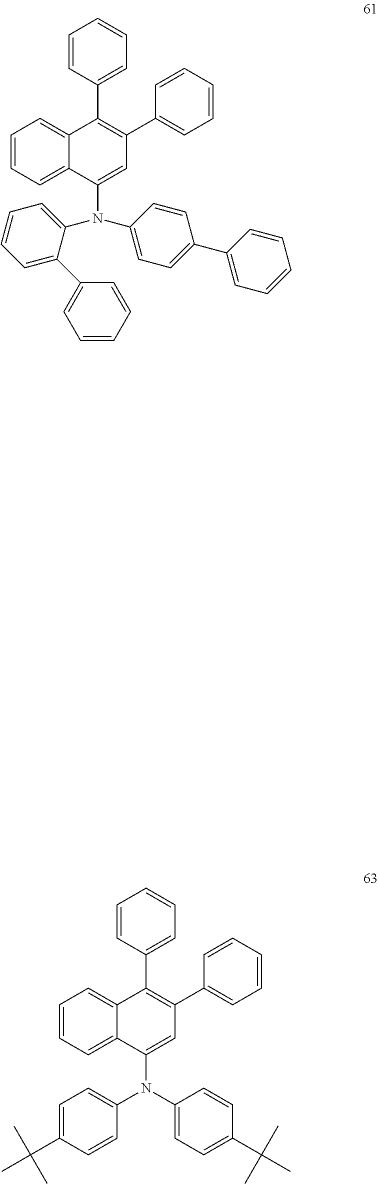 [SMILES: c1ccc(-c2ccc(N(c3ccccc3-c3ccccc3)c3cc(-c4ccccc4)c(-c4ccccc4)c4ccccc34)cc2)cc1 ; CC(C)(C)c1ccc(N(c2ccc(C(C)(C)C)cc2)c2cc(-c3ccccc3)c(-c3ccccc3)c3ccccc23)cc1]

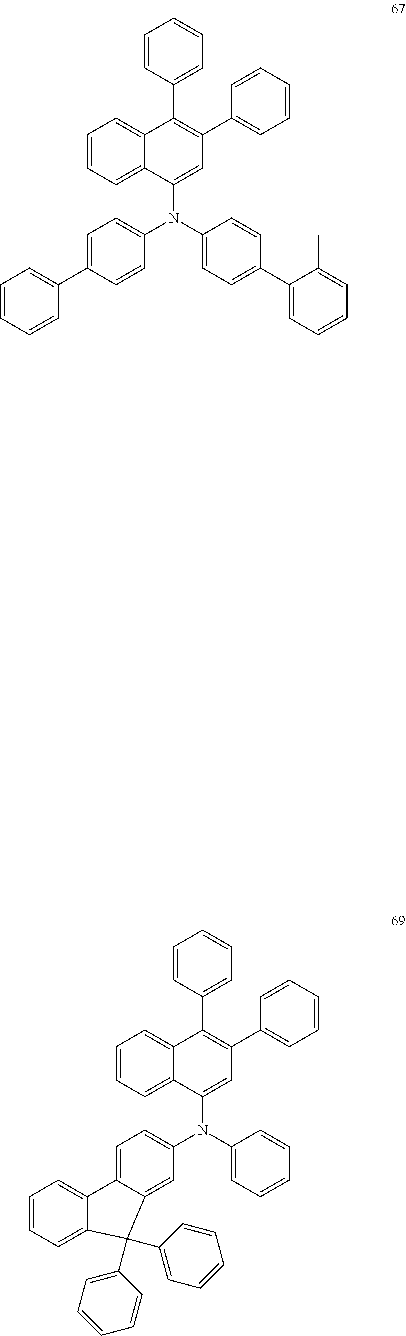 [SMILES: Cc1ccccc1-c1ccc(N(c2ccc(-c3ccccc3)cc2)c2cc(-c3ccccc3)c(-c3ccccc3)c3ccccc23)cc1 ; c1ccc(-c2cc(N(c3ccccc3)c3ccc4c(c3)C(c3ccccc3)(c3ccccc3)c3ccccc3-4)c3ccccc3c2-c2ccccc2)cc1]

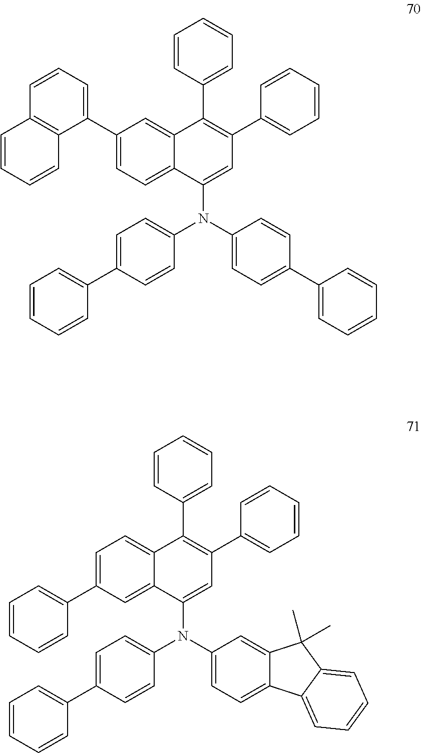 [SMILES: c1ccc(-c2ccc(N(c3ccc(-c4ccccc4)cc3)c3cc(-c4ccccc4)c(-c4ccccc4)c4cc(-c5cccc6ccccc56)ccc34)cc2)cc1 ; CC1(C)c2ccccc2-c2ccc(N(c3ccc(-c4ccccc4)cc3)c3cc(-c4ccccc4)c(-c4ccccc4)c4ccc(-c5ccccc5)cc34)cc21]

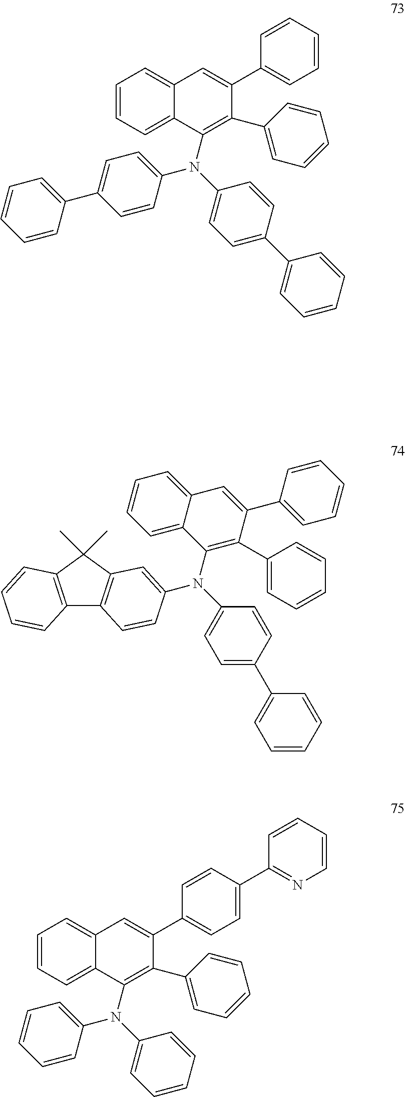 [SMILES: c1ccc(-c2ccc(N(c3ccc(-c4ccccc4)cc3)c3c(-c4ccccc4)c(-c4ccccc4)cc4ccccc34)cc2)cc1 ; CC1(C)c2ccccc2-c2ccc(N(c3ccc(-c4ccccc4)cc3)c3c(-c4ccccc4)c(-c4ccccc4)cc4ccccc34)cc21 ; c1ccc(-c2c(-c3ccc(-c4ccccn4)cc3)cc3ccccc3c2N(c2ccccc2)c2ccccc2)cc1]

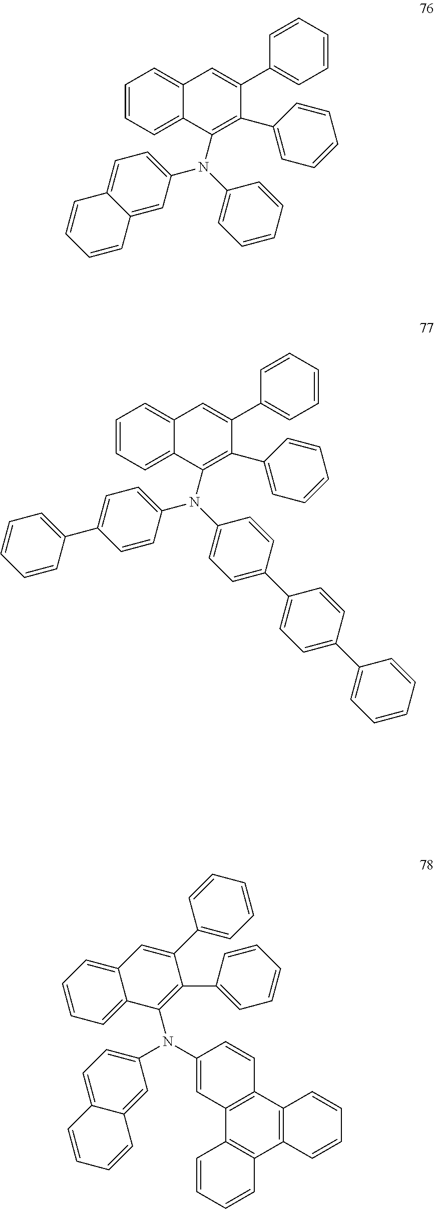 [SMILES: c1ccc(-c2cc3ccccc3c(N(c3ccccc3)c3ccc4ccccc4c3)c2-c2ccccc2)cc1 ; c1ccc(-c2ccc(-c3ccc(N(c4ccc(-c5ccccc5)cc4)c4c(-c5ccccc5)c(-c5ccccc5)cc5ccccc45)cc3)cc2)cc1 ; c1ccc(-c2cc3ccccc3c(N(c3ccc4ccccc4c3)c3ccc4c5ccccc5c5ccccc5c4c3)c2-c2ccccc2)cc1]

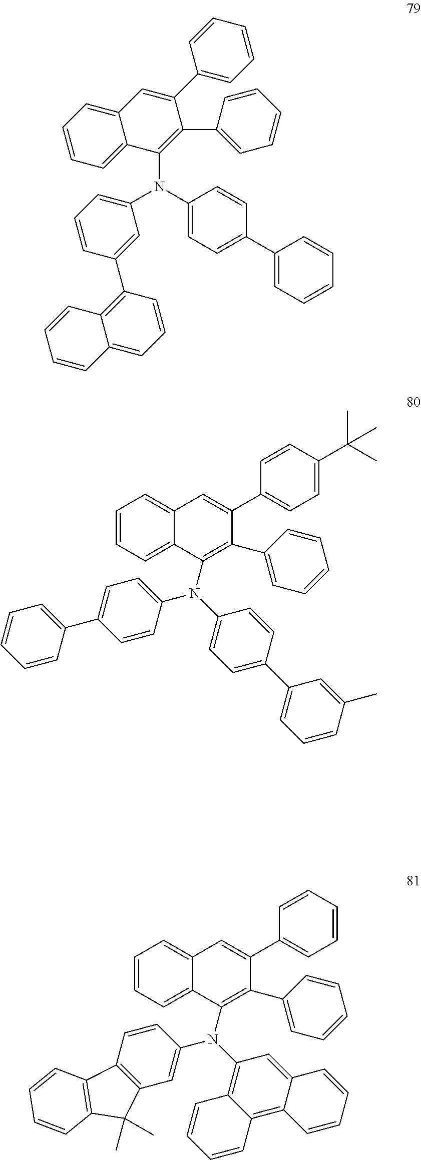 [SMILES: c1ccc(-c2ccc(N(c3cccc(-c4cccc5ccccc45)c3)c3c(-c4ccccc4)c(-c4ccccc4)cc4ccccc34)cc2)cc1 ; Cc1cccc(-c2ccc(N(c3ccc(-c4ccccc4)cc3)c3c(-c4ccccc4)c(-c4ccc(C(C)(C)C)cc4)cc4ccccc34)cc2)c1 ; CC1(C)c2ccccc2-c2ccc(N(c3c(-c4ccccc4)c(-c4ccccc4)cc4ccccc34)c3cc4ccccc4c4ccccc34)cc21]

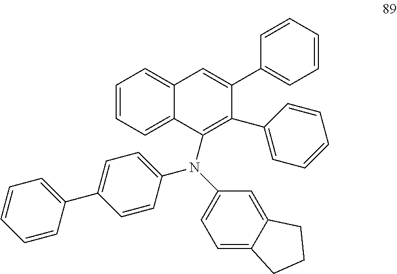 [SMILES: c1ccc(-c2ccc(N(c3ccc4c(c3)CCC4)c3c(-c4ccccc4)c(-c4ccccc4)cc4ccccc34)cc2)cc1]

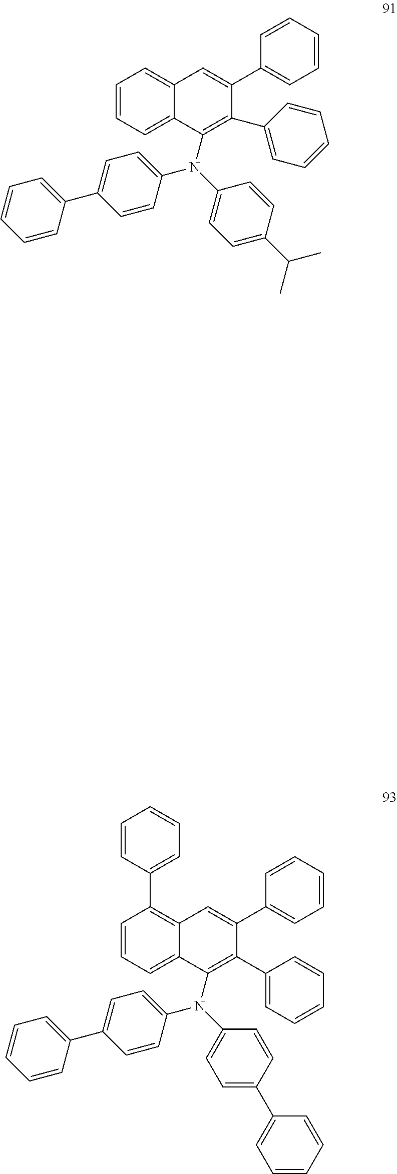 [SMILES: CC(C)c1ccc(N(c2ccc(-c3ccccc3)cc2)c2c(-c3ccccc3)c(-c3ccccc3)cc3ccccc23)cc1 ; c1ccc(-c2ccc(N(c3ccc(-c4ccccc4)cc3)c3c(-c4ccccc4)c(-c4ccccc4)cc4c(-c5ccccc5)cccc34)cc2)cc1]

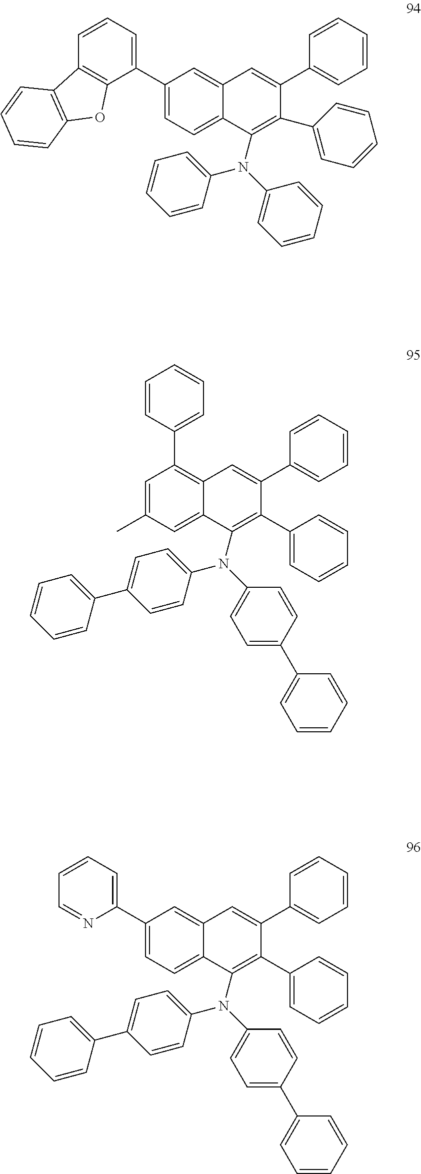 [SMILES: c1ccc(-c2cc3cc(-c4cccc5c4oc4ccccc45)ccc3c(N(c3ccccc3)c3ccccc3)c2-c2ccccc2)cc1 ; Cc1cc(-c2ccccc2)c2cc(-c3ccccc3)c(-c3ccccc3)c(N(c3ccc(-c4ccccc4)cc3)c3ccc(-c4ccccc4)cc3)c2c1 ; c1ccc(-c2ccc(N(c3ccc(-c4ccccc4)cc3)c3c(-c4ccccc4)c(-c4ccccc4)cc4cc(-c5ccccn5)ccc34)cc2)cc1]

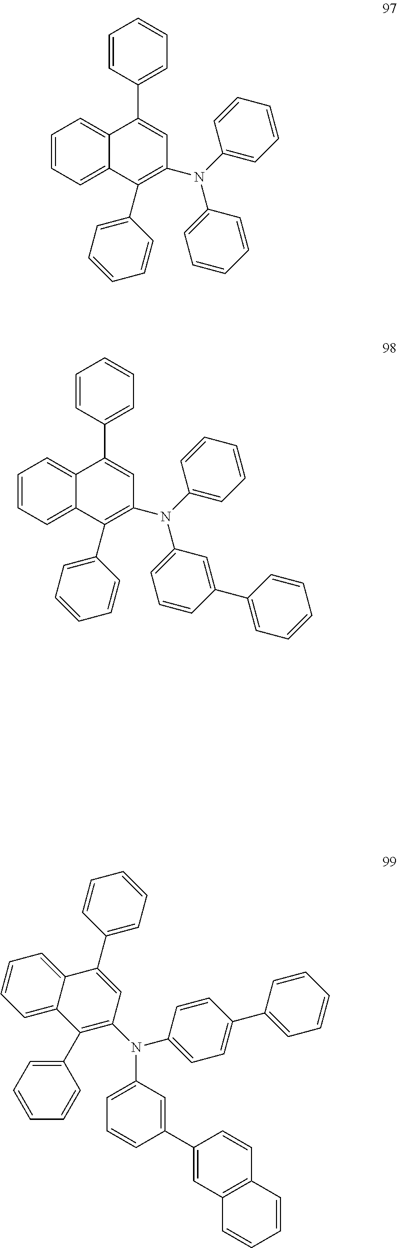 [SMILES: c1ccc(-c2cc(N(c3ccccc3)c3ccccc3)c(-c3ccccc3)c3ccccc23)cc1 ; c1ccc(-c2cccc(N(c3ccccc3)c3cc(-c4ccccc4)c4ccccc4c3-c3ccccc3)c2)cc1 ; c1ccc(-c2ccc(N(c3cccc(-c4ccc5ccccc5c4)c3)c3cc(-c4ccccc4)c4ccccc4c3-c3ccccc3)cc2)cc1]

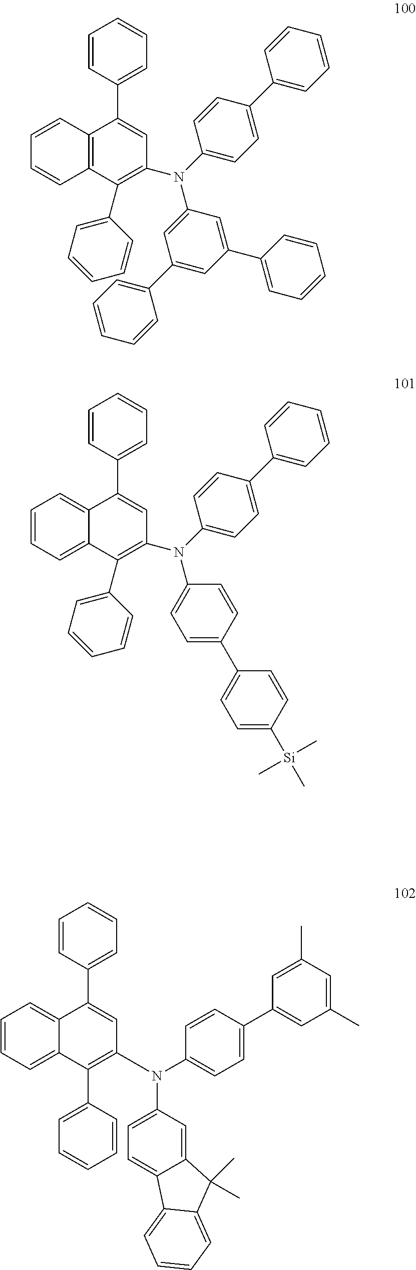 [SMILES: c1ccc(-c2ccc(N(c3cc(-c4ccccc4)cc(-c4ccccc4)c3)c3cc(-c4ccccc4)c4ccccc4c3-c3ccccc3)cc2)cc1 ; C[Si](C)(C)c1ccc(-c2ccc(N(c3ccc(-c4ccccc4)cc3)c3cc(-c4ccccc4)c4ccccc4c3-c3ccccc3)cc2)cc1 ; Cc1cc(C)cc(-c2ccc(N(c3ccc4c(c3)C(C)(C)c3ccccc3-4)c3cc(-c4ccccc4)c4ccccc4c3-c3ccccc3)cc2)c1]

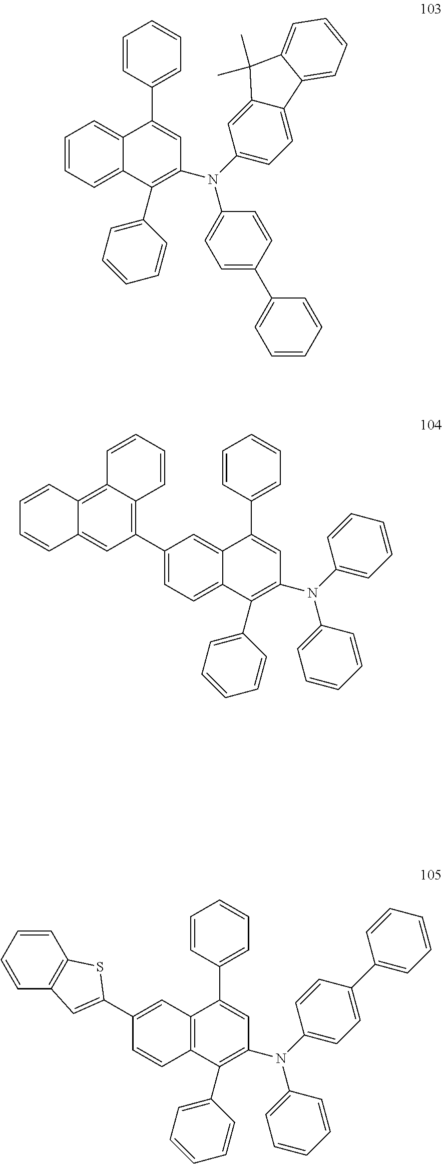 [SMILES: CC1(C)c2ccccc2-c2ccc(N(c3ccc(-c4ccccc4)cc3)c3cc(-c4ccccc4)c4ccccc4c3-c3ccccc3)cc21 ; c1ccc(-c2cc(N(c3ccccc3)c3ccccc3)c(-c3ccccc3)c3ccc(-c4cc5ccccc5c5ccccc45)cc23)cc1 ; c1ccc(-c2ccc(N(c3ccccc3)c3cc(-c4ccccc4)c4cc(-c5cc6ccccc6s5)ccc4c3-c3ccccc3)cc2)cc1]

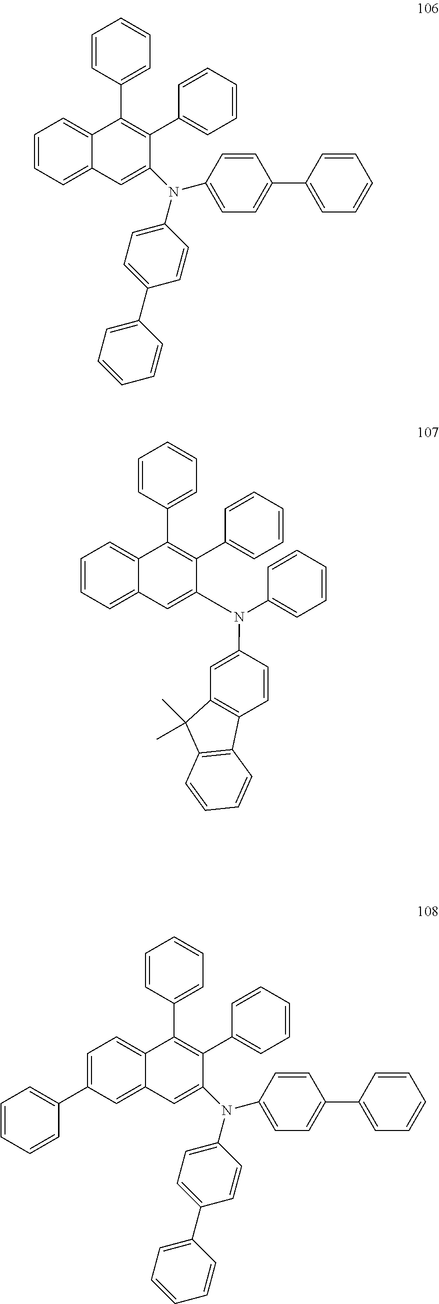 [SMILES: c1ccc(-c2ccc(N(c3ccc(-c4ccccc4)cc3)c3cc4ccccc4c(-c4ccccc4)c3-c3ccccc3)cc2)cc1 ; CC1(C)c2ccccc2-c2ccc(N(c3ccccc3)c3cc4ccccc4c(-c4ccccc4)c3-c3ccccc3)cc21 ; c1ccc(-c2ccc(N(c3ccc(-c4ccccc4)cc3)c3cc4cc(-c5ccccc5)ccc4c(-c4ccccc4)c3-c3ccccc3)cc2)cc1]

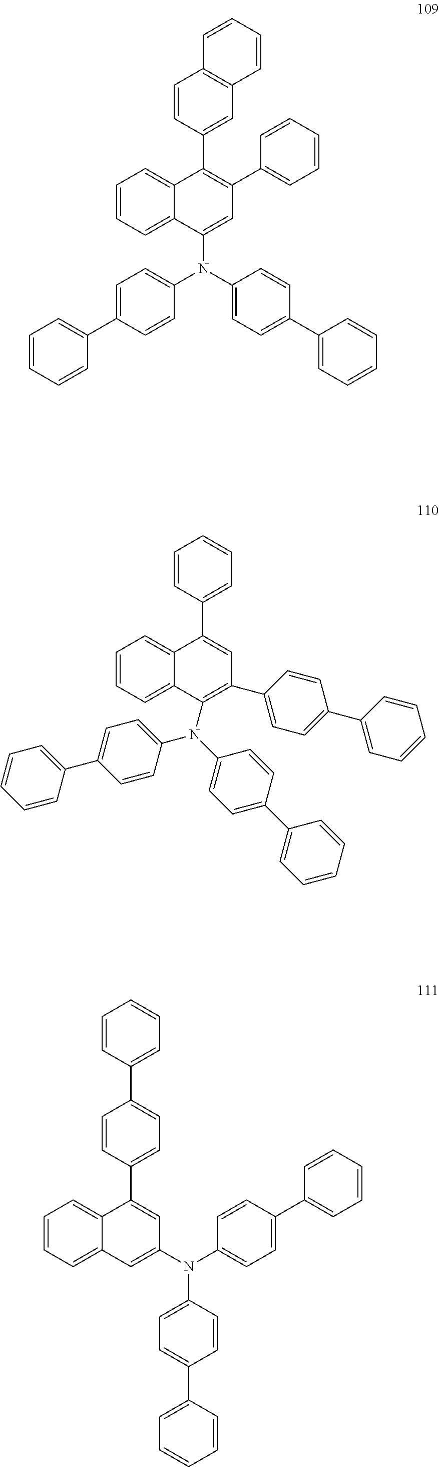 [SMILES: c1ccc(-c2ccc(N(c3ccc(-c4ccccc4)cc3)c3cc(-c4ccccc4)c(-c4ccc5ccccc5c4)c4ccccc34)cc2)cc1 ; c1ccc(-c2ccc(-c3cc(-c4ccccc4)c4ccccc4c3N(c3ccc(-c4ccccc4)cc3)c3ccc(-c4ccccc4)cc3)cc2)cc1 ; c1ccc(-c2ccc(-c3cc(N(c4ccc(-c5ccccc5)cc4)c4ccc(-c5ccccc5)cc4)cc4ccccc34)cc2)cc1]

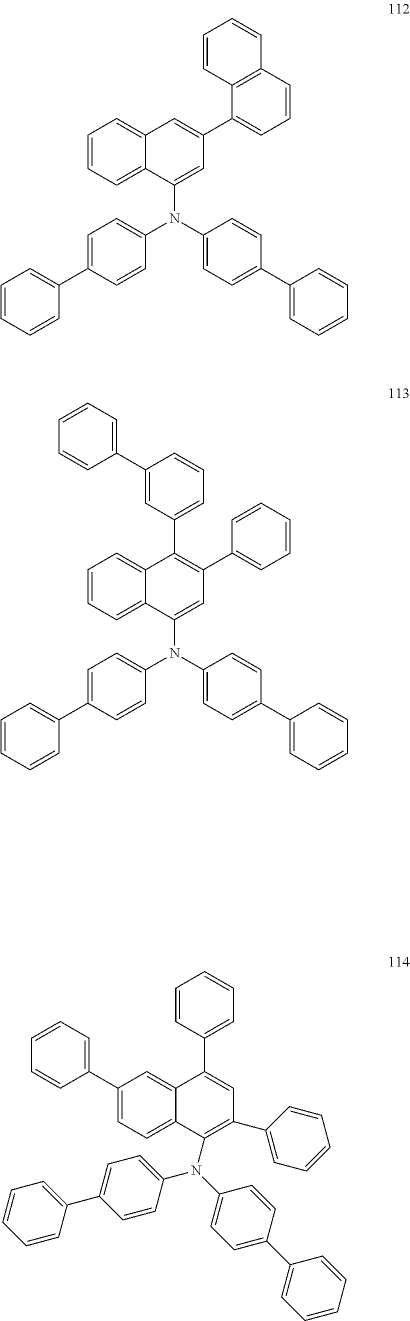 [SMILES: c1ccc(-c2ccc(N(c3ccc(-c4ccccc4)cc3)c3cc(-c4cccc5ccccc45)cc4ccccc34)cc2)cc1 ; c1ccc(-c2ccc(N(c3ccc(-c4ccccc4)cc3)c3cc(-c4ccccc4)c(-c4cccc(-c5ccccc5)c4)c4ccccc34)cc2)cc1 ; c1ccc(-c2ccc(N(c3ccc(-c4ccccc4)cc3)c3c(-c4ccccc4)cc(-c4ccccc4)c4cc(-c5ccccc5)ccc34)cc2)cc1]

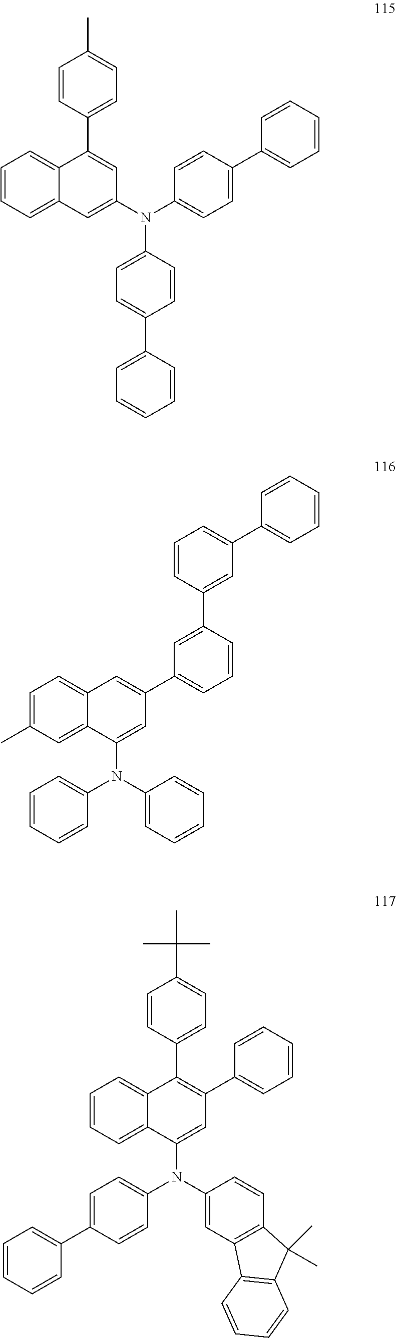 [SMILES: Cc1ccc(-c2cc(N(c3ccc(-c4ccccc4)cc3)c3ccc(-c4ccccc4)cc3)cc3ccccc23)cc1 ; Cc1ccc2cc(-c3cccc(-c4cccc(-c5ccccc5)c4)c3)cc(N(c3ccccc3)c3ccccc3)c2c1 ; CC(C)(C)c1ccc(-c2c(-c3ccccc3)cc(N(c3ccc(-c4ccccc4)cc3)c3ccc4c(c3)-c3ccccc3C4(C)C)c3ccccc23)cc1]

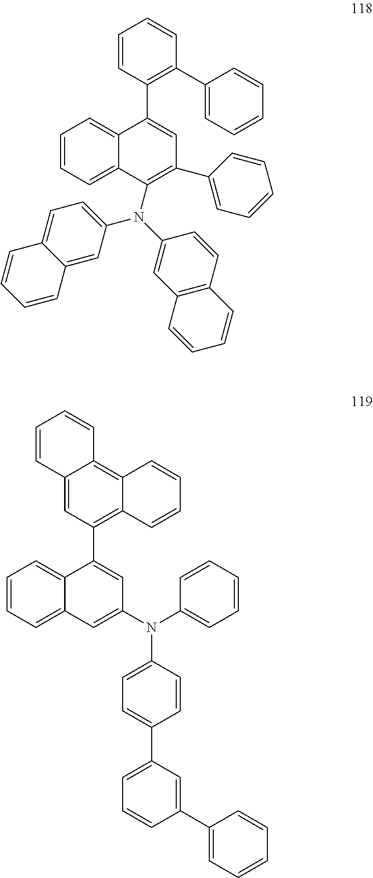 [SMILES: c1ccc(-c2ccccc2-c2cc(-c3ccccc3)c(N(c3ccc4ccccc4c3)c3ccc4ccccc4c3)c3ccccc23)cc1 ; c1ccc(-c2cccc(-c3ccc(N(c4ccccc4)c4cc(-c5cc6ccccc6c6ccccc56)c5ccccc5c4)cc3)c2)cc1]

4. An organic light emitting diode comprising a first electrode, a second electrode opposite to the first electrode, and one or more organic layers interposed between the first and second electrodes wherein one of the organic layers comprises the organic light emitting compound according to claim 1 and optionally another organic light emitting compound according to claim 1.

5. The organic light emitting diode according to claim 4, wherein the organic layers are selected from a hole injecting layer, a hole transport layer, a hole auxiliary layer, a light emitting layer, an electron transport layer, and an electron injecting layer.

6. The organic light emitting diode according to claim 4, wherein the organic light emitting compound is present in a hole auxiliary layer interposed between the first and second electrodes.

7. An organic light emitting compound represented by Formula A: 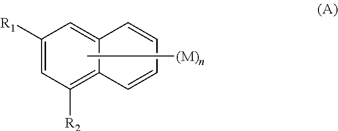
wherein R₁ and R₂ are identical to or different from each other and are each independently Structure A: 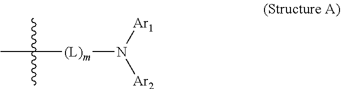
wherein L represents a linker and is selected from substituted or unsubstituted C₁-C₁₀ alkylene groups, substituted or unsubstituted C₂-C₁₀ alkenylene groups, substituted or unsubstituted C₂-C₁₀ alkynylene groups, substituted or unsubstituted C₃-C₂₀ cycloalkylene groups, substituted or unsubstituted C₂-C₃₀ heterocycloalkylene groups, substituted or unsubstituted C₆-C₃₀ arylene groups, and substituted or unsubstituted C₂-C₃₀ heteroarylene groups, m is an integer from 0 to 4, and Ar₁ and Ar₂ are identical to or different from each other and are each independently a substituted or unsubstituted C₂-C₄₀ aromatic heterocyclic group; or Structure B: 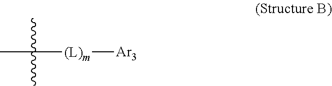
wherein L and m are as defined in Structure A and Ar₃ is a substituted or unsubstituted C₆-C₅₀ aromatic hydrocarbon ring, with the proviso that at least one of R₁ and R₂ is Structure A,
M is hydrogen, deuterium, a substituted or unsubstituted C₁-C₁₀ alkyl group, a substituted or unsubstituted C₂-C₁₀ alkenyl group, a substituted or unsubstituted C₂-C₁₀ alkynyl group, a substituted or unsubstituted C₃-C₂₀ cycloalkyl group, a substituted or unsubstituted C₅-C₂₀ cycloalkenyl group, a substituted or unsubstituted C₁-C₁₀ alkoxy group, a substituted or unsubstituted C₆-C₃₀ aryloxy group, a substituted or unsubstituted C₁-C₃₀ alkylthioxy group, a substituted or unsubstituted C₅-C₃₀ arylthioxy group, a substituted or unsubstituted C₁-C₃₀ alkylamine group, a substituted or unsubstituted C₅-C₃₀ arylamine group, a substituted or unsubstituted C₆-C₃₀ aryl group, a substituted or unsubstituted C₂-C₃₀ heteroaryl group interrupted by one or more heteroatoms selected from O, N, and S, a cyano group, a nitro group, a halogen group, a substituted or unsubstituted silyl group, a substituted or unsubstituted germanium group, a substituted or unsubstituted boron group, a substituted or unsubstituted aluminum group, a carbonyl group, a phosphoryl group, an amino group, a thiol group, a hydroxyl group, a selenium group, a tellurium group, an amide group, an ether group or an ester group, and
n is an integer from 0 to 4, provided that when n is equal to or greater than 2, the plurality of M groups are identical to or different from each other and are each independently optionally combined with an adjacent substituent to form a mono- or polycyclic alicyclic or aromatic ring optionally interrupted by one or more heteroatoms selected from N, S, and O.

8. The organic light emitting compound according to claim 7, wherein either of R₁ and R₂ is Structure A and the other is Structure B.

9. An organic light emitting diode comprising a first electrode, a second electrode opposite to the first electrode, and one or more organic layers interposed between the first and second electrodes wherein one of the organic layers comprises the organic light emitting compound according to claim 7 and optionally another organic light emitting compound according to claim 7.

10. The organic light emitting diode according to claim 9, wherein the organic layers are selected from a hole injecting layer, a hole transport layer, a hole auxiliary layer, a light emitting layer, an electron transport layer, and an electron injecting layer.

11. The organic light emitting diode according to claim 9, wherein the organic light emitting compound is present in a hole auxiliary layer interposed between the first and second electrodes.

12. An organic light emitting compound selected from the group consisting of the following compounds:

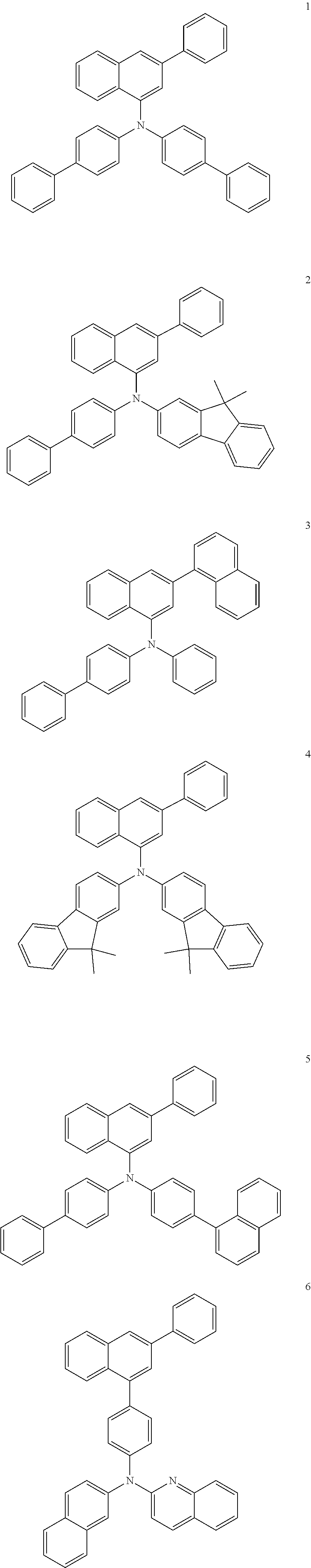 [SMILES: c1ccc(-c2ccc(N(c3ccc(-c4ccccc4)cc3)c3cc(-c4ccccc4)cc4ccccc34)cc2)cc1 ; CC1(C)c2ccccc2-c2ccc(N(c3ccc(-c4ccccc4)cc3)c3cc(-c4ccccc4)cc4ccccc34)cc21 ; c1ccc(-c2ccc(N(c3ccccc3)c3cc(-c4cccc5ccccc45)cc4ccccc34)cc2)cc1 ; CC1(C)c2ccccc2-c2ccc(N(c3ccc4c(c3)C(C)(C)c3ccccc3-4)c3cc(-c4ccccc4)cc4ccccc34)cc21 ; c1ccc(-c2ccc(N(c3ccc(-c4cccc5ccccc45)cc3)c3cc(-c4ccccc4)cc4ccccc34)cc2)cc1 ; c1ccc(-c2cc(-c3ccc(N(c4ccc5ccccc5c4)c4ccc5ccccc5n4)cc3)c3ccccc3c2)cc1]

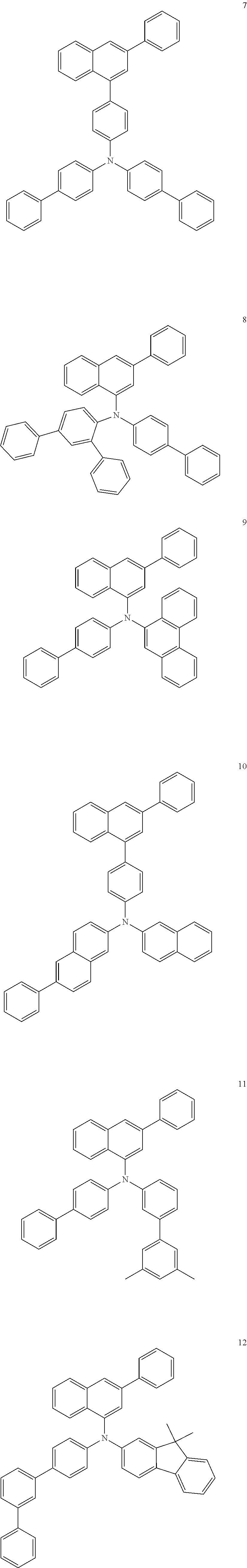 [SMILES: c1ccc(-c2ccc(N(c3ccc(-c4ccccc4)cc3)c3ccc(-c4cc(-c5ccccc5)cc5ccccc45)cc3)cc2)cc1 ; c1ccc(-c2ccc(N(c3ccc(-c4ccccc4)cc3-c3ccccc3)c3cc(-c4ccccc4)cc4ccccc34)cc2)cc1 ; c1ccc(-c2ccc(N(c3cc(-c4ccccc4)cc4ccccc34)c3cc4ccccc4c4ccccc34)cc2)cc1 ; c1ccc(-c2ccc3cc(N(c4ccc(-c5cc(-c6ccccc6)cc6ccccc56)cc4)c4ccc5ccccc5c4)ccc3c2)cc1 ; Cc1cc(C)cc(-c2cccc(N(c3ccc(-c4ccccc4)cc3)c3cc(-c4ccccc4)cc4ccccc34)c2)c1 ; CC1(C)c2ccccc2-c2ccc(N(c3ccc(-c4cccc(-c5ccccc5)c4)cc3)c3cc(-c4ccccc4)cc4ccccc34)cc21]

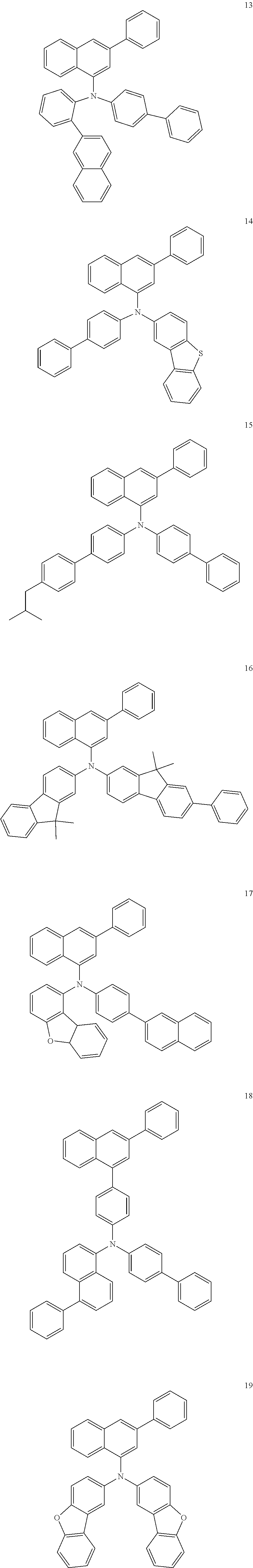

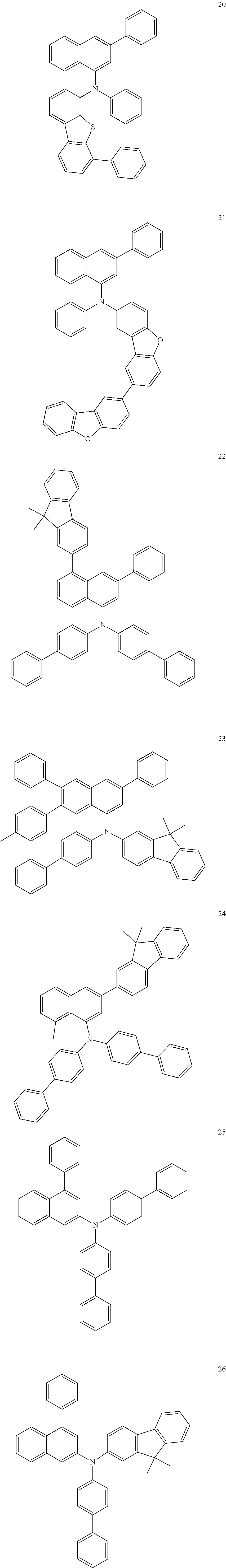

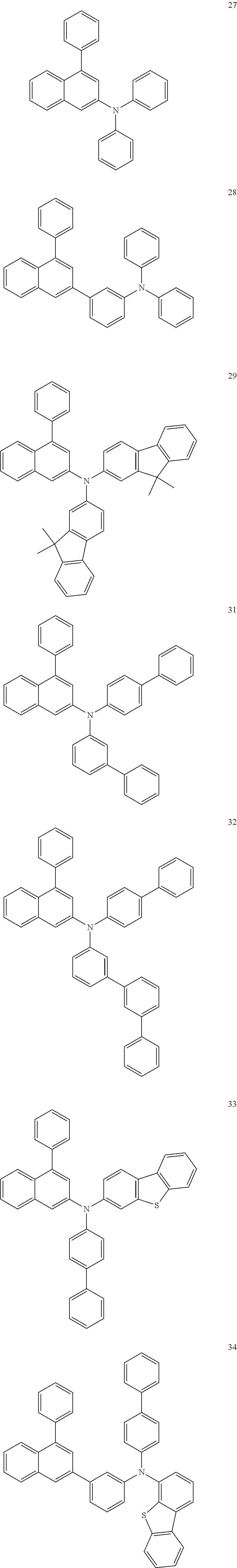

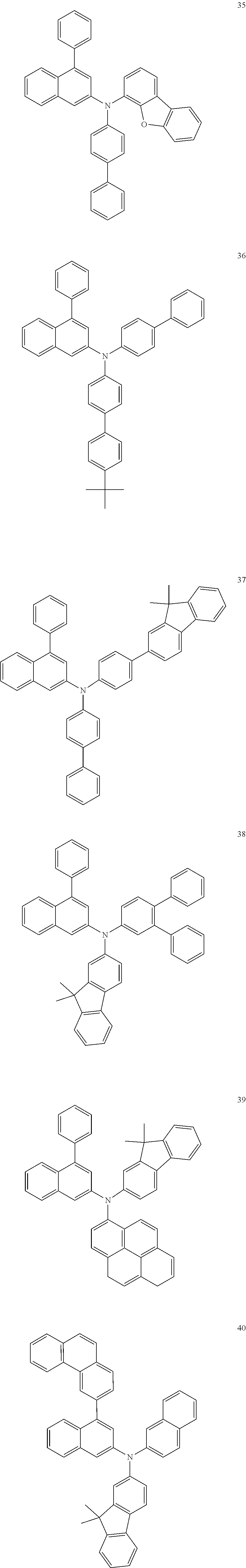 [SMILES: c1ccc(-c2ccc(N(c3cc(-c4ccccc4)c4ccccc4c3)c3cccc4c3oc3ccccc34)cc2)cc1 ; CC(C)(C)c1ccc(-c2ccc(N(c3ccc(-c4ccccc4)cc3)c3cc(-c4ccccc4)c4ccccc4c3)cc2)cc1 ; CC1(C)c2ccccc2-c2ccc(-c3ccc(N(c4ccc(-c5ccccc5)cc4)c4cc(-c5ccccc5)c5ccccc5c4)cc3)cc21 ; CC1(C)c2ccccc2-c2ccc(N(c3ccc(-c4ccccc4)c(-c4ccccc4)c3)c3cc(-c4ccccc4)c4ccccc4c3)cc21 ; CC1(C)c2ccccc2-c2ccc(N(c3cc(-c4ccccc4)c4ccccc4c3)c3ccc4c5c6c(ccc35)C=CCC6=CC4)cc21 ; CC1(C)c2ccccc2-c2ccc(N(c3ccc4ccccc4c3)c3cc(-c4ccc5ccc6ccccc6c5c4)c4ccccc4c3)cc21]

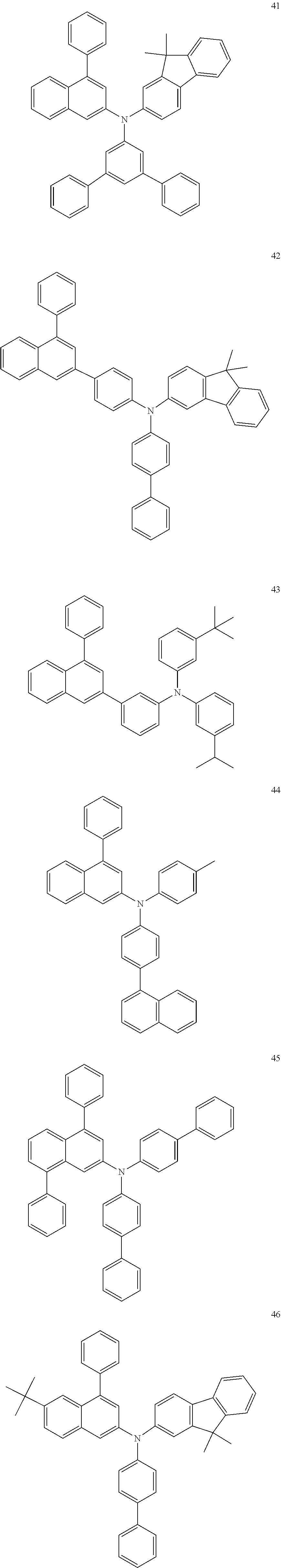

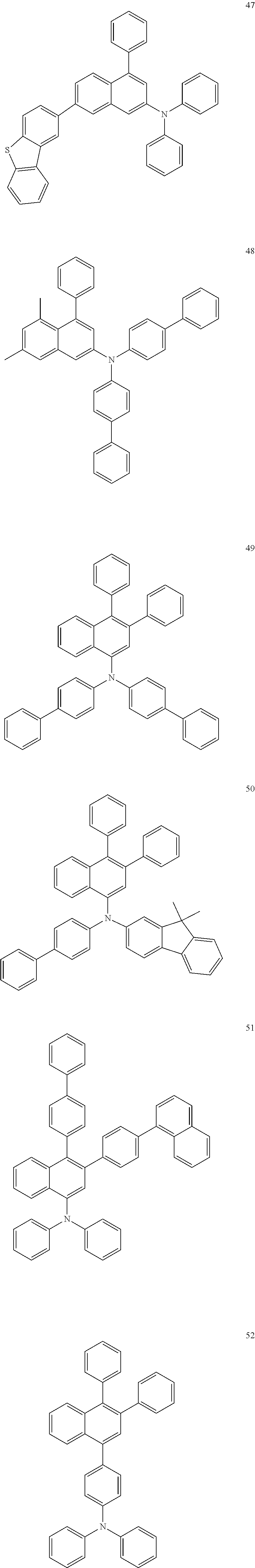 [SMILES: c1ccc(-c2cc(N(c3ccccc3)c3ccccc3)cc3cc(-c4ccc5sc6ccccc6c5c4)ccc23)cc1 ; Cc1cc(C)c2c(-c3ccccc3)cc(N(c3ccc(-c4ccccc4)cc3)c3ccc(-c4ccccc4)cc3)cc2c1 ; c1ccc(-c2ccc(N(c3ccc(-c4ccccc4)cc3)c3cc(-c4ccccc4)c(-c4ccccc4)c4ccccc34)cc2)cc1 ; CC1(C)c2ccccc2-c2ccc(N(c3ccc(-c4ccccc4)cc3)c3cc(-c4ccccc4)c(-c4ccccc4)c4ccccc34)cc21 ; c1ccc(-c2ccc(-c3c(-c4ccc(-c5cccc6ccccc56)cc4)cc(N(c4ccccc4)c4ccccc4)c4ccccc34)cc2)cc1 ; c1ccc(-c2cc(-c3ccc(N(c4ccccc4)c4ccccc4)cc3)c3ccccc3c2-c2ccccc2)cc1]

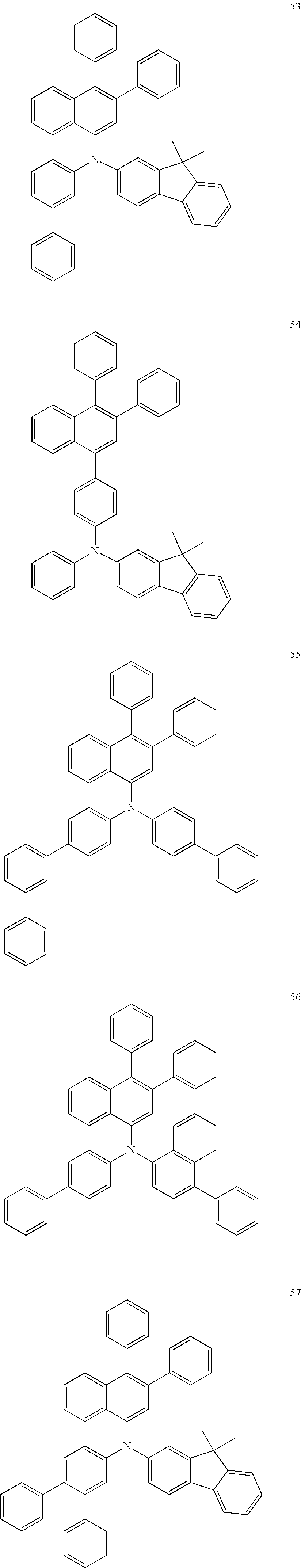 [SMILES: CC1(C)c2ccccc2-c2ccc(N(c3cccc(-c4ccccc4)c3)c3cc(-c4ccccc4)c(-c4ccccc4)c4ccccc34)cc21 ; CC1(C)c2ccccc2-c2ccc(N(c3ccccc3)c3ccc(-c4cc(-c5ccccc5)c(-c5ccccc5)c5ccccc45)cc3)cc21 ; c1ccc(-c2ccc(N(c3ccc(-c4cccc(-c5ccccc5)c4)cc3)c3cc(-c4ccccc4)c(-c4ccccc4)c4ccccc34)cc2)cc1 ; c1ccc(-c2ccc(N(c3ccc(-c4ccccc4)c4ccccc34)c3cc(-c4ccccc4)c(-c4ccccc4)c4ccccc34)cc2)cc1 ; CC1(C)c2ccccc2-c2ccc(N(c3ccc(-c4ccccc4)c(-c4ccccc4)c3)c3cc(-c4ccccc4)c(-c4ccccc4)c4ccccc34)cc21]

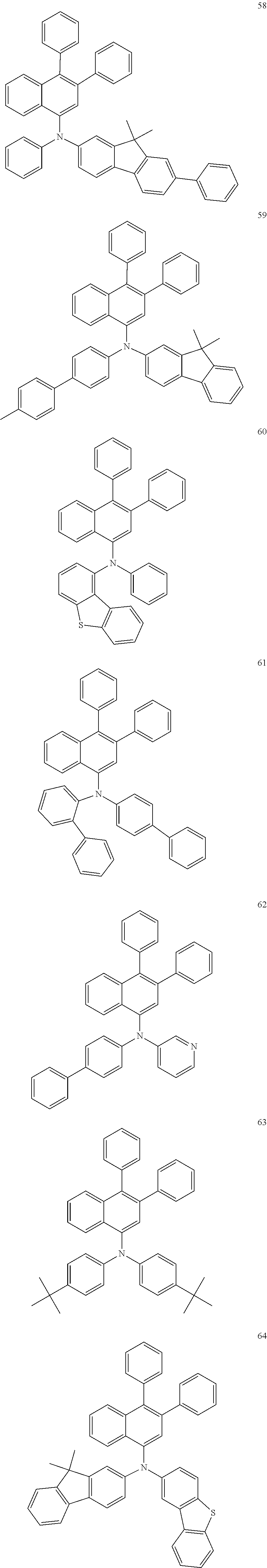

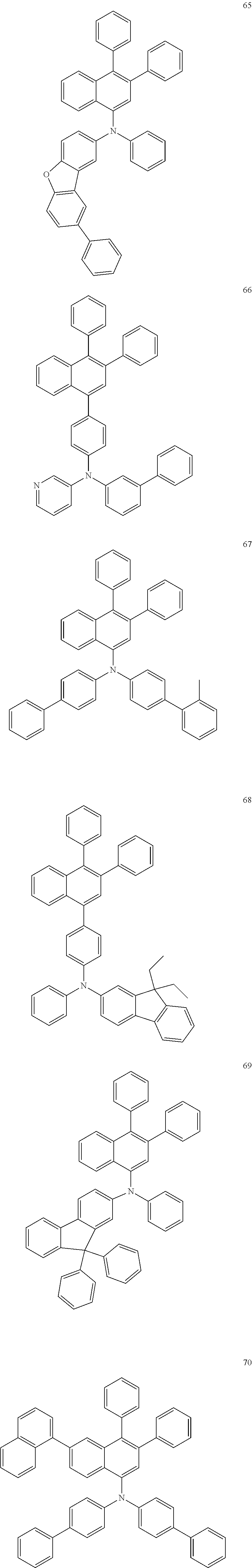

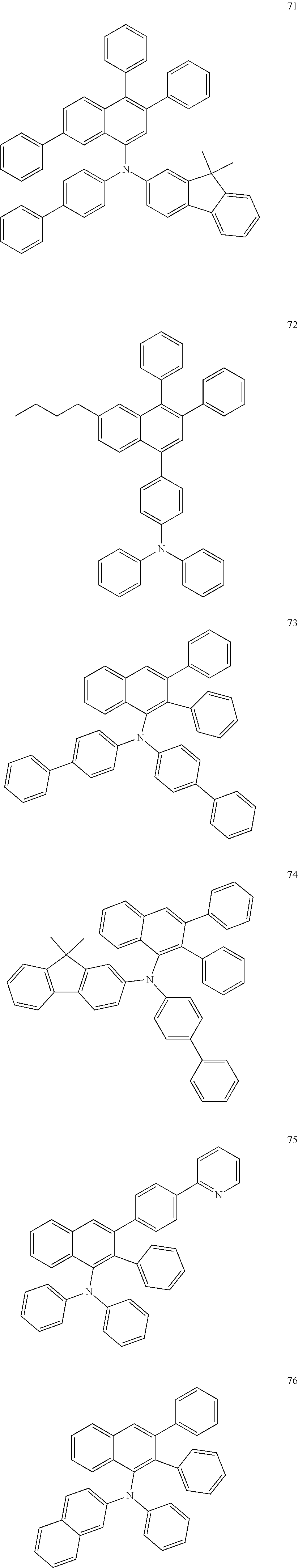

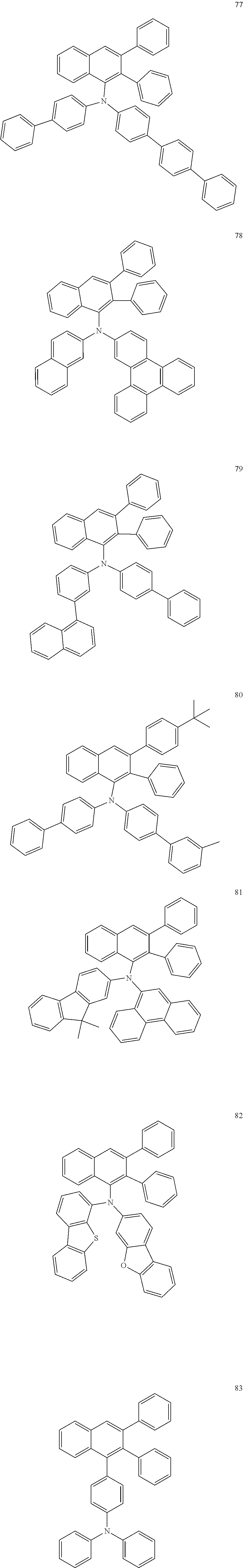

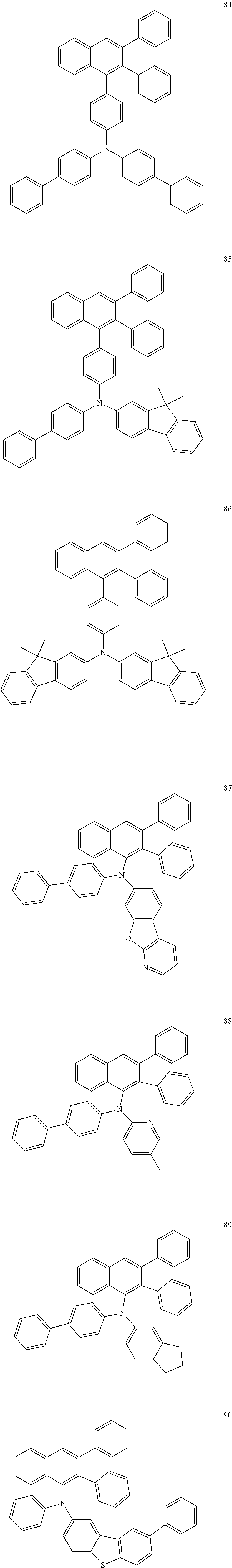

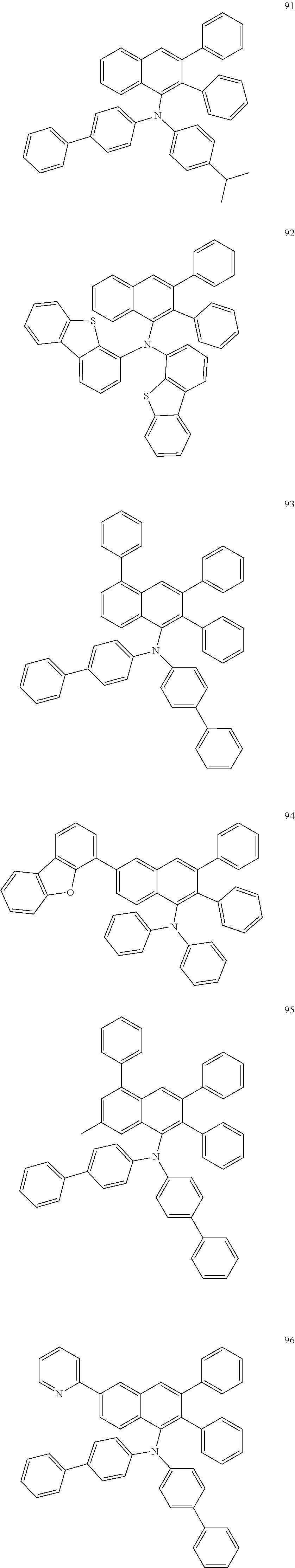

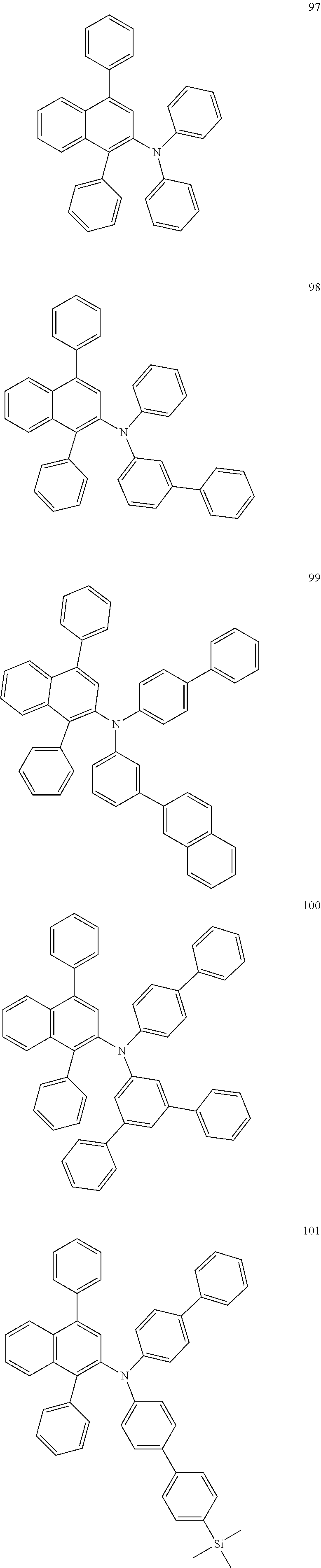

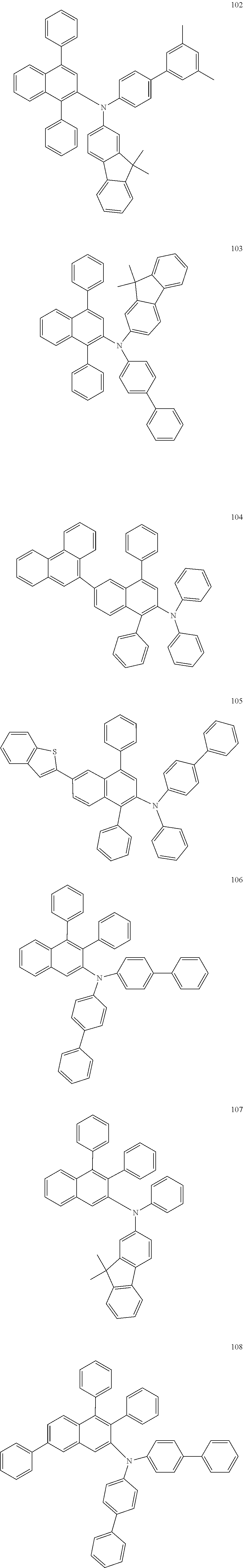

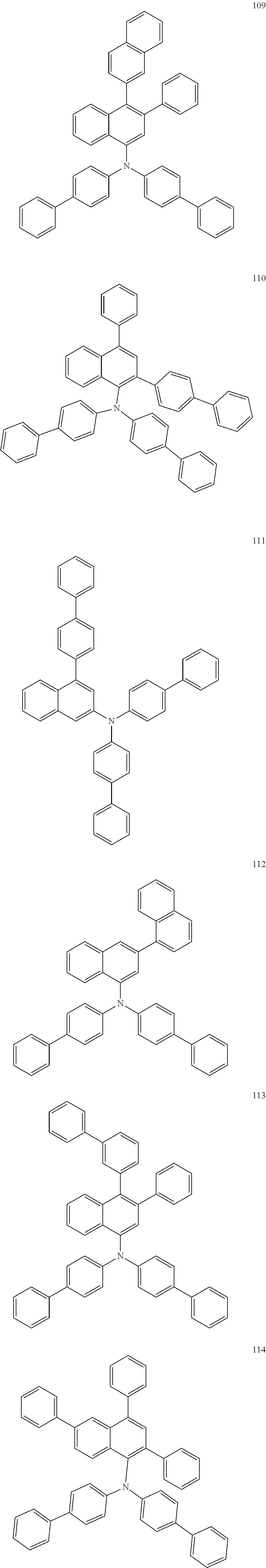

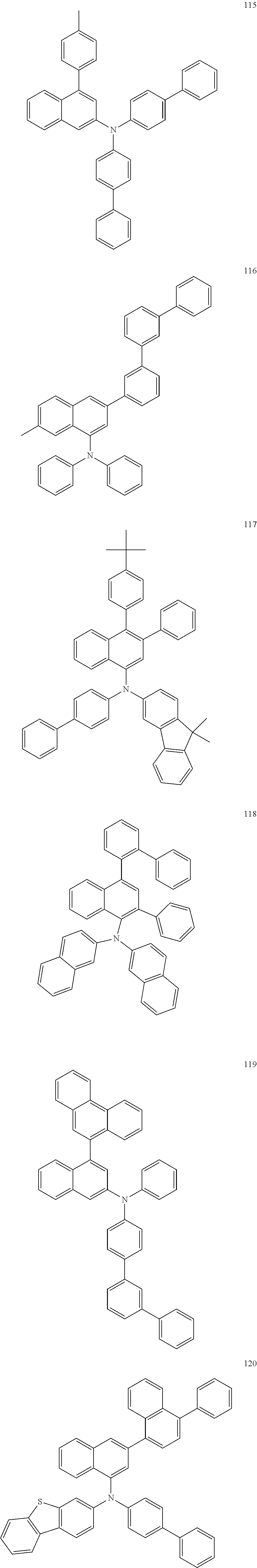 [SMILES: Cc1ccc(-c2cc(N(c3ccc(-c4ccccc4)cc3)c3ccc(-c4ccccc4)cc3)cc3ccccc23)cc1 ; Cc1ccc2cc(-c3cccc(-c4cccc(-c5ccccc5)c4)c3)cc(N(c3ccccc3)c3ccccc3)c2c1 ; CC(C)(C)c1ccc(-c2c(-c3ccccc3)cc(N(c3ccc(-c4ccccc4)cc3)c3ccc4c(c3)-c3ccccc3C4(C)C)c3ccccc23)cc1 ; c1ccc(-c2ccccc2-c2cc(-c3ccccc3)c(N(c3ccc4ccccc4c3)c3ccc4ccccc4c3)c3ccccc23)cc1 ; c1ccc(-c2cccc(-c3ccc(N(c4ccccc4)c4cc(-c5cc6ccccc6c6ccccc56)c5ccccc5c4)cc3)c2)cc1 ; c1ccc(-c2ccc(N(c3ccc4c(c3)sc3ccccc34)c3cc(-c4ccc(-c5ccccc5)c5ccccc45)cc4ccccc34)cc2)cc1]

13. An organic light emitting diode comprising a first electrode, a second electrode opposite to the first electrode, and one or more organic layers interposed between the first and second electrodes wherein one of the organic layers comprises the organic light emitting compound according to claim 12 and optionally another organic light emitting compound according to claim 12.

14. The organic light emitting diode according to claim 13, wherein the organic layers are selected from a hole injecting layer, a hole transport layer, a hole auxiliary layer, a light emitting layer, an electron transport layer, and an electron injecting layer.

15. The organic light emitting diode according to claim 13, wherein the organic light emitting compound is present in a hole auxiliary layer interposed between the first and second electrodes.
